(19) United States
(12) Patent Application Publication
(10) Pub. No.: US 2025/0362302 A1
(43) Pub. Date: Nov. 27, 2025

(54) INTRACELLULAR LIGATION OF PHOTOCATALYSTS FOR PHOTO-RESPONSIVE, PROBE-MEDIATED PROTEIN LABELING

(71) Applicant: Seed Therapeutics US, Inc., King of Prussia, PA (US)

(72) Inventors: Joel Austin, West Windsor, NJ (US); James R. Tonra, King of Prussia, PA (US); Lan Huang, King of Prussia, PA (US); Haihong Jin, King of Prussia, PA (US); Dong Liu, King of Prussia, PA (US); Xing Liu, King of Prussia, PA (US); James Finn, King of Prussia, PA (US)

(21) Appl. No.: 18/875,073

(22) PCT Filed: Jun. 13, 2023
(86) PCT No.: PCT/US2023/068382

§ 371 Date: Dec. 13, 2024

Related U.S. Application Data
(60) Provisional application No. 63/366,463, filed on Jun. 15, 2022.
(60) Provisional application No. 63/384,767, filed on Nov. 22, 2022.

(51) Int. Cl. G01N 33/68; B01J 31/00; B01J 31/18; B01J 35/39; C12N 9/14; G01N 33/573

(52) U.S. Cl. CPC G01N 33/6845; B01J 31/003; B01J 31/1815; B01J 35/39; C12N 9/14; C12Y 308/01005; G01N 33/573; B01J 2231/005; B01J 2531/827; G01N 2333/914

(57) ABSTRACT

Embodiments of the present disclosure relate to methods, compositions, and systems for proximity-based, photoactivated labeling of molecules. Molecules may be labeled via activation of a ligated photocatalyst capable of transmitting energy to a proximal biomolecular labeling agent. Depending on the activated half-life and diffusion coefficient of the labeling agent, molecules within a particular vicinity of the ligated photocatalyst may be labeled but molecules outside the vicinity will not be labeled.

### INCORPORATION BY REFERENCE TO ANY PRIORITY APPLICATIONS

This application claims the benefit of priority to U.S. Provisional Application No. 63/366,463, filed Jun. 15, 2022, and U.S. Provisional Application No. 63/384,767, filed Nov. 22, 2022, the contents of each of which are hereby incorporated by reference in their entireties.

### BACKGROUND

### Field

The present disclosure relates to compositions, systems, and methods for in vitro or in vivo proximity-based biomolecule labeling.

### Description of the Related Art

Many disease pathologies can be understood through the elucidation of localized biomolecular networks, or microenvironments. To this end, enzymatic proximity labeling platforms are broadly applied to map the extended spatial relationships in subcellular architectures. These spatial relationships between biomolecules are known to underpin fundamental biological processes. In the context of intracellular signaling, proteins may be localized within defined landscapes such as the cell membrane, which can also be considered microenvironments. The local signaling within these microenvironments may play a critical role in intercellular signaling communications. As such, the capacity to precisely map these intracellular microenvironments may yield important insights into fundamental biology, potentially having wide-reaching implications for human health and the development of novel therapeutic strategies.

Several platforms exist that facilitate the specific labeling of proteins by exploiting spatial proximity. A survey of the existing methods for targeting small molecules to protein sequences reveals that orthogonal technologies could be complementary to known methods. These technologies (including, but not limited to, APEX, Air-ID, BioID, SPPLAT, EMARS, and Turbo-ID) employ common design element wherein a tethered enzyme is genetically attached to the protein of interest. This functional enzyme catalytically generates reactive species which target specific amino-acid residues in neighboring systems through diffusion and/or physical contact. However, the reactive species which result from these technologies are known to have relatively long half-lives. As such, these reactive species may diffuse to distances comparable to or greater than that of labeling, thereby labeling proteins outside the immediate vicinity of the tethered enzyme, leading to non-specific results. Additionally, the intermediate reactive species which result from these technologies may exhibit preferential (i.e., nonagnostic) binding to particular amino acid residues. In the context of cellular signal pathway mapping, preferential binding reactivity to particular amino acid residues can lead to skewed results, as the labeling is dependent on the residues of the exposed surfaces. Even with these limitations, these technologies have revolutionized the ability to track the expression, localization and conformational changes of proteins and components in cellular signaling pathways. Given the intrinsic value of understanding biological systems at the microenvironment level, there remains a demand for cellular mapping technologies that operate with higher precision.

In particular, the aforementioned technologies have displayed a particular weakness for the identification of transiently- and/or weakly-interacting partners. Importantly, identification of transiently- and/or weakly-interacting partners is particularly problematic in the field of targeted protein degradation (TPD). For example, all known molecular glues for TPD come from an increase in the transiently- and/or weakly-interacting basal affinity between a ligase and a protein of interest. As such, the pairing of an E3 ligase to proteins targeted for degradation remains poorly understood.

Accordingly, there exists a need for a method to agnostically label intracellular proteins, peptides, chromatin, and nucleic acids at a high level of spatial resolution.

### SUMMARY

The present disclosure provides systems, compositions, and methods relating to the labeling of intracellular molecules within proximity of a protein of interest.

In one aspect, the present disclosure relates to a method of proximity-based labeling of intracellular molecules, including:
intracellularly introducing a binding agent complex to a cell, where the binding agent complex includes a protein of interest coupled to a binding agent, where the binding agent is capable of binding to a catalyst complex;
introducing the catalyst complex to the cell, thereby forming a biomolecular antenna including the catalyst complex and the binding agent complex;
introducing a labeling agent including a label moiety and a reactive moiety, to the cell, wherein the reactive moiety is configured to be activated to a reactive state by the catalyst complex; and
activating the catalyst complex, thereby activating the labeling agent by transfer of energy from the catalyst complex to the reactive moiety and causing the labeling agent to bind to a biomolecule within the cell.

In some embodiments, introducing the binding agent complex to the cell includes introducing a nucleotide construct to the cell, wherein the nucleotide construct encodes for the binding agent complex, and wherein the nucleotide construct can be expressed by the cell. In further embodiments, introducing a nucleotide construct to a cell includes transfecting the cell.

In some embodiments, the biomolecule is an intracellular protein, a peptide, a chromatin, or a nucleic acid.

In some embodiments, the method further includes imaging a signal from the label moiety.

In some embodiments, the catalyst complex includes a photocatalyst. In further embodiments, the photocatalyst includes a transition metal complex. In yet further embodiments, activating the transition metal complex includes photoactivating the transition metal complex. In yet further embodiments, photoactivating includes shining light on the transition metal complex, where the light has a wavelength from about 380 nm to about 700 nm. In some embodiments, activating the catalyst complex causes a Dexter energy transfer from the activated transition metal complex to the reactive moiety to form a reactive intermediate.

In some embodiments, the biomolecule is within a 10 nm radius of the biomolecular antenna when the labeling agent binds to it.

In another aspect, the present disclosure provides a system for proximity-based labeling of intracellular molecules, including:
a cell;
a biomolecular antenna comprising:
a photocatalyst complex, including a photocatalyst and a ligand moiety, and
a binding agent complex, including a protein of interest coupled to a binding agent, where the binding agent is capable of binding the ligand moiety of the photocatalyst complex; and
a labeling agent including a label moiety and a reactive moiety, where the reactive moiety is configured to be activated to a reactive state by the photocatalyst complex;
where the biomolecular antenna and the labeling agent are each located within the cell.

In some embodiments, the binding agent includes a haloalkane dehalogenase. In further embodiments, the ligand moiety includes an alkyl chloride. In yet further embodiments, the alkyl chloride includes 6 or more carbons.

In some embodiments, the binding agent is coupled to the protein of interest at the N-terminus or C-terminus of the protein of interest.

In some embodiments, the ligand moiety includes 4,4′-di-tert-butyl-2,2′-dipyridyl, 2,2′-bipyridine, diphenhydramine-2,2′-bipyridine, 4-4′-dimethoxy-2-2′-bipyridine, dinapthalene-pyrene, phenanthroline, or diphenyl-phenanthroline.

In some embodiments, the photocatalyst includes a transition metal. In further embodiments, the transition metal has a triplet energy state greater than 60 kcal/mol. In further embodiments, the transition metal is a platinum group metal. In yet further embodiments, the transition metal is iridium or ruthenium. In further embodiments, the transition metal is hexacoordinate. In further embodiments, the transition metal absorbs light having wavelength from about 380 nm to about 700 nm. In further embodiments, the transition metal has a visible light extinction coefficient greater than 1000 M⁻¹cm⁻¹.

In some embodiments, the photocatalyst is an organocatalyst not including a transition metal. In further embodiments, the organocatalyst includes a thioxanthone group, phenothiazine group, flavin group, phenoxazine group, pthalazine group, quinoxaline group, quinazoline group, benzophenothiazine group, coumarin group, acetophenone group, or benzophenone group.

In some embodiments, the labeling agent is cell-permeable.

In some embodiments, the photocatalyst is capable of activating the labeling agent to form a reactive intermediate. In further embodiments, the photocatalyst is capable of activating the labeling agent to form the reactive intermediate via Dexter energy transfer. In further embodiments, the reactive intermediate has a diffusion radius of less than 10 nm prior to quenching. In further embodiments, the reactive intermediate has a half-life (t_{1/2}) less than 2 ns.

In some embodiments, the binding agent is a protein, an E3 ligase, a polysaccharide, or a nucleic acid.

In some embodiments, the protein of interest is a K-Ras, a cMyc, a Src, a WRN, a Slug, a PARP1, an Aβ, a Tau, an influenza hemagglutinin, or a viral nucleoprotein.

In another aspect, the present disclosure provides a biomolecular assembly, including a dehalogenase coupled to a protein of interest and a photocatalyst.

In some embodiments, the photocatalyst is configured to activate a labeling agent comprising a label moiety and a reactive moiety. In further embodiments, the photocatalyst is configured to activate the labeling agent after absorbing light having a wavelength from about 380 nm to about 700 nm.

In some embodiments, the photocatalyst includes a transition metal. In further embodiments, the transition metal is a platinum group metal. In yet further embodiments, the transition metal is iridium, tin, or ruthenium. In further embodiments, the transition metal is hexacoordinate. In further embodiments, the transition metal has a visible light extinction coefficient greater than 1000 M⁻¹ cm⁻¹.

In some embodiments, the photocatalyst is an organocatalyst not including a transition metal. In further embodiments, the organocatalyst includes a thioxanthone group, phenothiazine group, flavin group, phenoxazine group, pthalazine group, quinoxaline group, quinazoline group, benzophenothiazine group, coumarin group, acetophenone group, or benzophenone group.

In some embodiments, the biomolecular assembly further includes a photocatalyst complex, where the photocatalyst complex includes the photocatalyst and a ligand moiety. In further embodiments, the ligand moiety includes an alkyl chloride. In yet further embodiments, the alkyl chloride includes 6 or more carbons.

In another aspect, the present disclosure provides a method of detecting a protein-protein interaction, including:
intracellularly expressing a first protein in a cell, the first protein coupled to a binding agent capable of binding to a catalyst complex, wherein the catalyst complex is a photocatalyst;
introducing the photocatalyst to the cell, thereby causing it to bind to the first protein;
introducing a labeling agent comprising a label moiety and a reactive moiety to the cell;
activating the photocatalyst, thereby activating the labeling agent by transfer of energy from the photocatalyst to the reactive moiety and causing the labeling agent to bind to a second protein within the cell; and
detecting the second protein by detecting the label moiety;
wherein the photocatalyst has the structure of Formula (I):

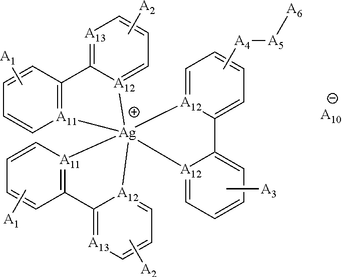

where:
A1 is present 0-4 times on the ring to which it is attached and each A1 is independently selected from CH₃, CF₃, F, Cl, N(CH₃)₂, and OR₁;
each R₁ is independently selected from H, a linear or branched alkyl group having 1-12 carbons, CHF₂, and CF₃;
A2 is present 0-4 times on the ring to which it is attached and each A2 is independently selected from CH₃, CF₃, F, Cl, N(CH₃)₂, and OR₁;
A3 is present 0-4 times on the ring to which it is attached and each A3 is independently selected from CH₃, CF₃, F, Cl, N(CH₃)₂, and OR₁;
A4 is selected from null, linear or branched alkyl group having 1-12 carbons, C₆₋₁₀ aryl, C₃₋₈ cycloalkyl, 4-10 membered heterocyclyl, and 5-10 membered heteroaryl, wherein said alkyl, aryl, cycloalkyl, heterocyclyl, and heteroaryl is optionally substituted with one or more C₁₋₆ alkyl, C₁₋₆ haloalkyl, halo, hydroxy, C₁₋₆ alkoxy, or amino;
A5 is selected from CONH, NHCO, SONH, SO₂NH, NHSO, NHSO₂, NH, OCONH, NHCOO, C₁₋₆ alkoxy, and O

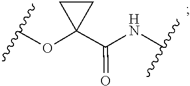 [SMILES: *NC(=O)C1(O*)CC1]

A6 is independently (PEG)a₁(CH₂)a₂Cl or (PEG)a₁(CH₂)a₂(5-6 membered heterocyclyl)(PEG)a₁(CH₂)a₂Cl, wherein each a₁ is independently an integer from 0-10 and each a₂ is independently an integer from 6-10;
A9 is iridium;
A10 is an anion selected from tetraalkylborate, tetrafluoroborate, tetraphenylborate, chloride, cyanide, hexafluorophosphate (PF₆), bis(triphenylphosphine)iminium chloride, tetraphenylphosphonium chloride, and tetrabutylammonium;
each A11 and A12 are independently a C or N coordinated to A9; and
each A13 is independently CH or N.

In some embodiments, A3 may be present 0-4 times on the ring to which it is attached and each A3 may be independently selected from CH₃, CF₃, F, Cl, and OR₁. In some embodiments, A5 may be selected from CONH, NHCO, SONH, SO₂NH, NHSO, NHSO₂, NH, OCONH, and NHCOO. In some embodiments, A6 may be (PEG)a₁(CH₂)a₂Cl, a₁ may be an integer from 0-10 and a₂ may be an integer from 6-10. In some embodiments, A13 may be CH. In some embodiments, the 5-6 membered heterocyclyl of A6 may be piperazine or pyrrolidine. In some embodiments, the photocatalyst has the structure:

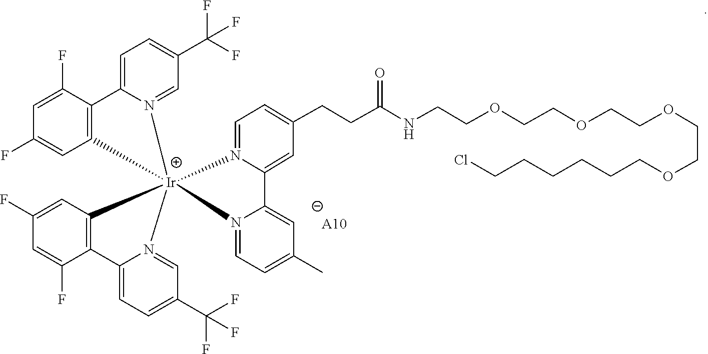

In some embodiments, the photocatalyst has the structure:

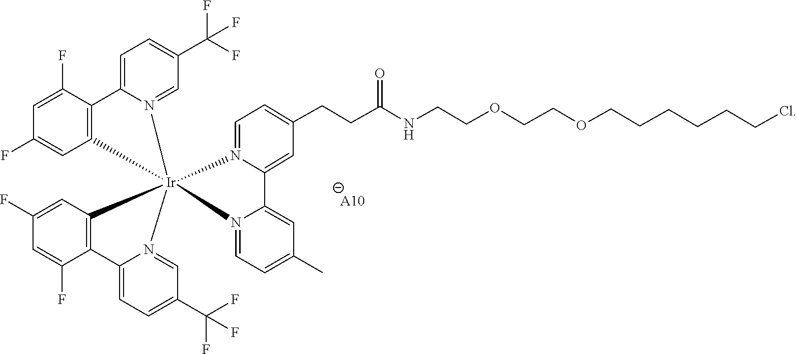

In some embodiments, the photocatalyst has the structure:

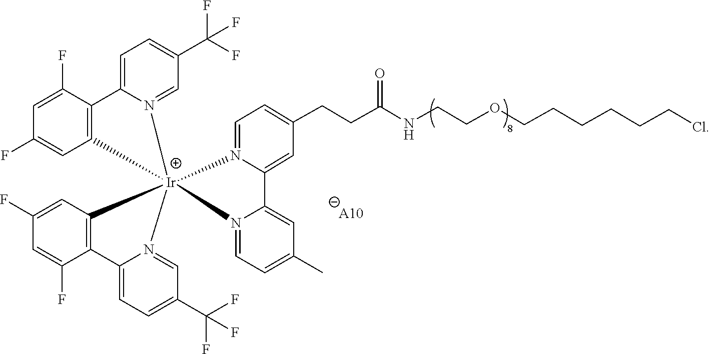

In some embodiments, the photocatalyst has the structure:

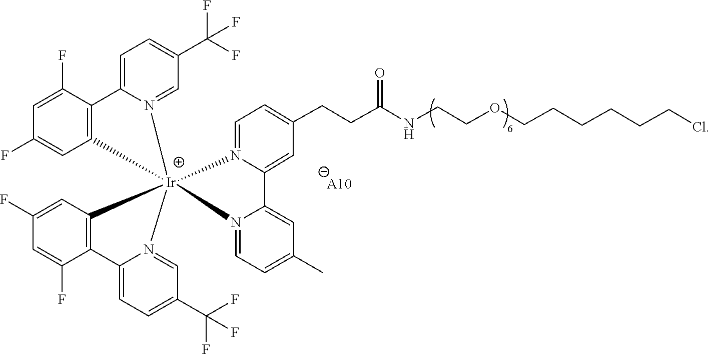

In some embodiments, the photocatalyst has the structure:

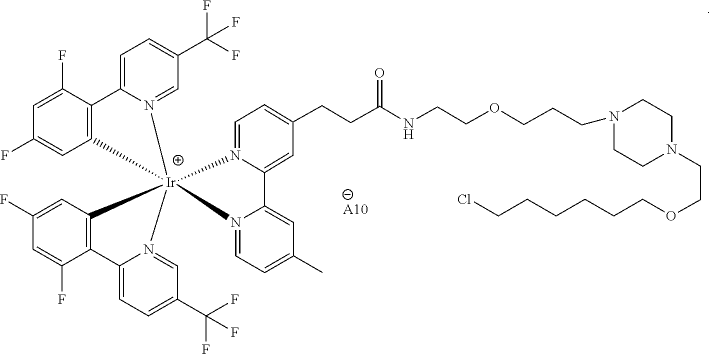

In some embodiments, the photocatalyst has the structure:

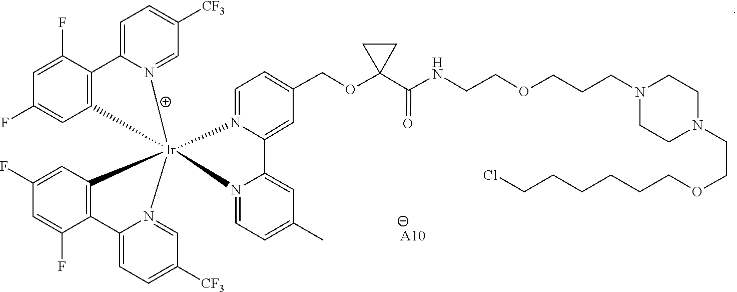

In some embodiments, the photocatalyst has the structure:

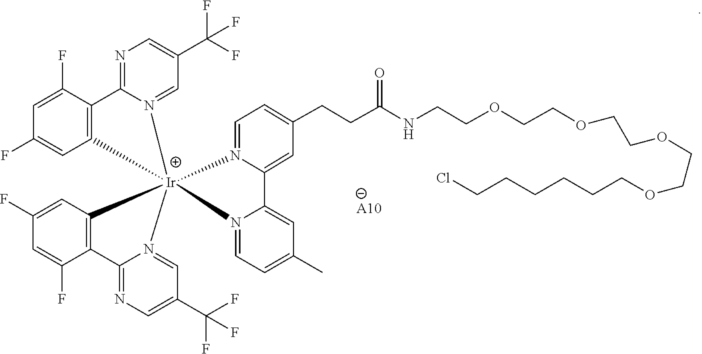

In some embodiments, the photocatalyst has the structure:

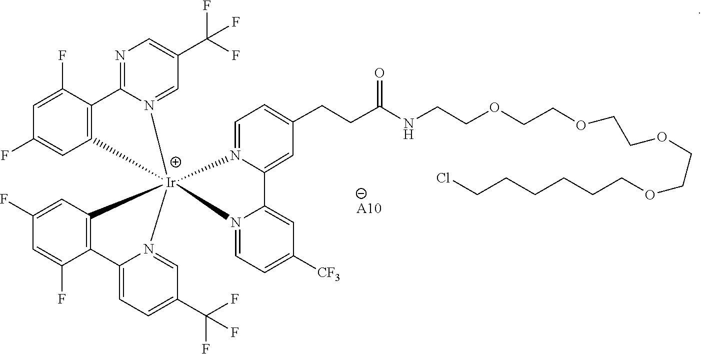

In some embodiments, the photocatalyst has the structure:

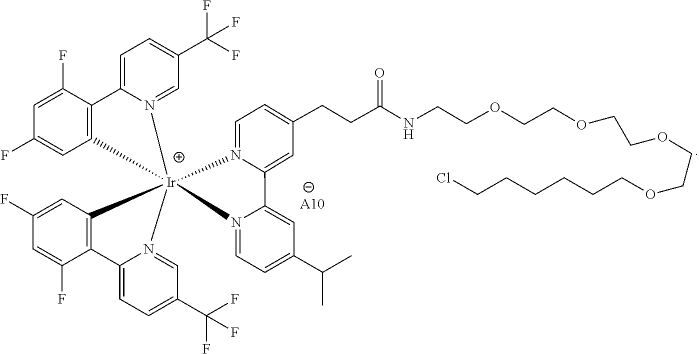

In some embodiments, the photocatalyst has the structure:

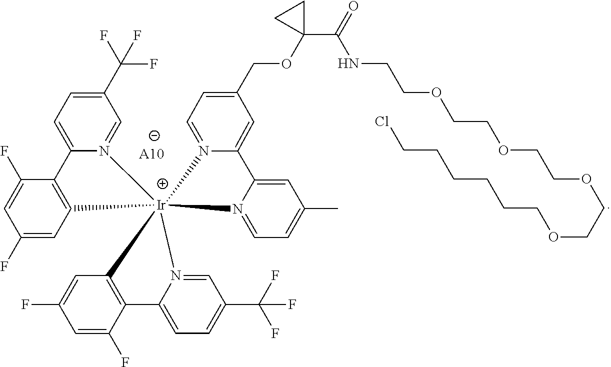

In another aspect, the present disclosure provides a method of detecting a protein-protein interaction, including:
intracellularly expressing a first protein in a cell, the first protein coupled to a binding agent capable of binding to a catalyst complex;
introducing the catalyst complex to the cell, thereby causing it to bind to the first protein;
introducing a labeling agent comprising a label moiety and a reactive moiety to the cell;
activating the catalyst complex, thereby activating the labeling agent by transfer of energy from the catalyst complex to the reactive moiety and causing the labeling agent to bind to a second protein within the cell; and
detecting the second protein by detecting the label moiety;
wherein the labeling agent has the structure of Formula (III-a):

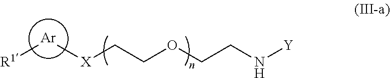

wherein:
R¹′ is selected from an azide, a methyl diazirine, a trifluoromethyl diazirine, and a phenyl diazirine;
Ring Ar is selected from phenyl, pyridyl, pyrimidyl, pyrazinyl, pyridizynyl, naphthyl, and quinolinyl, optionally substituted with one or more —OH, —OMe, —OEt, —OCF₃, —OCF₂H, —NHMe, —NMe₂, —F, —Cl, —Br, -Me, or -Et;
X is selected from O, NH, NR², CH₂NHCO, CONH, CONR², SO₂NH, and SO₂NR²;
R² is selected from H, OMe, Me, and Et;
n is 0, 1, 2, 3, 4, 5, or 6; and
Y is a biotin-linked amide or an amide-linked fluorescent dye.

In some embodiments, the labeling agent has the structure:

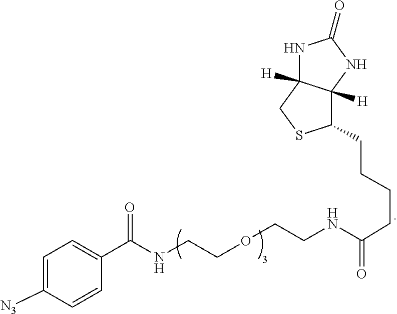

In some embodiments of the methods disclosed herein, the first protein may be a ubiquitin ligase.

In another aspect, the present disclosure provides a method of detecting a protein-protein interaction, including:
intracellularly expressing a first protein in a cell, the first protein coupled to a binding agent capable of binding to a catalyst complex, wherein the first protein is a ubiquitin ligase;
introducing the catalyst complex to the cell, thereby causing it to bind to the first protein;
introducing a labeling agent comprising a label moiety and a reactive moiety to the cell;
activating the catalyst complex, thereby activating the labeling agent by transfer of energy from the catalyst complex to the reactive moiety and causing the labeling agent to bind to a second protein within the cell; and
detecting the second protein by detecting the label moiety.

In some embodiments of the methods disclosed herein, the catalyst complex is a photocatalyst. In some embodiments, the photocatalyst has the structure of Formula (I):

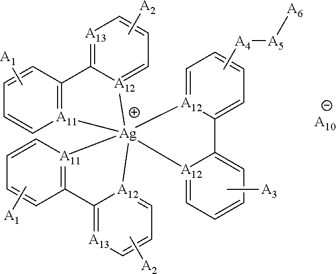

where:
A1 is present 0-4 times on the ring to which it is attached and each A1 is independently selected from CH₃, CF₃, F, Cl, N(CH₃)₂, and OR₁;
each R₁ is independently selected from H, a linear or branched alkyl group having 1-12 carbons, CHF₂, and CF₃;
A2 is present 0-4 times on the ring to which it is attached and each A2 is independently selected from CH₃, CF₃, F, Cl, N(CH₃)₂, and OR₁;
A3 is present 0-4 times on the ring to which it is attached and each A3 is independently selected from CH₃, CF₃, F, Cl, N(CH₃)₂, and OR₁;
A4 is selected from null, linear or branched alkyl group having 1-12 carbons, C₆₋₁₀ aryl, C₃₋₈ cycloalkyl, 4-10 membered heterocyclyl, and 5-10 membered heteroaryl, wherein said alkyl, aryl, cycloalkyl, heterocyclyl, and heteroaryl is optionally substituted with one or more C₁₋₆ alkyl, C₁₋₆ haloalkyl, halo, hydroxy, C₁₋₆ alkoxy, or amino;
A5 is selected from CONH, NHCO, SONH, SO₂NH, NHSO, NHSO₂, NH, OCONH, NHCOO, C₁₋₆ alkoxy, and spiral C₁₋₆ alkoxy;
A6 is independently (PEG)a₁(CH₂)a₂Cl or (PEG)a₁(CH₂)a₂(5-6 membered heterocyclyl)(PEG)a₁(CH₂)a₂Cl, wherein each a₁ is independently an integer from 0-10 and each a₂ is independently an integer from 6-10;
A9 is a D9 metal selected from copper, vanadium, chromium, scandium, titanium, manganese, iron, cobalt, nickel, copper, zinc, yttrium, zirconium, niobium, molybdenum, technetium, ruthenium, rhodium, palladium, silver, cadmium lanthanum, hafnium, tantalum, tungsten, rhenium, osmium, iridium, platinum, gold, and mercury;
A10 is an anion selected from tetraalkylborate, tetrafluoroborate, tetraphenylborate, chloride, cyanide, hexafluorophosphate (PF₆), bis(triphenylphosphine)iminium chloride, tetraphenylphosphonium chloride, and tetrabutylammonium;
A11 and A12 are independently a C or N coordinated to A9; and
each A13 is independently CH or N.

In some embodiments, A3 may be present 0-4 times on the ring to which it is attached and each A3 may be independently selected from CH₃, CF₃, F, Cl, and OR₁. In some embodiments, A5 may be selected from CONH, NHCO, SONH, SO₂NH, NHSO, NHSO₂, NH, OCONH, and NHCOO. In some embodiments, A6 may be (PEG)a₁(CH₂)a₂Cl, a₁ may be an integer from 0-10 and a₂ may be an integer from 6-10. In some embodiments, A13 may be CH. In some embodiments, the 5-6 membered heterocyclyl of A6 may be piperazine or pyrrolidine. In some embodiments, the photocatalyst has the structure:

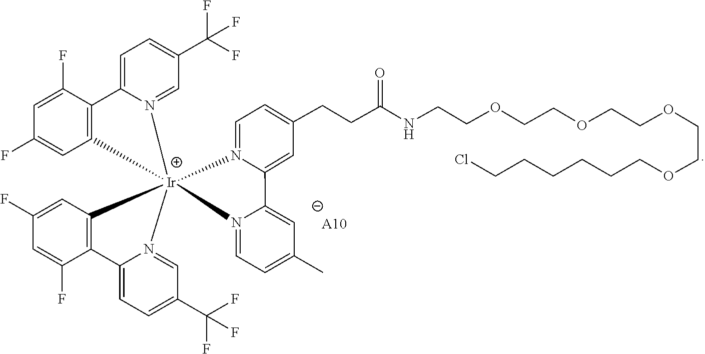

In some embodiments, the photocatalyst has the structure:

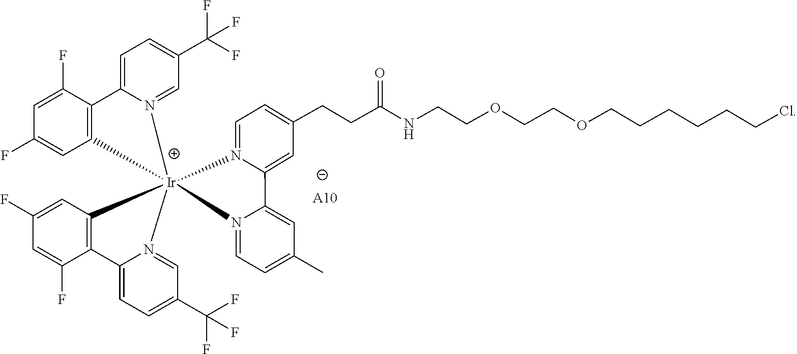

In some embodiments, the photocatalyst has the structure:

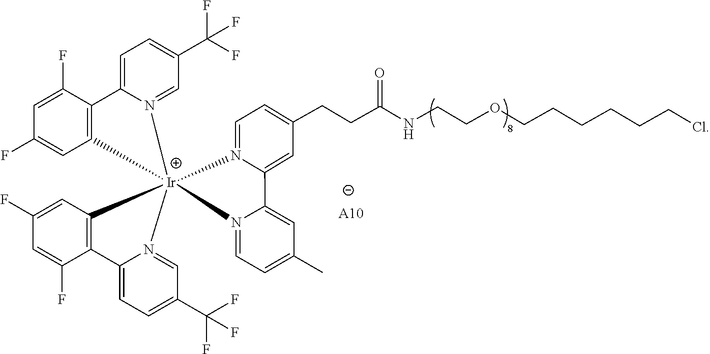

In some embodiments, the photocatalyst has the structure:

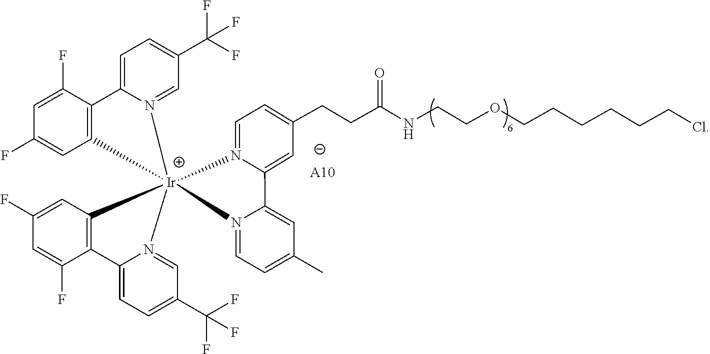

In some embodiments, the photocatalyst has the structure:

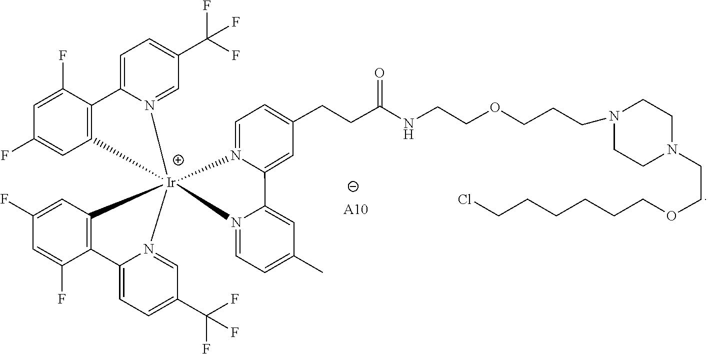

In some embodiments, the photocatalyst has the structure:

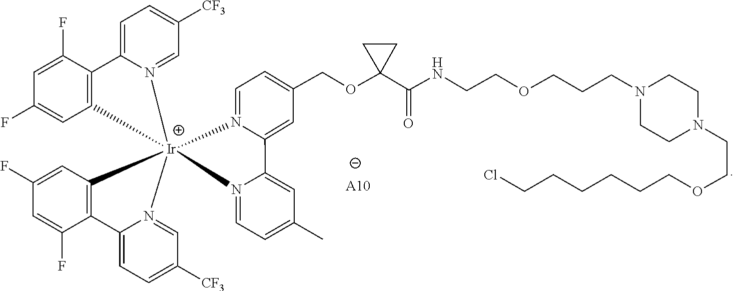

In some embodiments, the photocatalyst has the structure:

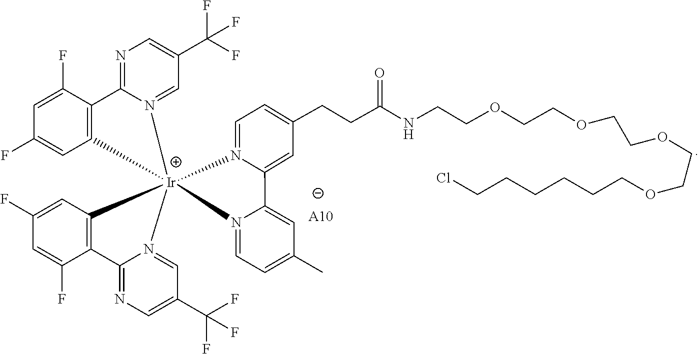

In some embodiments, the photocatalyst has the structure:

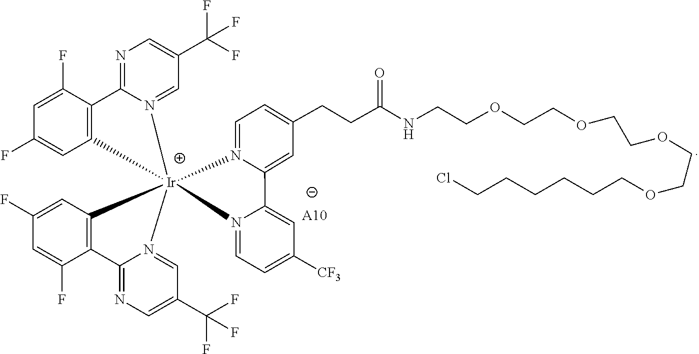

In some embodiments, the photocatalyst has the structure:

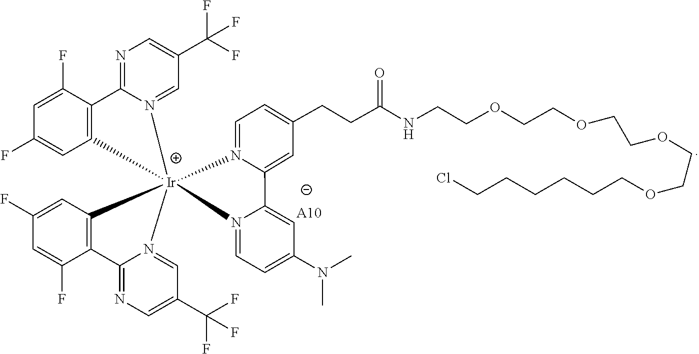

In some embodiments, the photocatalyst has the structure:

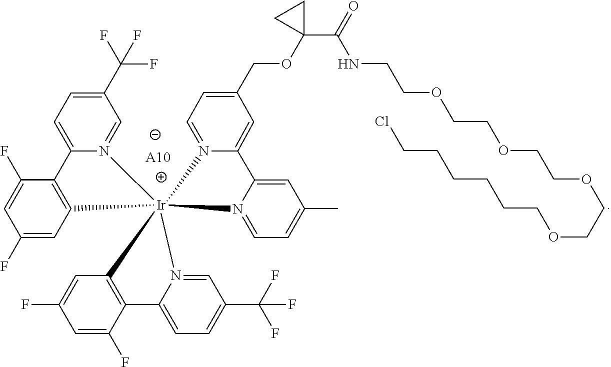

In some embodiments of methods disclosed herein, the labeling agent has the structure of Formula (III-a):

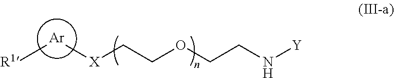

where:
R¹′ is selected from an azide, a methyl diazirine, a trifluoromethyl diazirine, and a phenyl diazirine;
Ring Ar is selected from phenyl, pyridyl, pyrimidyl, pyrazinyl, pyridizynyl, naphthyl, and quinolinyl, optionally substituted with one or more —OH, —OMe, —OEt, —OCF₃, —OCF₂H, —NHMe, —NMe₂, —F, —Cl, —Br, -Me, or -Et;
X is selected from 0, NH, NR², CH₂NHCO, CONH, CONR², SO₂NH, and SO₂NR²;
R² is selected from H, OMe, Me, and Et;
n is 0, 1, 2, 3, 4, 5, or 6; and
Y is a biotin-linked amide or an amide-linked fluorescent dye.

In some embodiments, the labeling agent has the structure:

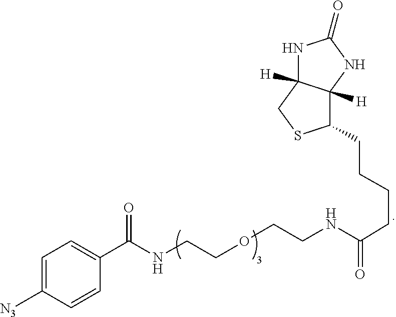

In some embodiments of methods disclosed herein, the method is conducted in the absence of an exogenous compound that promotes interaction between the first and second proteins.

In some embodiments of methods disclosed herein, the method is conducted in the presence of a test compound, wherein detecting the second protein or a level of the second protein indicates that the test compound promotes interaction between the first and second proteins.

In some embodiments of methods disclosed herein, during the activating of the catalyst complex, the cell is a live cell. In some embodiments of methods disclosed herein, the binding agent comprises a haloalkane dehalogenase. In some embodiments of methods disclosed herein, activating the catalyst complex comprises shining light on the cell, wherein the light has a wavelength from about 380 nm to about 700 nm. In some embodiments of methods disclosed herein, activating the catalyst complex causes a Dexter energy transfer from the activated catalyst complex to the reactive moiety to form a reactive intermediate.

In another aspect, the present disclosure provides a cell, including:
a first protein coupled to a haloalkane dehalogenase;
a photocatalyst having the structure of Formula (I):

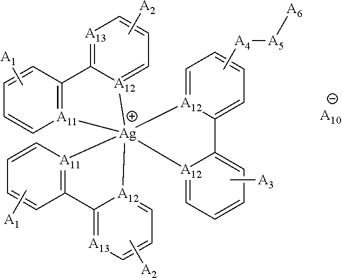

wherein:
A1 is present 0-4 times on the ring to which it is attached and each A1 is independently selected from CH₃, CF₃, F, Cl, N(CH₃)₂, and OR₁;
each R₁ is independently selected from H, a linear or branched alkyl group having 1-12 carbons, CHF₂, and CF₃;
A2 is present 0-4 times on the ring to which it is attached and each A2 is independently selected from CH₃, CF₃, F, Cl, N(CH₃)₂, and OR₁;
A3 is present 0-4 times on the ring to which it is attached and each A3 is independently selected from CH₃, CF₃, F, Cl, N(CH₃)₂, and OR₁;
A4 is selected from null, linear or branched alkyl group having 1-12 carbons, C₆₋₁₀ aryl, C₃₋₈ cycloalkyl, 4-10 membered heterocyclyl, and 5-10 membered heteroaryl, wherein said alkyl, aryl, cycloalkyl, heterocyclyl, and heteroaryl is optionally substituted with one or more C₁₋₆ alkyl, C₁₋₆ haloalkyl, halo, hydroxy, C₁₋₆ alkoxy, or amino;
A5 is selected from CONH, NHCO, SONH, SO₂NH NHSO NHSO₂, NH, OCONH, NHCOO, C₁₋₆ alkoxy, C₁₋₆ alkoxy, and

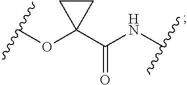 [SMILES: *NC(=O)C1(O*)CC1]

A6 is independently (PEG)a₁(CH₂)a₂C₁ or (PEG)a₁(CH₂)a₂(5-6 membered heterocyclyl)(PEG)a₁(CH₂)a₂Cl, wherein each a₁ is independently an integer from 0-10 and each a₂ is independently an integer from 6-10;
A9 is a D9 metal selected from copper, vanadium, chromium, scandium, titanium, manganese, iron, cobalt, nickel, copper, zinc, yttrium, zirconium, niobium, molybdenum, technetium, ruthenium, rhodium, palladium, silver, cadmium lanthanum, hafnium, tantalum, tungsten, rhenium, osmium, iridium, platinum, gold, and mercury;
A11 and A12 are independently a C or N coordinated to A9;
each A13 is independently CH or N; and
a labeling agent having the structure of Formula (III-a):

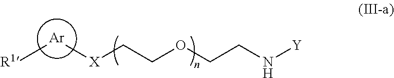

wherein:
R¹′ is selected from an azide, a methyl diazirine, a trifluoromethyl diazirine, and a phenyl diazirine;
Ring Ar is selected from phenyl, pyridyl, pyrimidyl, pyrazinyl, pyridizynyl, naphthyl, and quinolinyl, optionally substituted with one or more —OH, —OMe, —OEt, —OCF₃, —OCF₂H, —NHMe, —NMe₂, —F, —Cl, —Br, -Me, or -Et;
X is selected from 0, NH, NR², CH₂NHCO, CONH, CONR², SO₂NH, and SO₂NR²;
R² is selected from H, OMe, Me, and Et;
n is 0, 1, 2, 3, 4, 5, or 6; and
Y is a biotin-linked amide or an amide-linked fluorescent dye.

In some embodiments, A3 may be present 0-4 times on the ring to which it is attached and each A3 may be independently selected from CH₃, CF₃, F, Cl, and OR₁. In some embodiments, A5 may be selected from CONH, NHCO, SONH, SO₂NH, NHSO, NHSO₂, NH, OCONH, and NHCOO. In some embodiments, A6 may be (PEG)a₁(CH₂)a₂Cl, a₁ may be an integer from 0-10 and a₂ may be an integer from 6-10. In some embodiments, A13 may be CH. In some embodiments, the 5-6 membered heterocyclyl of A6 may be piperazine or pyrrolidine. In some embodiments, the photocatalyst has the structure:

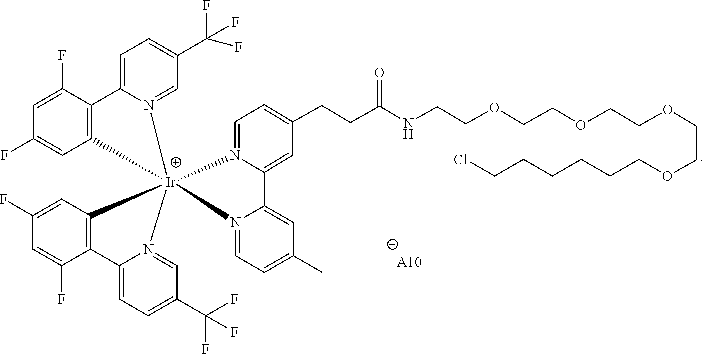

In some embodiments, the photocatalyst has the structure:

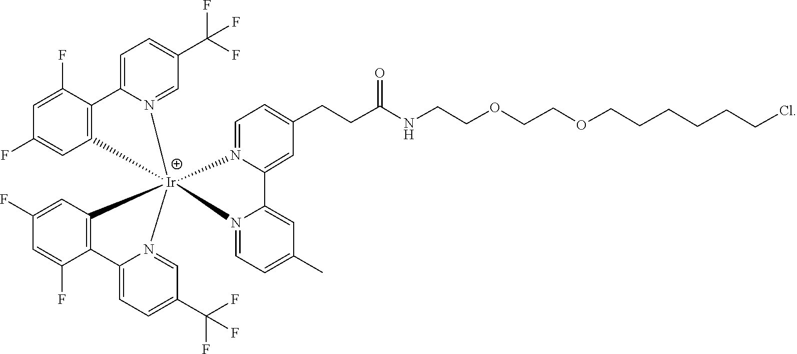

In some embodiments, the photocatalyst has the structure:

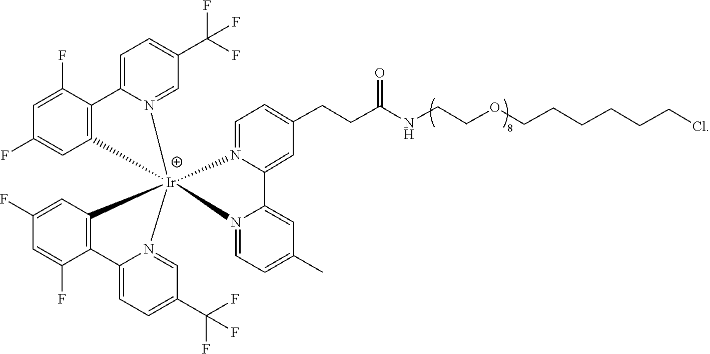

In some embodiments, the photocatalyst has the structure:

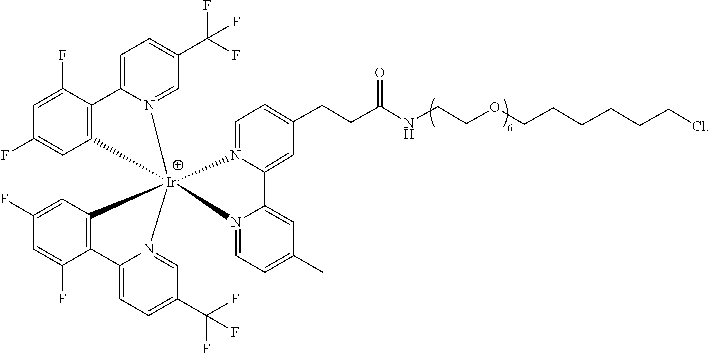

In some embodiments, the photocatalyst has the structure:

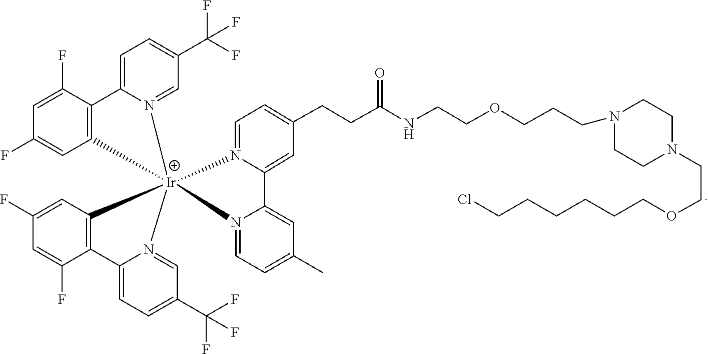

In some embodiments, the photocatalyst has the structure:

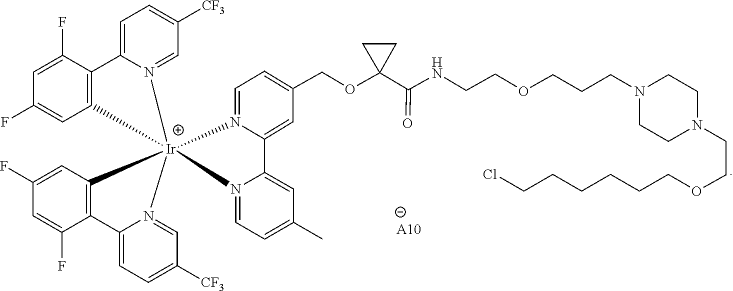

In some embodiments, the photocatalyst has the structure:

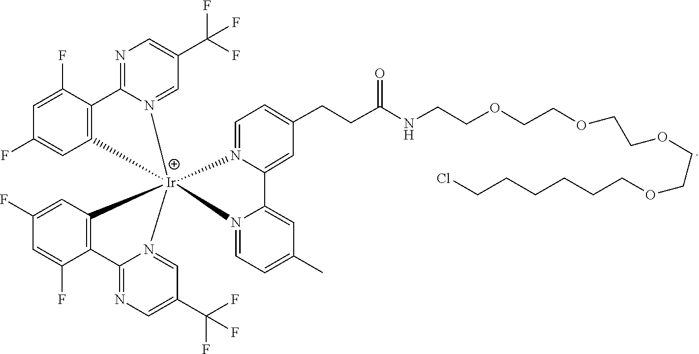

In some embodiments, the photocatalyst has the structure:

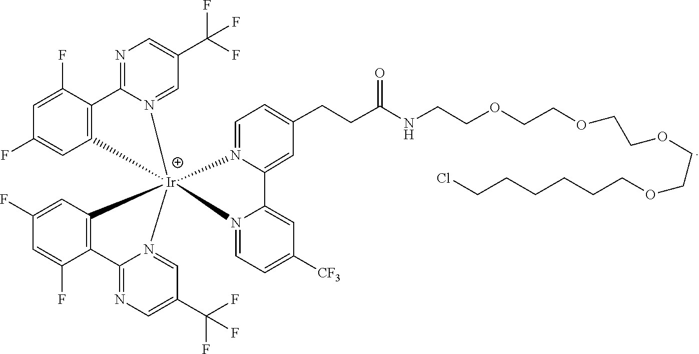

In some embodiments, the photocatalyst has the structure:

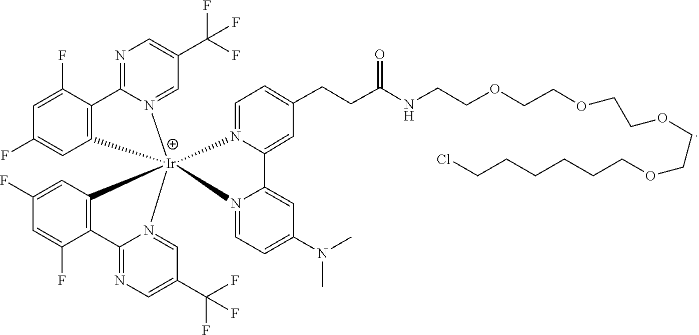

In some embodiments, the photocatalyst has the structure:

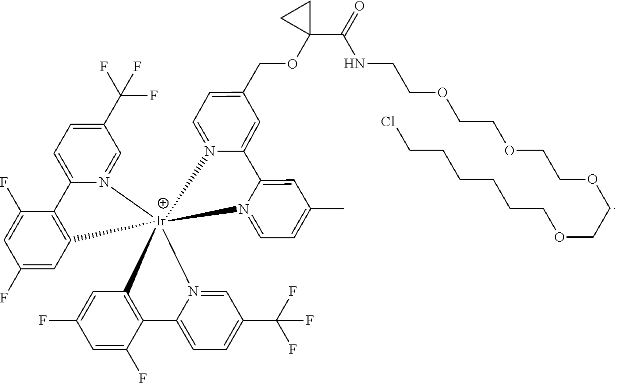

In some embodiments, the labeling agent has the structure:

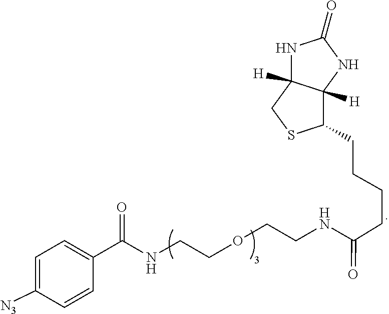

In another aspect, the present disclosure provides a protein complex, comprising the structure: P1-P2-Cat
where:
   P1 is a ubiquitin ligase;
   P2 is a haloalkane dehalogenase;
   Cat is a photocatalyst having the structure of Formula (I):

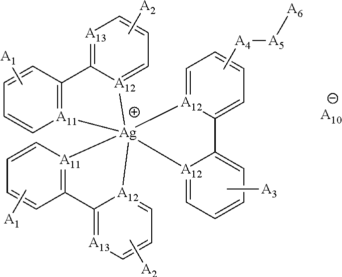

wherein:
   A1 is present 0-4 times on the ring to which it is attached and each A1 is independently selected from CH₃, CF₃, F, Cl, N(CH₃)₂, and OR₁;
   each R₁ is independently selected from H, a linear or branched alkyl group having 1-12 carbons, CHF₂, and CF₃;
   A2 is present 0-4 times on the ring to which it is attached and each A2 is independently selected from CH₃, CF₃, F, Cl, N(CH₃)₂, and OR₁;
   A3 is present 0-4 times on the ring to which it is attached and each A3 is independently selected from CH₃, CF₃, F, Cl, N(CH₃)₂, and OR₁;
   A4 is selected from null, linear or branched alkyl group having 1-12 carbons, C₆₋₁₀ aryl, C₃₋₈ cycloalkyl, 4-10 membered heterocyclyl, and 5-10 membered heteroaryl, wherein said alkyl, aryl, cycloalkyl, heterocyclyl, and heteroaryl is optionally substituted with one or more C₁₋₆ alkyl, C₁₋₆ haloalkyl, halo, hydroxy, C₁₋₆ alkoxy, or amino;
   A5 is selected from CONH, NHCO, SONH, SO₂NH, NHSO, NHSO₂, NH, OCONH, NHCOO, C₁₋₆ alkoxy, C₁₋₆ alkoxy, and

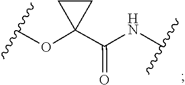 [SMILES: *NC(=O)C1(O*)CC1]

A6 is independently (PEG)a₁(CH₂)a₂C₁ or (PEG)a₁(CH₂)a₂(5-6 membered heterocyclyl)(PEG)a₁(CH₂)a₂Cl, wherein each a₁ is independently an integer from 0-10 and each a₂ is independently an integer from 6-10;
A9 is a D9 metal selected from copper, vanadium, chromium, scandium, titanium, manganese, iron, cobalt, nickel, copper, zinc, yttrium, zirconium, niobium, molybdenum, technetium, ruthenium, rhodium, palladium, silver, cadmium lanthanum, hafnium, tantalum, tungsten, rhenium, osmium, iridium, platinum, gold, and mercury;
A11 and A12 are independently a C or N coordinated to A9; and
each A13 is independently CH or N.

In some embodiments, A3 may be present 0-4 times on the ring to which it is attached and each A3 may be independently selected from CH₃, CF₃, F, Cl, N(CH₃)₂, and OR₁. In some embodiments, A5 may be selected from CONH, NHCO, SONH, SO₂NH, NHSO, NHSO₂, NH, OCONH, and NHCOO. In some embodiments, A6 may be (PEG)a₁(CH₂)a₂Cl, a₁ may be an integer from 0-10 and a₂ may be an integer from 6-10. In some embodiments, A13 may be CH. In some embodiments, the 5-6 membered heterocyclyl of A6 may be piperazine or pyrrolidine. In some embodiments, Cat has the structure:

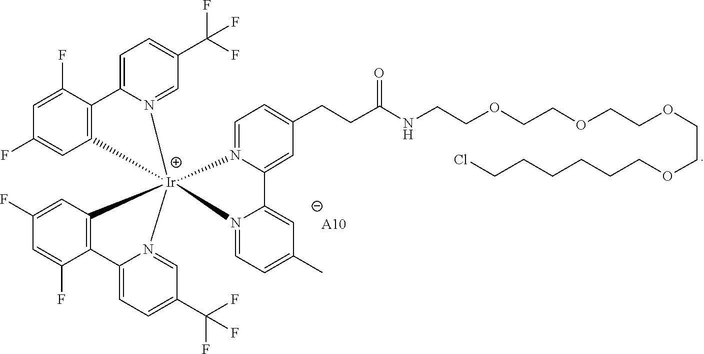

In some embodiments, Cat has the structure:

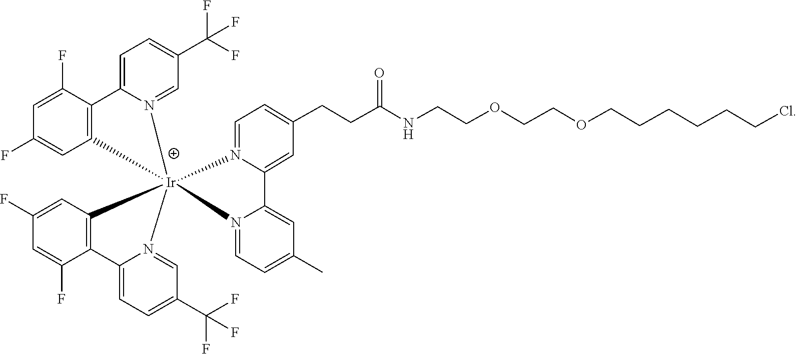

In some embodiments, Cat has the structure:

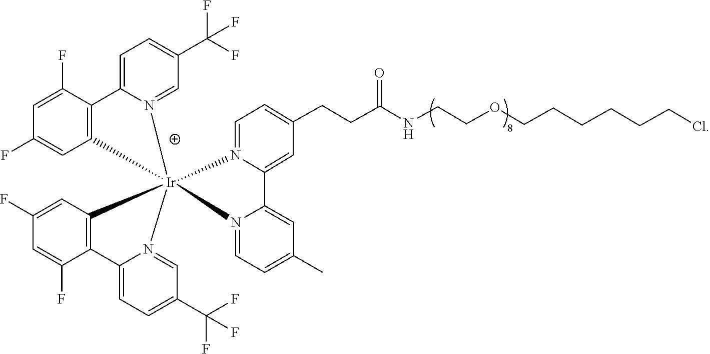

In some embodiments, Cat has the structure:

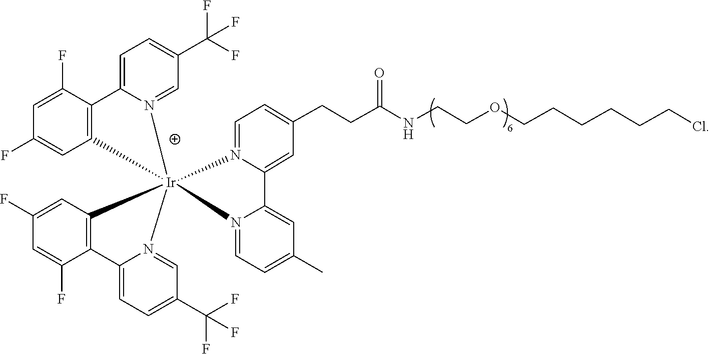

In some embodiments, Cat has the structure:

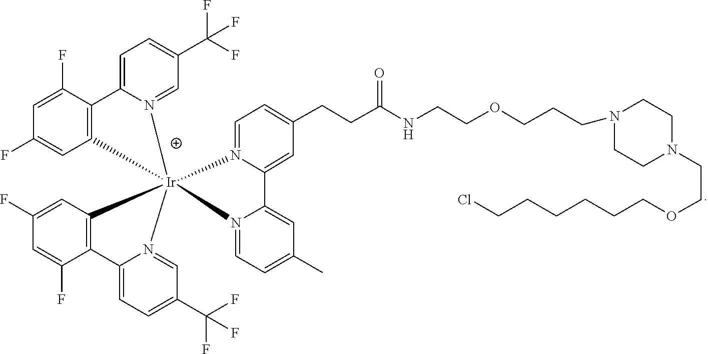

In some embodiments, Cat has the structure:

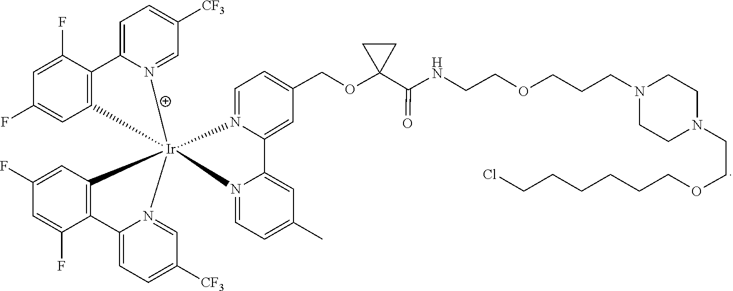

In some embodiments, Cat has the structure:

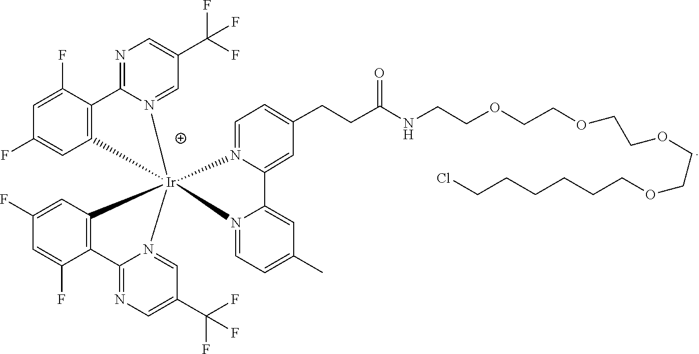

In some embodiments, Cat has the structure:

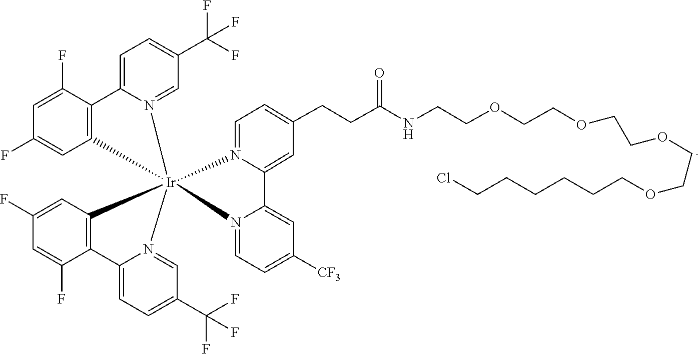

In some embodiments, Cat has the structure:

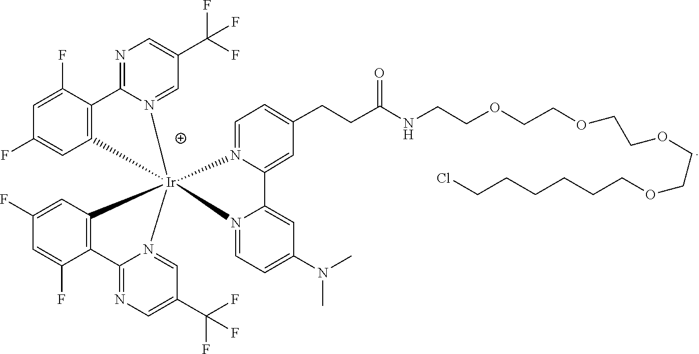

In some embodiments, Cat has the structure:

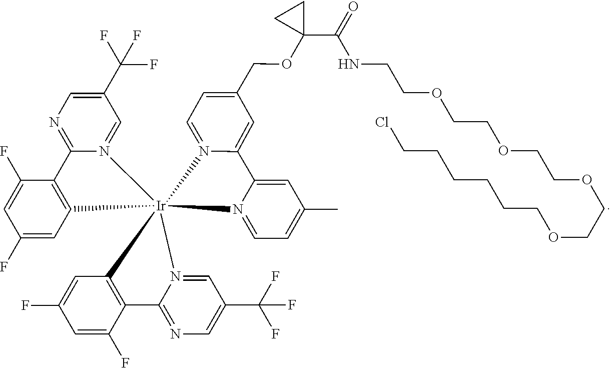

In another aspects, the present disclosure provides a cell, including:
a protein complex of in accordance with the present disclosure; and
a labeling agent having the structure of Formula (III-a):

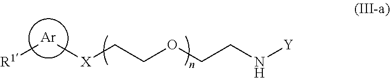

wherein:
R¹′ is selected from an azide, a methyl diazirine, a trifluoromethyl diazirine, and a phenyl diazirine;
Ring Ar is selected from phenyl, pyridyl, pyrimidyl, pyrazinyl, pyridizynyl, naphthyl, and quinolinyl, optionally substituted with one or more —OH, —OMe, —OEt, —OCF₃, —OCF₂H, —NHMe, —NMe₂, —F, —Cl, —Br, -Me, or -Et;
X is selected from 0, NH, NR², CH₂NHCO, CONH, CONR², SO₂NH, and SO₂NR²;
R² is selected from H, OMe, Me, and Et;
n is 0, 1, 2, 3, 4, 5, or 6; and
Y is a biotin-linked amide or an amide-linked fluorescent dye.

In another aspect, the present disclosure provides a cell, including a nucleotide sequence expressing a fusion protein comprising a ubiquitin ligase and a haloalkane dehalogenase.

In another aspect, the present disclosure provides a photocatalyst having the structure of Formula (I):

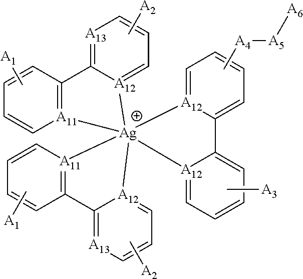

wherein:
   A1 is present 0-4 times on the ring to which it is attached and each A1 is independently selected from CH₃, CF₃, F, Cl, N(CH₃)₂, and OR₁;
   each R₁ is independently selected from H, a linear or branched alkyl group having 1-12 carbons, CHF₂, and CF₃;
   A2 is present 0-4 times on the ring to which it is attached and each A2 is independently selected from CH₃, CF₃, F, Cl, N(CH₃)₂, and OR₁;
   A3 is present 0-4 times on the ring to which it is attached and each A3 is independently selected from CH₃, CF₃, F, Cl, N(CH₃)₂, and OR₁;
   A4 is selected from null, linear or branched alkyl group having 1-12 carbons, C₆₋₁₀ aryl, C₃₋₈ cycloalkyl, 4-10 membered heterocyclyl, and 5-10 membered heteroaryl, wherein said alkyl, aryl, cycloalkyl, heterocyclyl, and heteroaryl is optionally substituted with one or more C₁₋₆ alkyl, C₁₋₆ haloalkyl, halo, hydroxy, C₁₋₆ alkoxy, or amino;
   A5 is selected from CONH, NHCO, SONH, SO₂NH, NHSO, NHSO₂, NH, OCONH, NHCOO, C₁₋₆ alkoxy, and

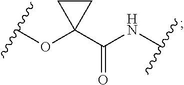 [SMILES: *NC(=O)C1(O*)CC1]

A6 is independently (PEG)a₁(CH₂)a₂Cl or (PEG)a₁(CH₂)a₂(5-6 membered heterocyclyl)(PEG)a₁(CH₂)a₂Cl, wherein each a₁ is independently an integer from 0-10 and each a₂ is independently an integer from 6-10;
A9 is a D9 metal selected from copper, vanadium, chromium, scandium, titanium, manganese, iron, cobalt, nickel, copper, zinc, yttrium, zirconium, niobium, molybdenum, technetium, ruthenium, rhodium, palladium, silver, cadmium lanthanum, hafnium, tantalum, tungsten, rhenium, osmium, iridium, platinum, gold, and mercury;
A11 and A12 are independently a C or N coordinated to A9;
each A13 is independently CH or N, and
wherein the photocatalyst is not

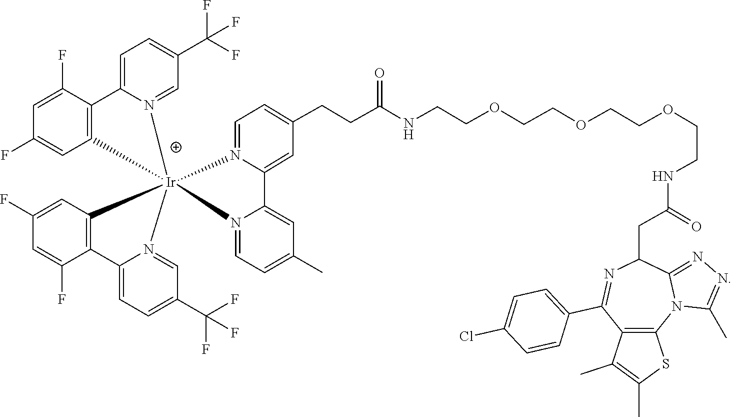

In some embodiments, A3 is present 0-4 times on the ring to which it is attached and each A3 is independently selected from CH₃, CF₃, F, Cl, N(CH₃)₂, and OR₁. In some embodiments, A5 is selected from CONH, NHCO, SONH, SO₂NH, NHSO, NHSO₂, NH, OCONH, and NHCOO. In some embodiments, A6 is (PEG)a₁(CH₂)a₂Cl, wherein a₁ is an integer from 0-10 and a₂ is an integer from 6-10. In some embodiments, A13 is CH. In some embodiments, the 5-6 membered heterocyclyl of A6 is piperazine or pyrrolidine. In some embodiments, the photocatalyst has the structure:

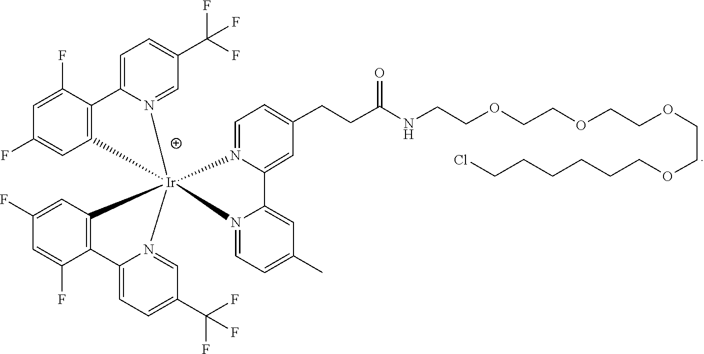

In some embodiments, the photocatalyst has the structure:

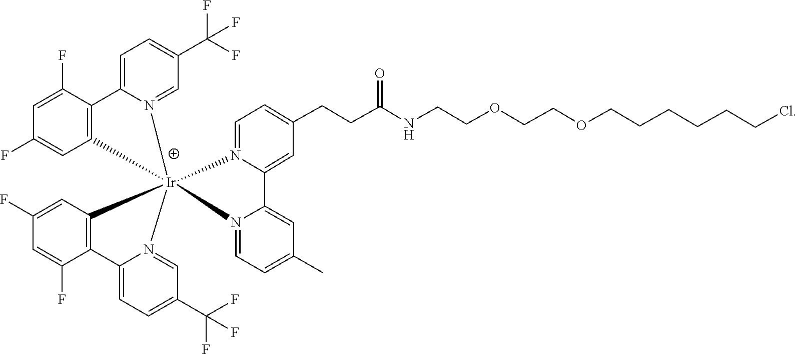

In some embodiments, the photocatalyst has the structure:

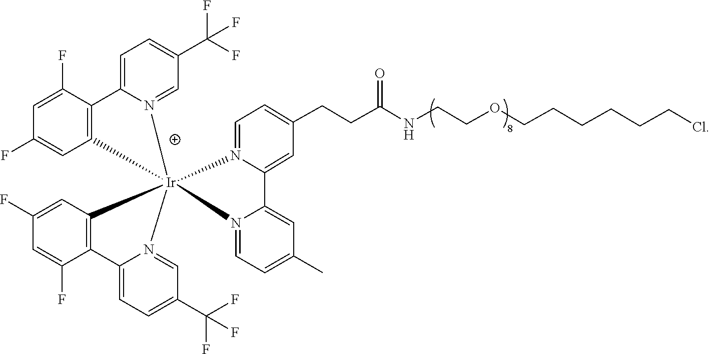

In some embodiments, the photocatalyst has the structure:

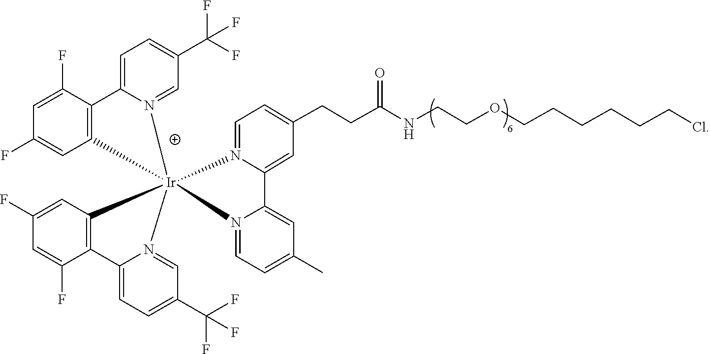

In some embodiments, the photocatalyst has the structure:

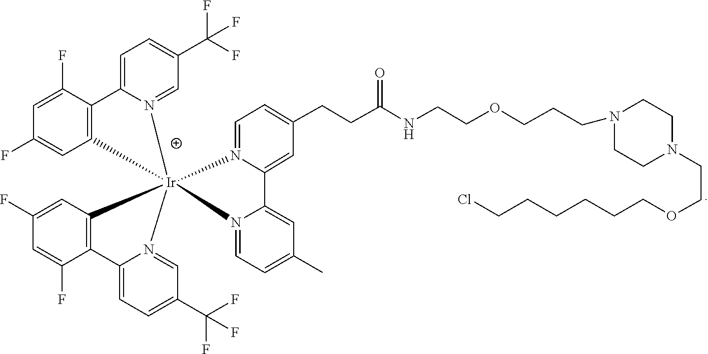

In some embodiments, the photocatalyst has the structure:

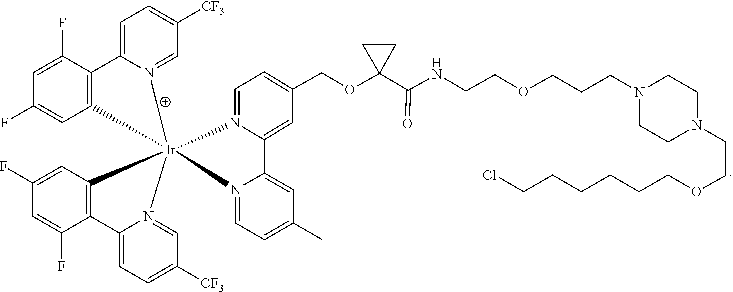

In some embodiments, the photocatalyst has the structure:

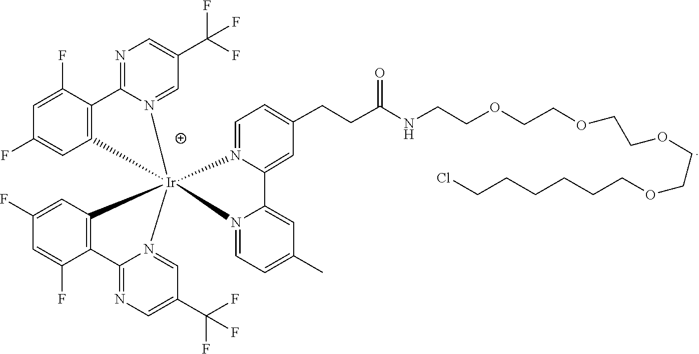

In some embodiments, the photocatalyst has the structure:

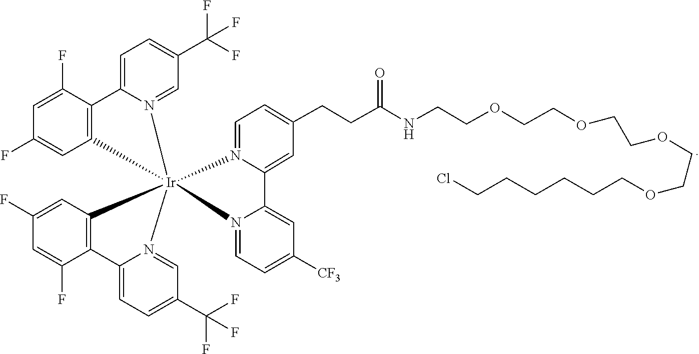

In some embodiments, the photocatalyst has the structure:

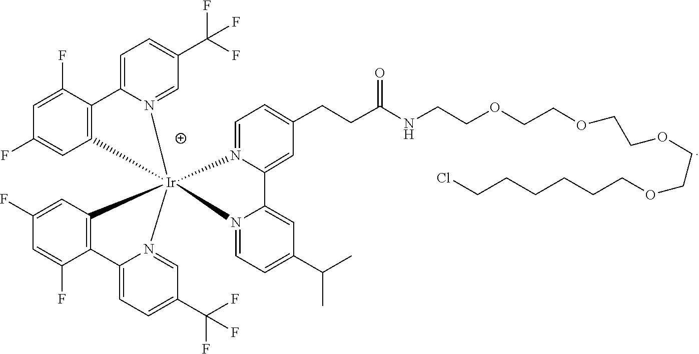

In some embodiments, the photocatalyst has the structure:

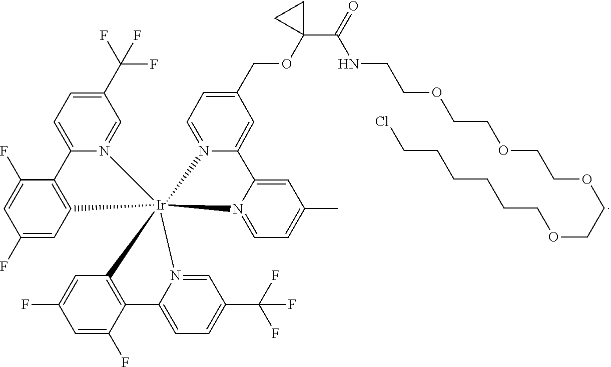

### BRIEF DESCRIPTION OF THE DRAWINGS

Throughout the drawings, reference numbers are re-used to indicate correspondence between referenced elements. The following drawings and the associated descriptions are provided to illustrate implementations of the present disclosure and do not limit the scope of the claims.

FIG. 1 is a diagram showing the formation of a catalyst-antenna assembly in accordance with the present disclosure.

FIG. 2 is a diagram of photoactivation of a photocatalyst which subsequently activates a labeling agent via energy transfer such that a label is bound to a proximate biomolecule.

FIG. 3 is a flow chart illustrating the process of labeling a target intracellular biomolecule in accordance with the present disclosure.

FIGS. 4A-4C are images of Western blot results of the procedure described in Example 5, involving the immunoprecipitation of HALO-tagged proteins.

FIGS. 5A-5F are images of Western blot results of the procedure described in Example 6, involving the immunoprecipitation biotinylated proteins.

FIG. 6 is an image of Western blot results indicating that a HaloTag-cereblon fusion protein is capable of ubiquitinating ikaros.

FIGS. 7A-7B are images of Western blot results indicating that CK1 was proximally labeled by a HaloTag-cereblon fusion protein.

FIGS. 8A-8B are images of Western blot results indicating that SALL4 was proximally labeled by a HaloTag-cereblon fusion protein.

FIG. 9 is an image of Western blot results indicating that KEAP1 was not labeled by a HaloTag-cereblon fusion protein.

FIG. 10 is an image of Western blot results indicating that β-tubulin was not labeled by a HaloTag-cereblon fusion protein.

FIGS. 11A-11B are images of Western blot results indicating that PARP1 was not labeled by a HaloTag-cereblon fusion protein.

FIGS. 12A-12B are images of Western blot results indicating that a known oncogenic protein was labeled by a HaloTag-cereblon fusion protein.

FIGS. 13A-13B are images of Western blot results indicating that IKZF1 was labeled by a HaloTag-cereblon fusion protein.

FIGS. 14A-14B are images of Western blot results indicating that cRAF was labeled by both an immunoprecipitation pull-down approach and a HaloTag-based proximity labeling approach.

FIGS. 15A-15B are images of Western blot results indicating that RSK1 was labeled by a HaloTag-based proximity labeling approach, but not by an immunoprecipitation pull-down approach.

FIGS. 16A-16B are images of Western blot results indicating that SOS1 was labeled by a HaloTag-based proximity labeling approach, but not by an immunoprecipitation pull-down approach.

FIGS. 17A-17B are images of Western blot results indicating that KEAP1 was labeled by neither an immunoprecipitation pull-down approach nor a HaloTag-based proximity labeling approach.

FIGS. 18A-18H are images of Western blot results demonstrating the intracellular labeling of K-Ras interactor by a HaloTag-KRAS protein may be photo-irradiation time dependent.

FIGS. 19A-19B are images of Western blot results demonstrating photocatalytic biotinylation of bovine serum albumin (BSA) by using a diazirine biotin conjugate with different Ir-photocatalysts.

FIGS. 20A-20B are images of Western blot results demonstrating photocatalytic biotinylation of bovine serum albumin (BSA) by using a diazirine biotin conjugate with different Ir-photocatalysts.

FIGS. 21A-21B are images of Western blot results demonstrating cell permeability of different Ir-photocatalysts by Halotag chaser assay.

### DETAILED DESCRIPTION

The present disclosure will now be described with reference to the accompanying figures, wherein like numerals refer to like elements throughout. The following description is merely illustrative in nature and is in no way intended to limit the disclosure, its application, or uses. It should be understood that steps within a method may be executed in different order without altering the principles of the present disclosure. Furthermore, implementations disclosed herein can include several novel features, no single one of which is solely responsible for its desirable attributes or which is essential to practicing the systems, devices, and methods disclosed herein.

Systems, compositions, and methods disclosed herein relate to techniques to label intracellular molecules within proximity of a protein of interest. Such labeling may allow for in vitro and in vivo proximity-based protein labeling. Labeling may be accomplished by a photo-responsive catalyst coupled to a protein of interest to activate a cell-permeable labeling agent via Dexter energy transfer, which can then label a proximate biomolecule. Specifically, when activated, the photo-responsive catalyst will activate a reactive moiety on the cell-permeable labeling agent. That reactive moiety can then bind with various biomolecules near the protein of interest. A label on the labeling agent can then be detected, such as by imaging, to provide information regarding the intracellular locations of the protein of interest.

More specifically, disclosed herein are methods and systems by which self-labeling systems, such as HaloTag, can be utilized to incorporate a photo-responsive catalyst, such as iridium, into a living system for microenvironment mapping. In accordance with the present disclosure, when used in concert with an appropriate cell-permeable labeling agent, photo-responsive catalysts can harness incident photonic energy and transfer said energy to activate a cell-permeable labeling agent to label constituents of a biomolecular microenvironment.

### System and Compounds for Intracellular Proximity-Based Labeling

In one aspect, a system for proximity-based labeling of intracellular molecules includes: a cell; a biomolecular antenna including a photocatalyst complex further including a photocatalyst and a ligand moiety, and a binding agent complex, including a protein of interest coupled to a binding agent, wherein the binding agent is capable of binding the ligand moiety of the photocatalyst complex; and a labeling agent including a label moiety and a reactive moiety, where the reactive moiety is configured to be activated to a reactive state by the photocatalyst complex; where the biomolecular antenna and the labeling agent are each located within the cell.

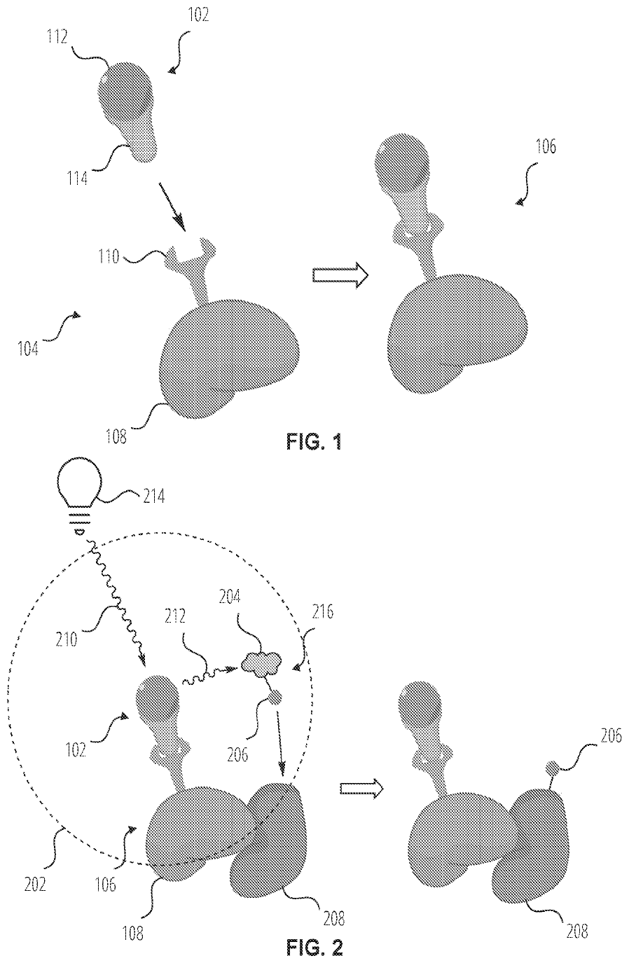

FIG. 1 is a drawing showing formation of a biomolecular antenna 106 of an example intracellular proximity-based labeling system. The biomolecular antenna 106 includes a catalyst complex 102 and a binding agent complex 104. The catalyst complex 102, in turn, includes a photocatalyst 112 and a ligand moiety 114. The binding agent complex 104 includes a protein of interest 108 and a binding agent 110. The binding agent 110 may be capable of binding the ligand moiety 114 of the catalyst complex 102 such that the catalyst complex 102 and the binding agent complex 104 are capable of forming the biomolecular antenna 106.

The catalyst complex 102 may be any suitable catalyst complex 102 for activating a labeling agent 216 (described below) in accordance with the present embodiment. In some embodiments, the catalyst complex 102 may include a transition metal complex including a transition metal. In these embodiments, the transition metal complex may include a ligand or ligands coordinated to a metal center. In certain embodiments, the transition metal may be a platinum group metal. In certain embodiments, the transition metal may be iridium or ruthenium. In some embodiments, the transition metal may have a triplet energy state greater than 1 kcal/mol, greater than 5 kcal/mol, greater than 10 kcal/mol, greater than 20 kcal/mol, greater than 30 kcal/mol, greater than 40 kcal/mol, greater than 50 kcal/mol, greater than 60 kcal/mol, greater than 75 kcal/mol, greater than 100 kcal/mol, greater than 150 kcal/mol, greater than 200 kcal/mol, greater than 250 kcal/mol, greater than 500 kcal/mol, greater than 1000 kcal/mol, or any value or range within or bounded by any of these ranges or values, although values outside these values or ranges can be used in some cases.

In some embodiments, the transition metal (also referred to herein as a transition metal catalyst) may be capable of absorbing one or more wavelengths of visible light, (i.e., light having a wavelength within about 380 nm to about 700 nm, although values outside this range can be used in some cases). Absorption of visible light can excite the transition metal complex to the S1 state followed by quantitative intersystem crossing to a long-lived triplet excited state (T1). The T1 state may have half-life t_{1/2} of between 50 ns-5 μs, between 0.1 μs-2.5 μs, or between 0.2 μs-2 μs, or any value or range within or bounded by any of these ranges or values, although values outside these values or ranges can be used in some cases. In some embodiments, the transition metal catalyst can undergo short-range Dexter energy transfer to a labeling agent and return to the ground state S0. The energy transfer to the labeling agent may activate the labeling agent for reaction with a protein or other biomolecule. If the labeling agent is a diazirine, as an illustrative example, triplet energy transfer to the labeling agent can form a carbene intermediate for reacting with a protein or other biomolecular species.

The photocatalytic transition metal complex can have any composition and structure consistent with the foregoing principles and energy transfer. In some embodiments, the photocatalytic transition metal complex is hexacoordinate. The ligand moiety 114 may include one or more ligands capable of binding with the binding agent 110. Ligands of the ligand moiety 114 of the catalyst complex 102, for example the transition metal complex, can include one or more pyridine moieties, in some embodiments. In some embodiments, one or more ligands include bipyridine or derivatives thereof. Table I provides examples of suitable ligands:

TABLE I
Example photocatalytic transition metal complex ligands

4,4′-di-tert-butyl-2,2′-dipyridyl (dtbbpy)
2,2′-bipyridine (bpy)
diphenhydramine-2,2′-bipyridine (diPh-bpy)
4-4′-dimethoxy-2-2′-bipyridine (diOMe-bpy)
dinapthalene-pyrene (di-naphth-Py)
phenanthroline (phen)
diphenyl-phenanthroline (di-Ph-Phen)

In some embodiments, the photocatalytic transition metal complex is [Ir(dF(CF₃)ppy)₂(dtbbpy)](PF₆), ([Ir(dF(CF₃)ppy)₂(bpy)](PF₆)), or derivatives thereof. In some embodiments, an iridium photocatalyst may be used as described in Trowbridge et al., Small molecule photocatalysis enables drug target identification via energy transfer, bioRxiv (2021) https://doi.org/10.1101/2021.08.02.454797, incorporated herein by reference.

In some embodiments, the photocatalytic transition metal complex may have a structure of Formula I:

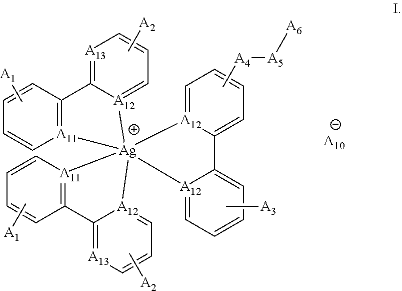

where A1 may be 0-4 substituents on the ring to which it is attached and each A1 may independently be CH₃, CF₃, F, Cl, N(CH₃)₂, or OR₁;
R₁ may independently be H, a linear or branched alkyl group having 1-12 carbons, CHF₂, CF₃;
A2 may be 0-4 substituents on the ring to which it is attached selected from CH₃, CF₃, F, Cl, N(CH₃)₂, or OR₁;
A3 may be 0-4 substituents on the ring to which it is attached selected from CH₃, CF₃, F, Cl, N(CH₃)₂, or OR₁;
A4 may be null, linear or branched alkyl group having 1-12 carbons, C₆₋₁₀ aryl, C₃₋₈ cycloalkyl, 4-10 membered heterocyclyl, and 5-10 membered heteroaryl, wherein said alkyl, aryl, cycloalkyl, heterocyclyl, and heteroaryl is optionally substituted with one or more C₁₋₆ alkyl, C₁₋₆ haloalkyl, halo, hydroxy, C₁₋₆ alkoxy, or amino
A5 may be CONH, NHCO, SONH, SO₂NH, NHSO, NHSO₂, NH, OCONH, NHCOO, C₁₋₆ alkoxy, and

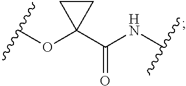 [SMILES: *NC(=O)C1(O*)CC1]

A6 may be independently (PEG)a₁(CH₂)a₂C₁ or (PEG)a₁(CH₂)a₂(5-6 membered heterocyclyl)(PEG)a₁(CH₂)a₂Cl, where, for example, a₁ may be defined as the number of polyethylene glycol (PEG) subunits with a₁ being an integer from 0-10 and a₂ may be defined as the number of methylene subunits with a₂ being an integer from 6-10, in some examples the 5-6 membered heterocyclyl of A6 may be a piperazine or a pyrrolidine;
A9 may be a D9 metal, for example copper, vanadium, chromium, scandium, titanium, manganese, iron, cobalt, nickel, copper, zinc, yttrium, zirconium, niobium, molybdenum, technetium, ruthenium, rhodium, palladium, silver, cadmium lanthanum, hafnium, tantalum, tungsten, rhenium, osmium, iridium, platinum, gold, and mercury;
A10 may be an anion to the metal of A9; a non-exclusive list of such anions includes tetraalkylborate, tetrafluoroborate, tetraphenylborate, chloride, cyanide, hexafluorophosphate, bis(triphenylphosphine)iminium chloride, tetraphenylphosphonium Chloride, or tetrabutylammonium; and
A11 and A12 may be independently a C or N coordinated to A9; and
each A13 is independently CH or N.

In some embodiments, A1 may be F. In some embodiments, A1 may be Cl. In some embodiments, A1 may be CH₃. In some embodiments, A1 may be CF₃. In some embodiments, A1 may be N(CH₃)₂. In some embodiments, A1 may be OR₁. In some embodiments, A1 may be present 1 time on the ring to which it is attached. In some embodiments, A1 may be present 2 times on the ring to which it is attached. In some embodiments, A1 may be present 2 times on the ring to which it is attached and may be a F. In some embodiments, A1 may be present 1 time on the ring to which it is attached and may be a F. In some embodiments, A1 may be present 2 times on the ring to which it is attached and may be a Cl. In some embodiments, A1 may be present 1 time on the ring to which it is attached and may be a Cl. In some embodiments, A1 may be present 2 times on the ring to which it is attached and may be a CH₃. In some embodiments, A1 may be present 1 time on the ring to which it is attached and may be a CH₃. In some embodiments, A1 may be present 2 times on the ring to which it is attached and may be a CF₃. In some embodiments, A1 may be present 1 time on the ring to which it is attached and may be a CF₃. In some embodiments, A1 may be present 2 times on the ring to which it is attached and may be a N(CH₃)₂. In some embodiments, A1 may be present 1 time on the ring to which it is attached and may be a N(CH₃)₂. In some embodiments, A1 may be present 2 times on the ring to which it is attached and may be a OR₁. In some embodiments, A1 may be present 1 time on the ring to which it is attached and may be a OR₁. In some embodiments, A2 may be F. In some embodiments, A2 may be Cl. In some embodiments, A2 may be CH₃. In some embodiments, A2 may be CF₃. In some embodiments, A2 may be N(CH₃)₂. In some embodiments. A2 may be OR₁. In some embodiments, A2 may be present 1 time on the ring to which it is attached. In some embodiments, A2 may be present 2 times on the ring to which it is attached. In some embodiments, A2 may be present 2 times on the ring to which it is attached and may be a F. In some embodiments, A2 may be present 1 time on the ring to which it is attached and may be a F. In some embodiments, A2 may be present 2 times on the ring to which it is attached and may be a Cl. In some embodiments, A2 may be present 1 time on the ring to which it is attached and may be a Cl. In some embodiments, A2 may be present 2 times on the ring to which it is attached and may be a CH₃. In some embodiments, A2 may be present 1 time on the ring to which it is attached and may be a CH₃. In some embodiments, A2 may be present 2 times on the ring to which it is attached and may be a CF₃. In some embodiments, A2 may be present 1 time on the ring to which it is attached and may be a CF₃. In some embodiments, A2 may be present 2 times on the ring to which it is attached and may be a N(CH₃)₂. In some embodiments, A2 may be present 1 time on the ring to which it is attached and may be a N(CH₃)₂. In some embodiments, A2 may be present 2 times on the ring to which it is attached and may be a OR₁. In some embodiments, A2 may be present 1 time on the ring to which it is attached and may be a OR₁. In some embodiments, A11 may be a C coordinated to A9. In some embodiments, A12 may be a N coordinated to A9. In some embodiments, In some embodiments In some embodiments, one or more of the ligands can include hydrophilic moieties for enhancing compatibility of the transition metal complex with water or aqueous-based environments, such as those found within a cell. Additionally, one or more of the ligands may include a moiety, functionality, and/or handle for coupling a binding agent 110, such as a protein, polysaccharide or nucleic acid. For example, a moiety including an appropriately long alkyl chloride, for example an alkyl chloride having 6 or more carbons, can be bound by binding agent 110 that includes a halodehalogenase (e.g., a HaloTag) that is genetically encoded to be produced with the protein of interest 108. In certain embodiments, the photocatalytic transition metal complex has the structure of Formula II-XI:

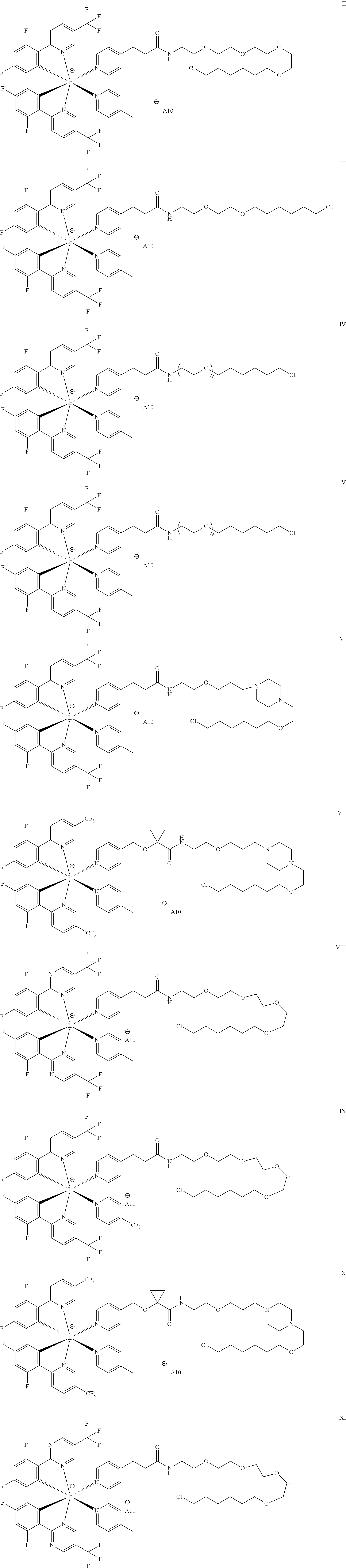

In some embodiments, the transition metal may have a visible light extinction coefficient greater than about 10 M⁻¹cm⁻¹, greater than about 100 M⁻¹ cm⁻¹, greater than about 250 M⁻¹ cm⁻¹, greater than about 500 M⁻¹cm⁻¹, greater than about 1000 M⁻¹ cm⁻¹, greater than about 2000 M⁻¹ cm⁻¹, greater than about 5000 M⁻¹ cm¹, greater than about 10,000 M⁻¹cm⁻¹, or any value or range within or bounded by any of these ranges or values, although values outside these values or ranges can be used in some cases.

In some embodiments, the photocatalyst 112 of the catalyst complex 102 is an organocatalyst. In some embodiments, such an organocatalyst may not include a photo-responsive transition metal. As illustrative examples of such embodiments, the organocatalyst may include a thioxanthone group, phenothiazine group, flavin group, phenoxazine group, pthalazine group, quinoxaline group, quinazoline group, benzophenothiazine group, coumarin group, acetophenone group, or benzophenone group. However, any suitable organocatalyst capable of activating a labeling agent 216 in accordance with the present disclosure may be selected.

The protein of interest 108 may be a suitable intracellular molecule. For example, the protein of interest 108 may be implicated in a signaling pathway, a subcellular and/or cellular architecture, a localized biomolecular network, and/or a microenvironment. In some embodiments, the protein of interest 108 may be encoded for by a nucleotide construct introduced to and expressed by the cell. In other embodiments, the protein of interest 108 may be permeable to the cell. In yet other embodiments, the protein of interest 108 may not be permeable to the cell but may be introduced by to the cell by some suitable method, for example by depositing via liposome. In various embodiments, the protein of interest may be involved in a disease state. As illustrative examples, proteins involved in cancer include K-Ras, cMyc, Src, WRN, Slug, and PARP1. As illustrative examples, proteins involved in neurodegeneration include Aβ and Tau. As illustrative examples, proteins involved in viral infection may include influenza hemagglutinin and viral nucleoprotein. In some embodiments, the protein of interest 108 is a ubiquitin ligase (e.g., an E3 ligase).

The binding agent 110 may be any suitable molecule having specific binding to the ligand moiety 114. In some embodiments, the binding agent 110 may include a haloalkane dehalogenase. In certain embodiments, the binding agent 110 may be a HaloTag®. The HaloTag® (Promega) purification system has been developed as an engineered halodehalogenase protein which can be produced natively in cells. The HaloTag system can be used in a number of self-labeling processes for chemical biology. As illustrative examples, the HaloTag system can be used for protein purification and incorporation of TAMRA-alkyl chloride dyes for the monitoring of intracellular signaling. Because halodehalogenase is non-mammalian, there should theoretically be low, substantially none, or no background incorporation of the photo-responsive catalyst via a dehalogenase mechanism. As such, the HaloTag system can serve as a useful vector to incorporate the photo-responsive catalysts into a native cellular environment for the identification of the microenvironment interactome of a protein of interest. The HaloTag system may thus be useful for monitoring transient interactions in the intracellular environment.

In some embodiments, a nucleotide construct that encodes for a fusion of the protein of interest 108 and the binding agent 110 (i.e., a binding agent complex 104) may be introduced to the cell. The cell may express the binding agent complex 104 encoded by the construct. In some embodiments, the construct may encode for a binding agent complex 104 that is a fusion protein, where the binding agent 110 is attached to the C-terminus of the protein of interest 108. In some embodiments, the construct may encode for a binding agent complex 104 that is a fusion protein, where the binding agent 110 is attached to the N-terminus of the protein of interest 108. In some embodiments, the construct may encode for a binding agent complex 104 where the binding agent 110 is attached to the protein of interest 108 at a site where the binding agent 110 does not disrupt, does not substantially disrupt, or minimally disrupts certain interactions of the protein of interest 108 with biomolecules 208.

FIG. 2 is a drawing of a scheme for proximity-based labeling including the biomolecular antenna 106 illustrated in FIG. 1. Additionally included is a labeling agent 216, which includes a reactive moiety 204 and a label moiety 206. A light source 214 may generate photons 210 which activate the catalyst complex 102. In turn, the catalyst complex 102 may activate the labeling agent 216 via Dexter energy transfer 212, activating the reactive moiety 204. When in an activated state, the labeling agent 216 may be capable of binding a label moiety 206 to a nearby biomolecule 208.

In accordance with the present disclosure, the labeling agent 216 may be activated via Dexter energy transfer 212. Although diazirine and azide-based probes have been widely applied in small molecule target-ID, they require direct excitation with UV light, thereby precluding the possibility of a target-localized activation. It is known that these types of reactive moieties have the capacity to receive triplet energy via a Dexter energy transfer. Attaching a photocatalyst (e.g., a photocatalyst 112 of a catalyst complex 102) to a cellularly-produced biomolecule (e.g., a protein of interest 108) allows it to act as an antenna, absorbing the photonic energy of light, for example visible light. The photocatalyst 112 is activated by absorbing light. The activated photocatalyst 112 can then activate a labeling agent 216 within the immediate vicinity of the protein of interest 108 via Dexter energy transfer 212. The activated labeling agent 216 can thereby covalently attach itself to molecular structures (including, but not limited to, proteins, chromatin, and nucleic acids) in the immediate vicinity of the binding agent complex 104 through the activated reactive moiety 204. The distance over which the activated labeling agent 216 is capable of attaching a label moiety 206 is dependent on the diffusivity of labeling agent 216 and the length of the activated half-life t_{1/2} of reactive moiety 204. For example, the longer the activated half-life t_{1/2} of reactive moiety 204, the greater the length of time the labeling agent 216 may travel before attaching to a biomolecule 208. Similarly, the greater the diffusivity of the labeling agent 216, the larger the distance the labeling agent 216 will be able to travel in an activated state before quenching or binding to a biomolecule 208.

In embodiments where the labeling agent 216 includes a reactive moiety 204 attached to a label moiety 206, the attachment of reactive moiety 204 to a biomolecule 208 also results in the label moiety 206 being transferred to the biomolecule 208 (including, but not limited to, proteins, chromatin, and nucleic acids). Placement of a label on the biomolecule 208 enables orthogonal technologies (including, but not limited to, proteomic analysis, flow cytometry, radiographic imaging and electron microscopy) to be used to identify the nature, identity and spatiotemporal aspects of the microenvironment. In some embodiments, the reactive moiety 204 may be cell-permeable, with or without an attached label moiety 206. In some embodiments, the labeling agent 216 is cell-permeable.

In some embodiments, a labeling agent 216 may have a structure as depicted in Formula III:

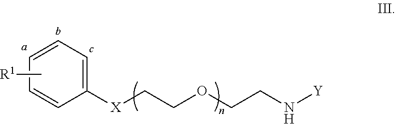

where R¹ may be a substituent capable of accepting triplet energy from an activated photocatalyst. For example, R¹ may be an azide, a methyl diazirine, a trifluoromethyl diazirine, or a phenyl diazirine;
R¹ may be positioned independently on the aromatic ring at positions a, b, or c;
the aromatic ring may be phenyl, pyridyl, pyrimidyl, pyrazinyl, or pyridizynyl.

The aromatic ring may be naphthyl or quinoline and may include various additional connections,
the aromatic ring can be substituted with electron withdrawing or donating groups to attenuate the reactivity of the labeling species. As illustrative examples, the substituents at positions a, b, or c may be —OH, —OMe, —OEt, —OCF₃, —OCF₂H, —NHMe, —NMe₂, —F, —Cl, —Br, -Me, -Et;
X may be O, NH, NR², CH₂NHCO, CONH, CONR², SO₂NH, SO₂NR²;
further, R² may be H, OMe, Me, Et
n is an integer indicating the number of PEG units; for example, n may be 0, 1, 2, 3, 4, 5, or 6, though in some cases other values of n may be used;
Y may be a biotin-linked amide, or an amide derived via ligation with a fluorescent dye.

In some embodiments, the labeling agent 216 has the structure of Formula (III-a):

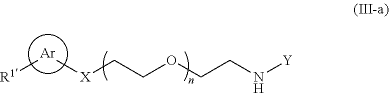

wherein:
R¹′ is selected from an azide, a methyl diazirine, a trifluoromethyl diazirine, and a phenyl diazirine;
Ring Ar is selected from phenyl, pyridyl, pyrimidyl, pyrazinyl, pyridizynyl, naphthyl, and quinolinyl, optionally substituted with one or more —OH, —OMe, —OEt, —OCF₃, —OCF₂H, —NHMe, —NMe₂, —F, —Cl, —Br, -Me, or -Et;
X is selected from O, NH, NR², CH₂NHCO, CONH, CONR², SO₂NH, and SO₂NR²;
R² is selected from H, OMe, Me, and Et;
n is 0, 1, 2, 3, 4, 5, or 6; and
Y is a biotin-linked amide or an amide-linked fluorescent dye.

The reactive moiety 204 can include any chemical species operable to interact with the photocatalyst 112 to form a reactive intermediate (also referred to herein as an activated labeling agent) for coupling to a biomolecule. As illustrative examples, in embodiments where the photocatalyst 112 includes a photocatalytic transition metal, the reactive moiety 204 may be an aryl azide or an alkyl azide. In such embodiments, triplet energy transfer from the excited-state photocatalyst 112 can promote the azide to its triplet (T1) state. The azide triplet undergoes elimination of N₂ to release a free triplet Nitrene, which undergoes picosecond-timescale spin equilibration to its reactive singlet state (with half-life of t_{1/2}<1 ns), resulting in either a crosslink with a nearby biomolecule 208 or quenching by the aqueous environment.

In certain embodiments, the labeling agent 216 may include a structure depicted in one of Formulas II-IX:

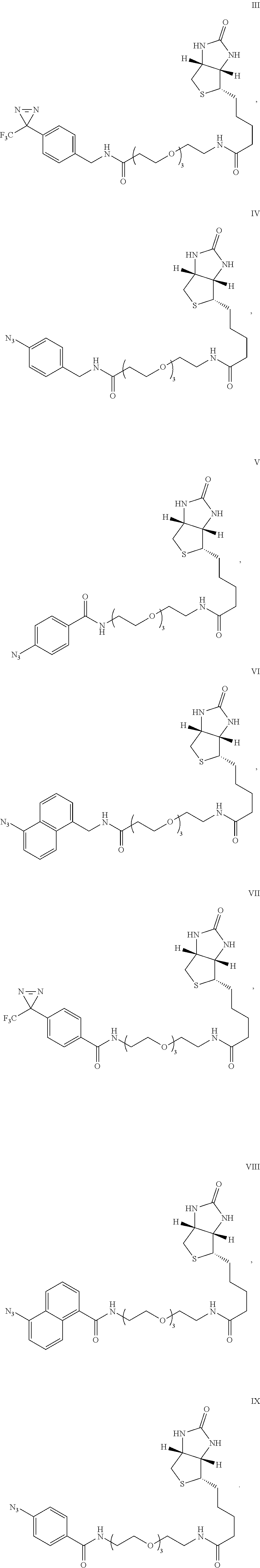

Azide sensitization could be extended to a variety of substitutions on the attached ring bearing valuable payloads (e.g., labels) for microscopy and proteomics applications. Such payloads or labels include free carboxylic acid, phenol, amine, alkyne, carbohydrate, and biotin groups.

The extinction coefficient of the photocatalyst 112 may be orders of magnitude larger than the extinction coefficient of the azide at the wavelength emitted by, for example, a blue light (e.g., light of wavelength about 450 nm) used for sensitization, ensuring no, substantially no, or minimal background non-catalyzed reaction of the reactive moiety 204. In other embodiments where the reactive moiety 204 may be activated to a singlet or triplet energy state in the absence of the photocatalyst, it may be desirable for the rate of labeling in the presence of the protein photocatalyst to be faster than that of the background reaction.

In some embodiments, a reactive intermediate (also referred to herein as an activated labeling agent) is formed via interaction of the reactive moiety 204 and the photocatalyst 112. The interaction may be an energy transfer, such as a Dexter energy transfer. The reactive intermediate can have t_{1/2} less than 50 ns, less than 10 ns, less than 5 ns, less than 2 ns, or less than 1 ns, or any value or range within or bounded by any of these ranges or values, although values outside these values or ranges can be used in some cases. In some embodiments, the reactive intermediate can have a half-life and diffusion constant such that the reactive intermediate diffuses less than 100 nm prior to quenching, less than 75 nm prior to quenching, less than 50 nm prior to quenching, less than 40 nm prior to quenching, less than 30 nm prior to quenching, less than 25 nm prior to quenching, less than 20 nm prior to quenching, less than 15 nm prior to quenching, less than 12.5 nm prior to quenching, less than 10 nm prior to quenching, less than 7.5 nm prior to quenching, less than 6 nm prior to quenching, less than 5 nm prior to quenching, less than 4 nm prior to quenching, less than 3 nm prior to quenching, less than 2 nm prior to quenching, or less than 1 nm prior to quenching, or any value or range within or bounded by any of these ranges or values, although values outside these values or ranges can be used in some cases. The foregoing mechanistic principles may be applied to proximity labeling of various types of biomolecules, including but not limited to proteins, polysaccharides, and/or nucleic acids.

In some embodiments, a reactive moiety 204 can be functionalized with a label moiety 206. As discussed, the labeling of a biomolecule 208 may facilitate the usage of orthogonal technologies. For example, in some embodiments, the label moiety 206 may include a tag capable of acting as a ligand for a tagging protein. In a subset of those embodiments, the label moiety 206 may include a biotin, which may bind to a tagging protein such as an avidin. Such tagging proteins may, as an illustrative example, be used in an immunoprecipitation assay to isolate the biomolecule 208 to which the label moiety 206 is attached. As another illustrative example, the tagging protein may include a fluorophore, such that the tagging protein may be imaged. In some embodiments, the label moiety 206 itself may include a fluorophore, such that it may be imaged without the addition of a tagging protein.

### Method of Proximity-Based Labeling

In one aspect, the present disclosure provides for a method of proximity-based labeling of intracellular molecules, including: introducing binding agent complex, where the binding agent complex includes a protein of interest coupled to a binding agent, where the binding agent is capable of binding to a catalyst complex; introducing the catalyst complex to the cell; forming a biomolecular antenna comprising the catalyst complex and the binding agent complex; introducing a labeling agent including a label moiety and a reactive moiety to the cell, wherein the reactive moiety is coupled to the label moiety; activating the catalyst complex, thereby activating the labeling agent by transfer of energy from the catalyst complex to the reactive moiety and causing the labeling agent to bind to a biomolecule within the cell.

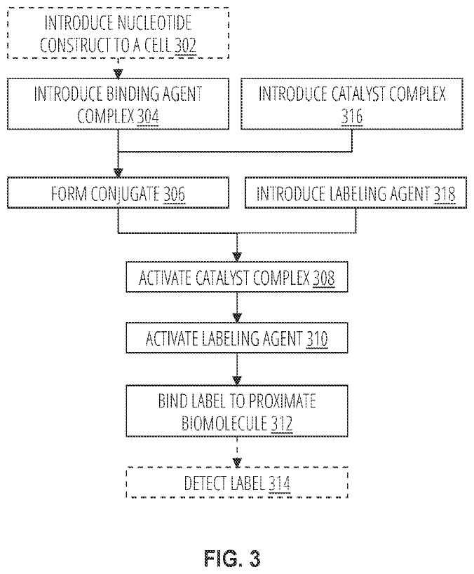

FIG. 3 diagrams a flowchart of a method in accordance with the present disclosure. A nucleotide construct may optionally be introduced to a cell, per block 302. In some embodiments, the nucleotide construct may be introduced via transformation and/or transfection. The nucleotide may encode for a binding agent complex 104 including a protein of interest 108 and a binding agent 110. The construct may be configured such that the portion encoding the binding agent complex 104 is expressible by the cell (e.g., the encoding portion may be within an open reading frame).

Then, per block 304, the binding agent complex 104 may be introduced to the cell. In embodiments where a nucleotide construct was introduced to the cell per block 302, the binding agent complex 104 may be introduced via the cell's expression of the nucleotide construct. Such production of the binding agent complex 104 may be intracellular, using the cell's own transcription and/or translation processes to produce the binding agent complex 104 based on the sequences found in the nucleotide construct. In other embodiments, the binding agent complex104 may be introduced to the cell via another suitable method. As an illustrative example, the binding agent complex 104 may be introduced to the cell via a liposome.

Per block 316, a catalyst complex 102 is introduced to the cell in accordance with the present embodiment. As disclosed herein, such a catalyst complex 102 may be cell-permeable. Alternatively, the catalyst complex 102 introduced to the cell another suitable method. As a non-limiting example, the catalyst complex 102 may be introduced via a liposome carrier.

Per block 318, a labeling agent 216 may be introduced, as in block 318. As disclosed herein, such a labeling agent 216 may be cell-permeable, though it too may be introduced to the cell via any suitable method.

The catalyst complex 102 may form a biomolecular antenna 106 with the binding agent complex 104, per block 308. For example, the ligand moiety 114 of the catalyst complex 102 may bind with the binding agent 110 of the binding agent complex104. In some embodiments, an alkyl chloride of the ligand moiety 114 may bind with a HaloTag of the binding agent 110.

After catalyst complex formation, the catalyst complex may be activated per block 310. In embodiments where the catalyst complex 102 includes a photocatalyst 112, activation may include irradiation of a wavelength of light known to activate the particular photocatalyst 112. In some embodiments, the activation can be conducted on a live cell.

Activation of the catalyst complex 102 may then activate the labeling agent 204, per block 310. In some embodiments, the photocatalyst 112 may activate the labeling agent 204 via Dexter energy transfer 212.

When activated, the labeling agent 216 may be capable of binding the label moiety 206 to a proximate biomolecule 208, per block 312. It is possible that a given activated labeling agent 216 may be quenched before it is able to bind a proximate biomolecule 208.

After labeling of a biomolecule, the label may optionally be detected, per block 314. In some embodiment where the label is detectable via, for example, fluorescence microscopy, the label may be imaged. Such imaging may indicate where the biomolecule 208 is localized, even if the interaction between the biomolecule 208 and the protein of interest 108 was relatively short and/or the biomolecule 208 and the protein of interest 108 are not co-localized at the time of imaging. In some embodiments, the label may not be fluorescent, but may be the target of a secondary label which is capable of fluorescence. In some embodiments, the label may be the target of a subsequent immunoprecipitation assay. Such an immunoprecipitation assay may be a tagged-protein immunoprecipitation or a tag-based pull-down assay. Once isolated, the labeled target protein may be subject to other orthogonal analyses. For example, an isolated labeled target protein may be quantitatively or semi-quantitatively analyzed by Western Blot or the isolated material can be quantified and identified via proteomics analysis.

In some embodiments, the process described above can be used to detect protein-protein interaction. For example, in some embodiments, the binding agent complex comprises a first protein coupled to a binding agent, where the first protein is a protein of interest for which it is desirable to probe protein-protein interaction. In some embodiments, the first protein is a ubiquitin ligase and the binding agent is a haloalkane dehalogenase, such that the binding agent complex is a fusion protein comprising the ubiquitin ligase and the haloalkane dehalogenase. The catalyst complex can then be introduced into the cell and bind to the haloalkane dehalogenase.

In some embodiments, upon activation of the catalyst complex and subsequent activation of the labeling agent, the labeling agent binds with a second protein in close proximity to the first protein. Detection of the second protein by detection of the label moiety on the labeling agent indicates a protein-protein interaction between the first and second proteins due to the fact that the second protein is only labeled if it is in close proximity to the first protein.

In some embodiments, the method of detecting protein-protein interactions may be used in the absence of any exogenous test compound in order to determine the identity of the second protein that interacts with the first protein. In some embodiments, this method may be used to identify weak protein-protein interactions, which may then serve as a therapeutic target by screening for compounds that increase the protein-protein interaction. For example, in some embodiments, the first protein is a ubiquitin ligase and the identification of weak protein-protein interaction with the ubiquitin ligase indicates that the interaction is one that could be exploited by finding compounds that enhance the interaction (for example, PROTACs or molecular glues).

In some embodiments, the method includes introducing a test compound to the cell to determine if the test compound enhances protein-protein interaction. In some such embodiments, the level of second protein labeled by the labeling agent is measured to determine the amount the protein-protein interaction is enhanced by the test compound. In other embodiments, a test compound is similarly evaluated to determine if it inhibits protein-protein interaction.

### Method of Validating Protein Associations

Just as the method disclosed herein can be used to identify a biomolecule that comes into proximity with a binding agent-tagged protein of interest, the method can be used to identify the reverse: whether the protein of interest comes into proximity with a binding agent-tagged biomolecule. Such an approach may serve to validate whether the protein of interest comes into proximity with a biomolecule. Thus, for example, the method may be used to validate whether a target disease causing protein associates with a ubiquitin ligase by forming a complex between the binding agent and the disease causing protein.

In one aspect, the present disclosure provides for a method for validating association between a disease causing protein and an ubiquitin ligase. For example, other screening methods may have suggested that an association exists and it is desirable to validate the interaction before screening for agents that would enhance the interaction, such as PROTACs or molecular glues. The method includes introducing into a cell a first binding agent complex, where the first binding agent complex includes a disease causing target protein coupled to a first binding agent, where the first binding agent is capable of binding to a first catalyst complex; introducing the first catalyst complex to the cell; forming a first biomolecular antenna comprising the first catalyst complex and the binding agent complex; introducing a first labeling agent including a first label moiety and a first reactive moiety to the cell, wherein the first reactive moiety is coupled to the first label moiety; activating the first catalyst complex, thereby activating the first labeling agent by transfer of energy from the first catalyst complex to the first reactive moiety and causing the first labeling agent to bind to the ubiquitin ligase within the cell. In some embodiments, the method further includes confirming that the ubiquitin ligase has been labeled with the labeling agent by immunoprecipitation. In some embodiments, labeled proteins from the cell lysate are first collected using functionalized beads that bind to the labeling agent (e.g., streptavidin functionalized beads if the labeling agent includes a biotin moiety). The presence of the desired ubiquitin ligase can then be determined using Western blotting with an appropriate antibody to the ligase.

### Cells and Proteins

Some embodiments include a fusion protein that comprises a protein of interest and a haloalkane dehalogenase. In some embodiments, the protein of interest is a ubiquitin ligase. Some embodiments also include a protein complex that includes the fusion protein coupled to a photocatalyst as described herein (e.g., a photocatalyst according to Formula (I)). Accordingly, some embodiments include a protein complex, including the structure:

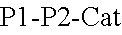

where:
P1 is a ubiquitin ligase;
P2 is a haloalkane dehalogenase;
Cat is a photocatalyst having the structure of Formula (I):

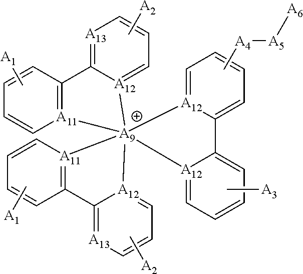

wherein:
   A1 is present 0-4 times on the ring to which it is attached and each A1 is independently selected from CH₃, CF₃, F, Cl, N(CH₃)₂, and OR₁;
   each R₁ is independently selected from H, a linear or branched alkyl group having 1-12 carbons, CHF₂, and CF₃;
   A2 is present 0-4 times on the ring to which it is attached and each A2 is independently selected from CH₃, CF₃, F, Cl, N(CH₃)₂, and OR₁;
   A3 is present 0-4 times on the ring to which it is attached and each A3 is independently selected from CH₃, CF₃, F, Cl, N(CH₃)₂, and OR₁;
   A4 is selected from null, linear or branched alkyl group having 1-12 carbons, C₆₋₁₀ aryl, C₃₋₈ cycloalkyl, 4-10 membered heterocyclyl, and 5-10 membered heteroaryl, wherein said alkyl, aryl, cycloalkyl, heterocyclyl, and heteroaryl is optionally substituted with one or more C₁₋₆ alkyl, C₁_s haloalkyl, halo, hydroxy, C₁₋₆ alkoxy, or amino;
   A5 is selected from CONH, NHCO, SONH, SO₂NH, NHSO, NHSO₂, NH, OCONH, NHCOO, C₁₋₆ alkoxy, and

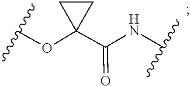 [SMILES: *NC(=O)C1(O*)CC1]

A6 is independently (PEG)a₁(CH₂)a₂Cl or (PEG)a₁(CH₂)a₂(5-6 membered heterocyclyl)(PEG)a₁(CH₂)a₂Cl, wherein each a₁ is independently an integer from 0-10 and each a₂ is independently an integer from 6-10;
A9 is a D9 metal selected from copper, vanadium, chromium, scandium, titanium, manganese, iron, cobalt, nickel, copper, zinc, yttrium, zirconium, niobium, molybdenum, technetium, ruthenium, rhodium, palladium, silver, cadmium lanthanum, hafnium, tantalum, tungsten, rhenium, osmium, iridium, platinum, gold, and mercury;
A11 and A12 are independently a C or N coordinated to A9; and
each A13 is independently CH or N.

In some embodiments, the protein complex is present within a cell. In some embodiments, the protein complex is formed by intracellular expression of the fusion protein followed by exposing the fusion protein to the photocatalyst. Accordingly, some embodiments also provide a cell that comprises a nucleotide sequence that expresses a fusion protein comprising a ubiquitin ligase and a haloalkane dehalogenase.

Some embodiments include a cell that comprises a first protein coupled to a haloalkane dehalogenase and a photocatalyst as described above (e.g., a photocatalyst according to Formula (I)). In some embodiments, the cell also includes a labeling agent as described above (e.g., a labeling agent according to Formula (III) or (III-a)).

### Terminology

As used herein, “biomolecular antenna” refers to the molecular structure formed by the union of a catalyst complex with a binding agent complex.

As used herein, “probe” or “reactive moiety” refer to a molecule capable of (1) coupling to a label, (2) being activated by a photocatalyst via Dexter energy transfer, and (3) binding to a variety of molecules when in the activated state.

As used herein, “binding agent” refers to a molecule that is (1) either capable of attaching to a protein of interest or being expressed as part of a fusion protein with the protein of interest and (2) capable of binding to the ligand moiety of a catalyst complex.

“Room temperature” is abbreviated herein as “RT.”

As used herein, “PEG” refers to the moiety having the structure:

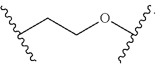 [SMILES: *CCO*]

As used herein, “alkyl” refers to a straight or branched hydrocarbon chain that is fully saturated (i.e., contains no double or triple bonds). The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 4 carbon atoms. The alkyl group may be designated as “C₁₋₄ alkyl” or similar designations. By way of example only, “C₁₋₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

As used herein, “heterocyclyl” or “heterocyclic” means a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations. In preferred six membered monocyclic heterocyclyls, the heteroatom(s) are selected from one up to three of O, N or S, and in preferred five membered monocyclic heterocyclyls, the heteroatom(s) are selected from one or two heteroatoms selected from O, N, or S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some embodiments, the heteroaryl group has 5 to 10 ring members or 5 to 7 ring members. The heteroaryl group may be designated as “5-7 membered heteroaryl,” “5-10 membered heteroaryl,” or similar designations. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinlinyl, benzimidazolyl, benzoxazolyl, benzothiazolyl, indolyl, isoindolyl, and benzothienyl.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms, although the present definition also covers the occurrence of the term “aryl” where no numerical range is designated. In some embodiments, the aryl group has 6 to 10 carbon atoms. The aryl group may be designated as “C₆₋₁₀ aryl,” “C₆ or C₁₀ aryl,” or similar designations. Examples of aryl groups include, but are not limited to, phenyl, naphthyl, azulenyl, and anthracenyl.

As used herein, “cycloalkyl” means a fully saturated carbocyclic ring or ring system. Examples include cyclopropyl, cyclobutyl, cyclopentyl, and cyclohexyl.

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. The terms “comprising,” “including,” “having,” and the like are synonymous and are used inclusively, in an open-ended fashion, and do not exclude additional elements, features, acts, operations, and so forth. That is, the above terms are to be interpreted synonymously with the phrases “having at least” or “including at least.” For example, when used in the context of a process, the term “comprising” means that the process includes at least the recited steps but may include additional steps. When used in the context of a device, the term “comprising” means that the device includes at least the recited features or components but may also include additional features or components. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately,” “about,” “generally,” and “substantially” may refer to an amount that is within less than 10% of, within less than 5% of, within less than 1% of, within less than 0.1% of, and within less than 0.01% of the stated amount.

The term “and/or” as used herein has its broadest least limiting meaning, which is the disclosure includes A alone, B alone, both A and B together, or A or B alternatively, but does not require both A and B or require one of A or one of B. As used herein, the phrase “at least one of” A, B, “and” C should be construed to mean a logical A or B or C, using a non-exclusive logical or.

Conditional language used herein, such as, among others, “can,” “could,” “might,” “may,” “e.g.,” and the like, unless specifically stated otherwise, or otherwise understood within the context as used, is generally intended to convey that certain features, elements and/or steps are optional. Thus, such conditional language is not generally intended to imply that features, elements and/or steps are in any way required or that one or more implementations necessarily include logic for deciding, with or without other input or prompting, whether these features, elements and/or steps are included or are to be always performed. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Any methods disclosed herein need not be performed in the order recited. The methods disclosed herein include certain actions taken by a practitioner; however, they can also include any third-party instruction of those actions, either expressly or by implication.

### EXAMPLES

Additional embodiments are disclosed in further detail in the following examples, which are not in any way intended to limit the scope of the claims. The following describes the cellular labeling of K-Ras interacting partners by the production of halo.KRAS in SW48 cell lines were procured from Horizon.

### Example 1. Preparation of a Labeling Partner

The description of preparation of a labeling partner is described with reference to Scheme 1:

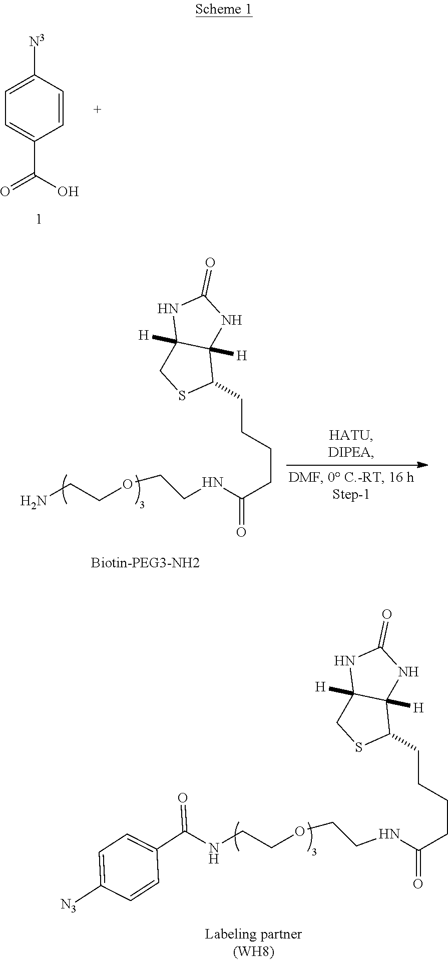

Biotin-PEG3-NH₂ (123 mg, 0.2944 mmol), HATU (280 mg, 0.7361 mmol)), and DIPEA (0.2 mL, 1.1042 mmol) were added to a stirred solution of compound 1 (60 mg, 0.368 mmol) in DMF (5 mL) at 0° C. The reaction mixture was stirred at RT for 16 hours. The progress of the reaction was monitored by thin layer chromatography (TLC) and liquid chromatography-mass spectrometry (LC-MS). After completion of the reaction, the reaction mixture was quenched with cooled water (10 mL) and extracted with ethyl acetate (2×10 mL). The combined organic layers were washed with saturated NaHCO₃ (10 mL) and brine solution (10 mL). The organic layer was dried with sodium sulphate, then filtered and concentrated under reduced pressure to yield a crude compound. The crude compound was purified by Prep-HPLC to yield 4-azido-N-(13-oxo-17-((3aS,4S,6aR)-2-oxohexahydro-1H-thieno[3,4-d]imidazol-4-yl)-3,6,9-trioxa-12-azaheptadecyl)benzamide (45 mg, yield 21%) as an off-white solid.

### Example 2. Preparation of Photocatalyst 1

The description of preparation of a photocatalyst is described with reference to Scheme 2:

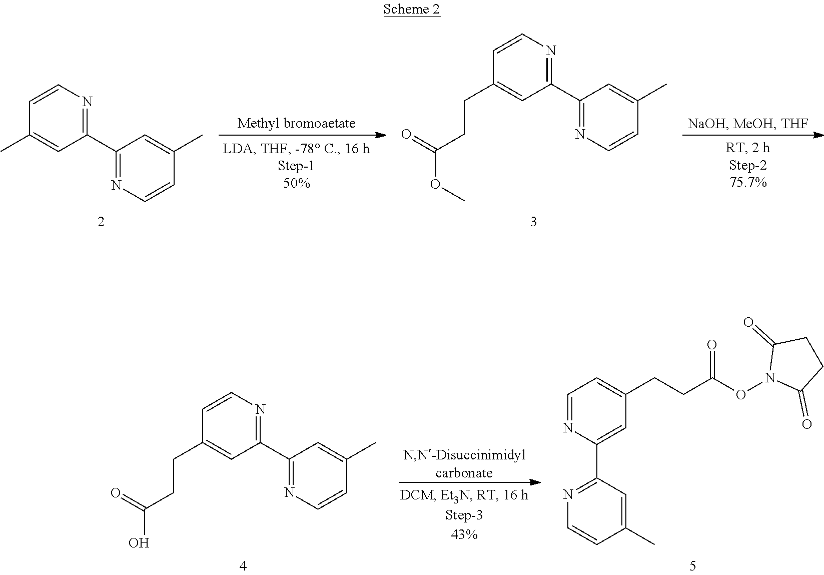

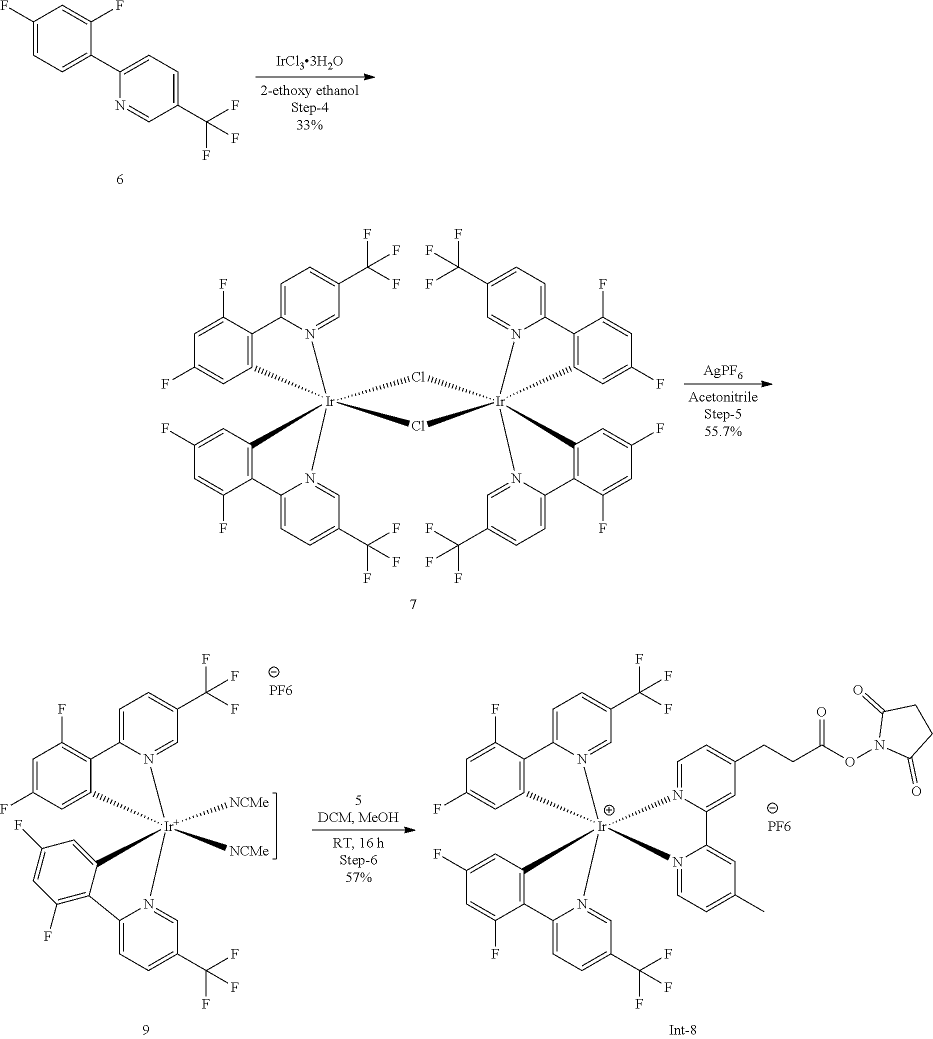

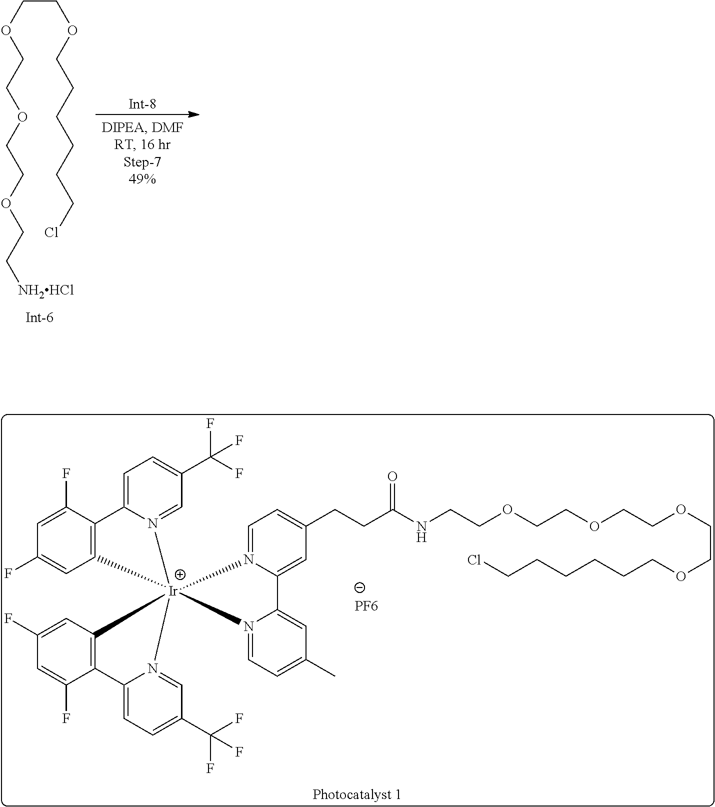

For step 1, LDA (2 M in THF, 32.56 mL, 65.13 mmol, 1.2 equivalents) was added drop wise and stirred at −78° C. for 1.5 h to a stirred solution of compound 2 (10 g, 54.277 mmol, 1.0 equivalent) in dry THE (300 mL). In another round-bottom flask (RBF), 2-Bromo methyl acetate (12.5 g, 81.415 mmol, 1.5 equivalents) was dissolved in THE (100 mL) and cooled to −78° C. The 2-Bromo methyl acetate solution was then added slowly at −78° C. and allowed to stir at RT for 16 hours. The progress of the reaction was monitored by LC-MS & TLC (30% ethyl acetate in hexane). After completion of the reaction, the reaction mixture was quenched with saturated NaHCO₃ solution (150 mL mL) and extracted with ethyl acetate (2×300 mL mL). The organic layer was dried over Na₂SO₄ and evaporated under reduced pressure to yield crude compound. The crude compound was subsequently purified by silica gel (230-400 mesh) column chromatography eluting with 0-2% methanol in DCM to yield compound 3 (7 g, yield 50%) as pale brown solid.

For step 2, 2 M NaOH (1.64 g, 40.966 mmol, 1.5 equivalents) solution at 0-10° C. was added to a stirred solution of compound 3 (7 g, 27.31 mmol, 1.0 equivalent) in a mixture of methanol (70 mL mL) and THF (21 mL mL) and stirred at RT for 2 hours. The progress of the reaction was monitored by LC-MS and TLC (10% methanol in DCM). After completion of the reaction, the reaction mixture was concentrated under reduced pressure and diluted with water (40 mL), then adjusted to a pH of 3-4 with 10% citric acid solution (30 mL). The solid was filtered, dried under vacuum, yielding compound 4 (5 g, yield 75.7%) as pale brown solid.

For step 3, trimethylamine (1.7 mL, 12.396 mmol, 1.5 equivalents) and N,N′-disuccinimidyl carbonate was added at RT to a stirred solution of compound 4 (2 g, 8.264 mmol, 1 equivalent) in dichloromethane (20 mL) and stirred at RT for 16 hours. The progress of the reaction was monitored by LC-MS and TLC (5% methanol in DCM). After completion of the reaction, the reaction mixture was diluted with water (20 mL) and extracted with dichloromethane (2×20 mL). The organic layer was dried over Na₂SO₄ and evaporated under reduced pressure to yield crude compound. The crude compound was purified by silica gel column chromatography by eluting with mobile phase 0-5% methanol in DCM, yielding compound 5 (1.2 g, yield 43%) as a pale brown liquid.

For step 4, in a clean and dry RBF, aqueous IrCl₃ (600 mg, 2.009 mmol, 1 equivalent) and compound 6 (1.145 g, 4.421 mmol, 2.2 equivalents) were added and evacuated and refilled with N₂ three times. Then a degassed solution of 2-ethoxy ethanol (24 mL) and water (8.4 mL) was added and heated to 120° C. for 16 hours. The progress of the reaction was monitored by LC-MS. After completion of the reaction, the reaction mixture was cooled to RT and yellow precipitate was filtered, washed with water (120 mL) and hexane (50 mL) and dried under vacuum to yield compound 7 (605 mg, yield 33%) as yellow solid.

For step 5, AgPF₆ (143 mg, 0.564 mmol, 2.1 equivalents) at RT was added to a stirred solution of compound 8 (400 mg, 0.268 mmol, 1 equivalent) in acetonitrile (30 mL) stirred for 16 hours. The progress of the reaction was monitored by LC-MS. After completion of the reaction, the reaction mixture was concentrated under reduced pressure to yield a crude compound. The crude compound was subsequently purified by triturating with diethyl ether (20 mL) to yield compound 9 (350 mg, yield 55.7%) as a pale-yellow solid.

For step 6, a stirred solution of compound 5 (130 mg, 0.384 mmol, 1.2 equivalents) in dichloromethane was degassed with argon for 5 minutes. Then a solution of compound 9 in dichloromethane (18 mL) was added at RT and stirred for 16 hours. The progress of the reaction was monitored by LC-MS and TLC (10% methanol in DCM). After completion of the reaction, the reaction mixture was concentrated under reduced pressure to yield crude compound. The crude compound was purified by triturating with diethyl ether (10 mL) to yield compound Int-8 (320 mg, yield 83%) as a yellow solid. mL mL mg mmol mL mL % mg

For step 7, DIPEA (0.1 mL, 0.251 mmol) and compound Int-8 (100 mg, 0.083 mmol) were added to a stirred solution of 18-chloro-3,6,9,12-tetraoxaoctadecan-1-amine hydrochloride (compound Int-6) in DMF and stirred at RT for 16 hours. Progress of the reaction was monitored by LC-MS and TLC. After completion of the reaction, the reaction mixture was diluted with water (10 mL) and extracted with EtOAc (2×10 mL). The organic layer was dried over Na₂SO₄ and evaporated under reduced pressure to yield crude compound. The crude compound was purified by flash column chromatography (FCC) using 5% MeOH in DCM, and the required fractions were concentrated under reduced pressure to yield Photocatalyst 1 (50 mg, yield 49%) as a pale-yellow solid. Photocatalyst 1 is an example photocatalyst in accordance with the present disclosure.

The description of preparation of compound Int-6 is described with reference to Scheme 3:

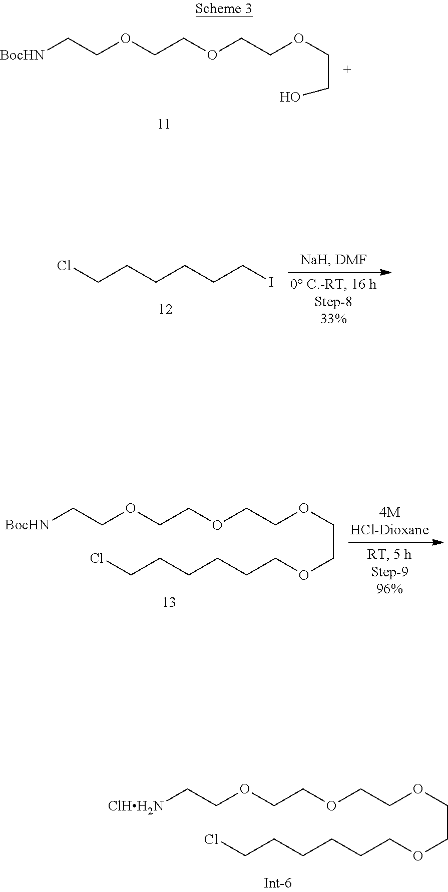

For reaction step 8, sodium hydride (1.308 g, 32.724 mmol, 1.2 equivalents) was added to a stirred solution of compound 11 (8 g, 27.27 mmol, 1 equivalent) in THF (56 mL) at 0° C. and stirred for 30 min. Then, compound 12 (8.06 g, 32.724 mmol, 1.2 equivalents) was added slowly at 0-5° C. and stirred for 16 hours at RT. The progress of the reaction was monitored by LC-MS and TLC (70% ethyl acetate in petroleum ether). After completion of the reaction, the reaction mixture was quenched with saturated ammonium chloride solution (80 mL) and extracted with ethyl acetate (2×100 mL). The organic layer was washed with water (100 mL), dried over Na₂SO₄ and evaporated under reduced pressure to yield crude compound, which was purified by silica gel column chromatography by eluting with mobile phase 0-50% ethyl acetate in petroleum ether, yielding compound 13 (3.7 g, yield 33%) as pale brown liquid.

For step 9, 4 M HCl in dioxane (19 mL) at 0-5° C. was added to a stirred solution of compound 13 (3.7 g, 12.612 mmol) in dichloromethane (37 mL) and stirred for 5 h at RT. The progress of the reaction was monitored by LC-MS and TLC (70% ethyl acetate in petroleum ether). After completion of the reaction, the reaction mixture was concentrated under reduced pressure to yield compound Int-6 (3 g, yield 96%), which was used in step 7 of scheme 2 without further purification.

### Example 3. Preparation of a Photocatalyst 1D

The description of preparation of a photocatalyst is described with reference to Scheme 4:

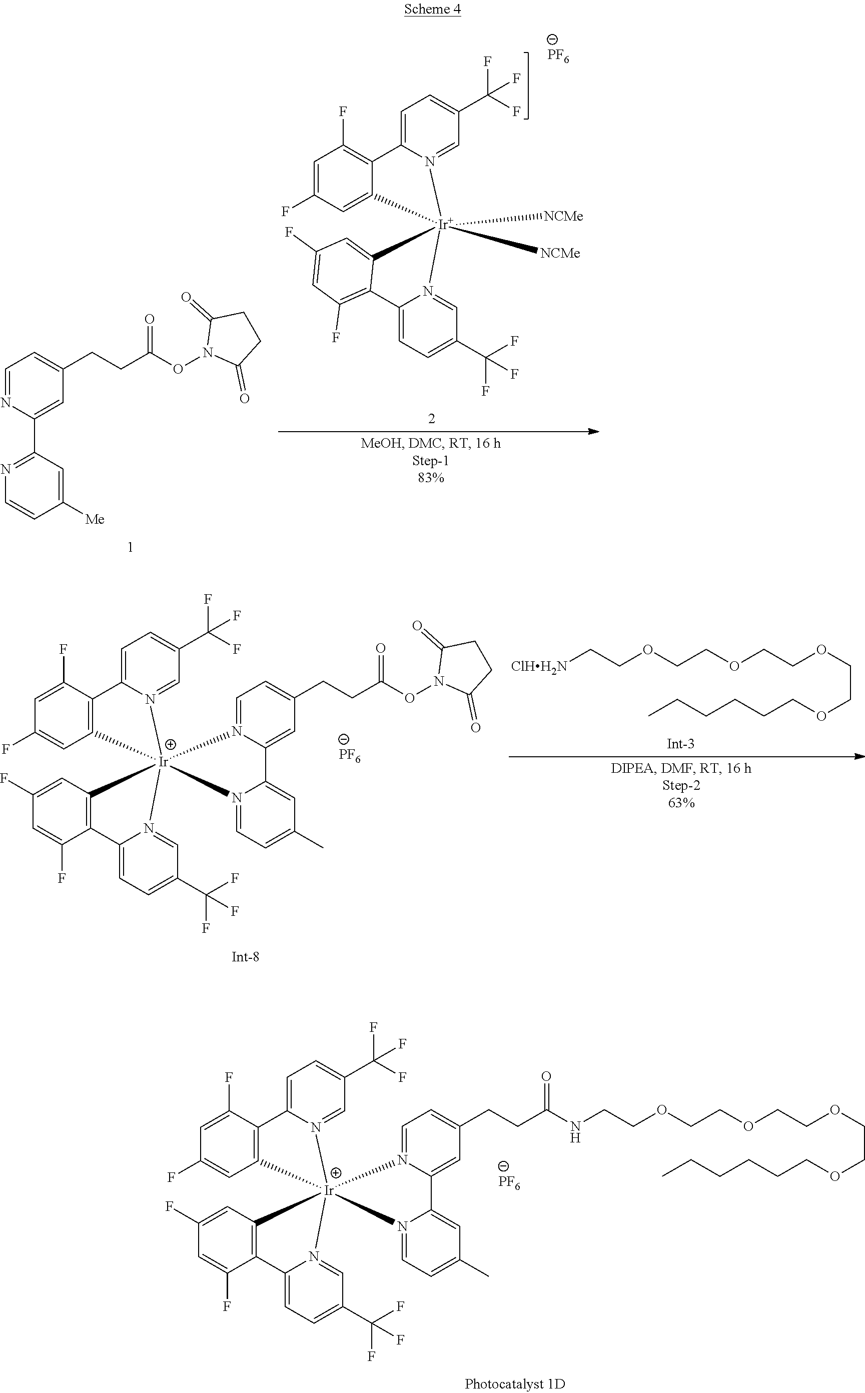

For step 1, a stirred solution of compound 1 (130 mg, 0.384 mmol, 1.2 equivalents) in dichloromethane (45 mL) was degassed with argon for 5 minutes. Then, a solution of compound 2 in dichloromethane (18 mL) was added at RT and stirred for 16 hours. The progress of the reaction was monitored by LC-MS and TLC (10% methanol in DCM). After completion of the reaction, the reaction mixture was concentrated under reduced pressure to yield crude compound, which was purified by triturating with diethyl ether (10 mL), to further yield compound Int-8 (320 mg, yield 83%) as a yellow solid.

For step 2, DIPEA (0.3 mL, 0.159 mmol, 2 equivalents) at RT was added to a solution of compound Int-3 (25 mg, 0.0796 mmol, 1 equivalent) in DMF (1.5 mL) and stirred for 10 minutes. Then compound Int-8 (95 mg, 0.0796 mmol, 1 equivalent) was added and stirred at RT for 16 hours. The progress of the reaction was monitored by LC-MS and TLC (10% methanol in DCM). After completion of the reaction, the reaction mixture was concentrated under reduced pressure to yield crude compound, which was purified by silica gel (230-400 mesh) column chromatography by eluting with mobile phase 0-5% methanol in DCM, to yield Photocatalyst 1D (1.25 g, yield 63%) as a yellow solid. Photocatalyst 1D is an example photocatalyst in accordance with the present disclosure.

The description of preparation of compound Int-3 is described with reference to Scheme 5:

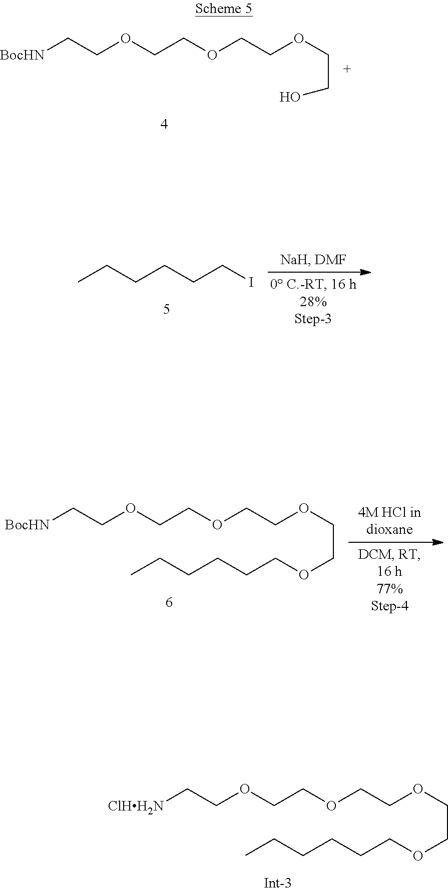

Sodium hydride (302 mg, 8.181 mmol, 1.2 equivalents) at 0° C. was added to a stirred solution of compound 4 (2 g, 6.817 mmol, 1 equivalent) in a mixture of DMF (10 mL) and THF (10 mL), and was stirred for 30 min. at RT. Then compound 5 (1.73 g, 8.18 mmol, 1.2 equivalents) was added slowly at 0-5° C. and stirred at RT for 16 hours. The progress of the reaction was monitored by LC-MS and TLC (70% ethyl acetate in petroleum ether). After completion of the reaction, the reaction mixture was quenched with saturated ammonium chloride solution (20 mL) and extracted with ethyl acetate (2×20 mL). The organic layer was washed with water (30 mL), dried over Na₂SO₄, and evaporated under reduced pressure to yield crude compound, which was purified by silica gel column chromatography by eluting with mobile phase 0-50% ethyl acetate in petroleum ether, further yielding compound 6 (700 mg, yield 28%) as pale-yellow gummy liquid.

### Example 4. Preparation of Photocatalysts 2, 3, and 4

The description of preparation of a photocatalyst is described with reference to Scheme 6:

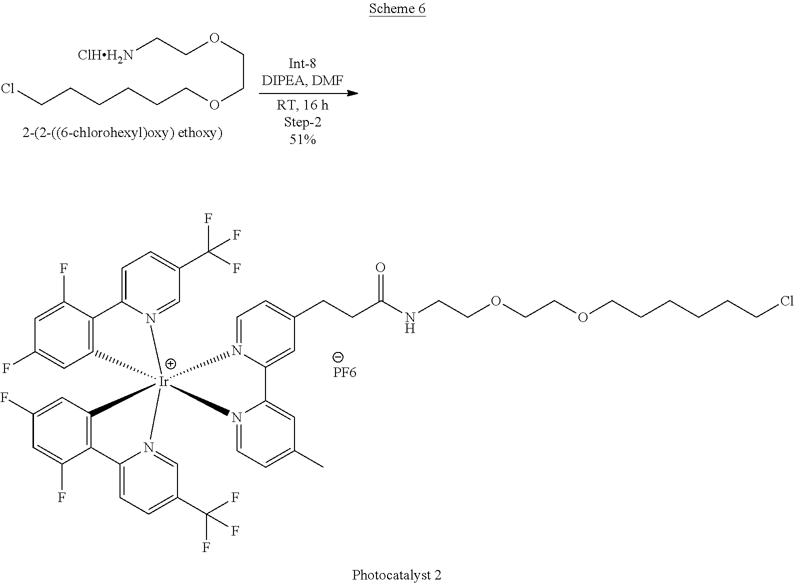

In accordance with Scheme 6, DIPEA (0.1 mL, 0.251 mmol) and compound Int-8 (with reference to Example 2) (100 mg, 0.083 mmol) were added to a stirred solution of 2-(2-((6-chlorohexyl)oxy) ethoxy) ethan-1-amine hydrochloride (20 mg, 0.083 mmol) in DMF (10 mL) and stirred at RT for 16 hours. The progress of the reaction was monitored by TLC and LC-MS. After completion of the reaction, the reaction mixture was diluted with water (10 mL) and extracted with EtOAc (2×10 mL). The organic layer was dried over Na₂SO₄ and evaporated under reduced pressure to yield crude compound. The crude compound was purified by FCC using 5% MeOH in DCM, the required fractions were concentrated under reduced pressure to yield Photocatalyst 2 (50 mg, yield 51%) as a pale-yellow solid. Photocatalyst 2 is an example photocatalyst in accordance with the present disclosure.

The description of preparation of a photocatalyst is described with reference to Scheme 7:

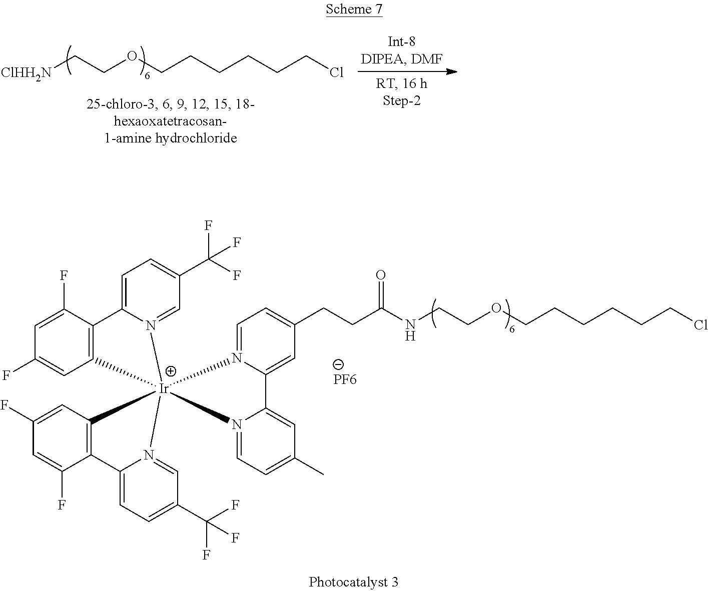

In accordance with Scheme 7, DIPEA (0.1 mL, 0.251 mmol) and compound Int-8 (with reference to Example 2) (100 mg, 0.083 mmol) were added to a stirred solution of 24-chloro-3,6,9,12,15,18-hexaoxatetracosan-1-amine hydrochloride (20 mg, 1.30 mmol) in DMF (10 mL) and stirred at RT for 16 hours. The progress of the reaction was monitored by TLC and LC-MS. After completion of the reaction, the reaction mixture was diluted with water (10 mL) and extracted with EtOAc (2×10 mL). The organic layer was dried over Na₂SO₄ and evaporated under reduced pressure to yield crude compound. The crude compound was purified by FCC using 4-5% MeOH in DCM, the required fractions were concentrated under reduced pressure to yield Photocatalyst 3 (38 mg, yield 40%) as an off-white solid. Photocatalyst 3 is an example photocatalyst in accordance with the present disclosure.

The description of preparation of a photocatalyst is described with reference to Scheme 8:

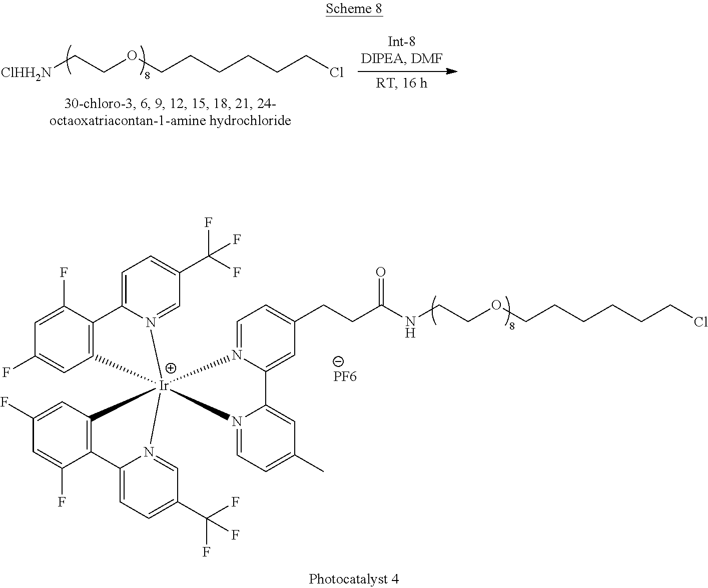

In accordance with scheme 8. DIPEA (0.1 mL, 0.251 mmol) and compound Int-8 (with reference to Example 2) (100 mg, 0.083 mmol) was added to a stirred solution of 30-chloro-3,6,9,12,15,18,21,24-octaoxatriacontan-1-amine hydrochloride (100 mg, 0.087 mmol) in DMF (10 mL) and was stirred at RT for 16 hours. The progress of the reaction was monitored by TLC and LC-MS. After completion of the reaction, the reaction mixture was diluted with water (10 mL) and extracted with EtOAc (2×10 mL). The organic layer was dried over Na₂SO₄ and evaporated under reduced pressure to yield crude compound. The crude compound was purified by FCC using 5% MeOH in DCM, and the required fractions were concentrated under reduced pressure to yield Photocatalyst 4 (29 mg, yield 40%) as an off-white solid. Photocatalyst 4 is an example photocatalyst in accordance with the present disclosure.

### Example 5. Preparation of Photocatalyst 5

The description of preparation of a photocatalyst is described with reference to Scheme 9:

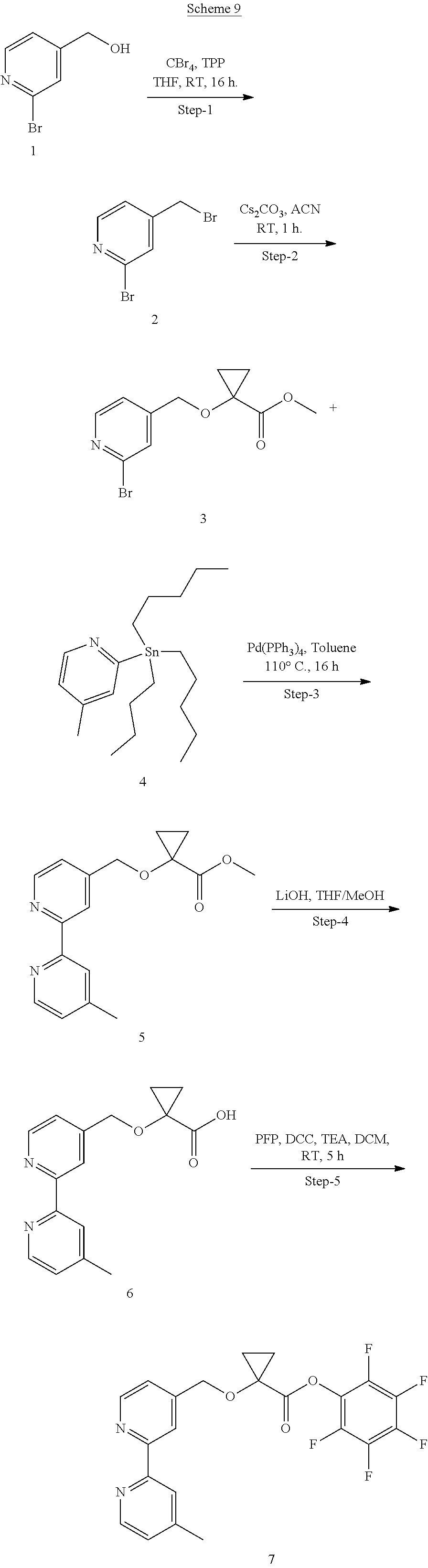

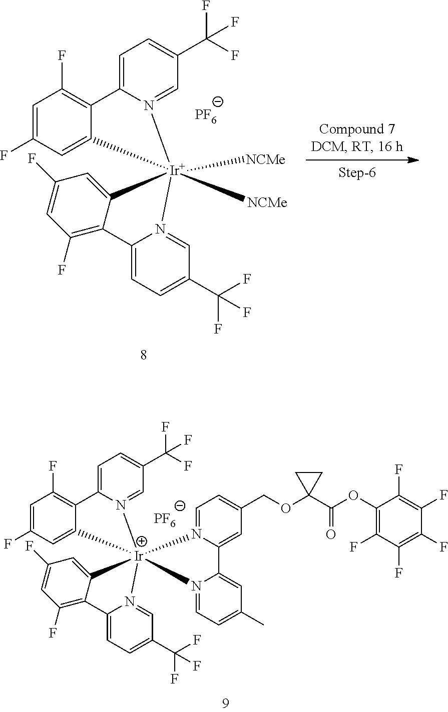

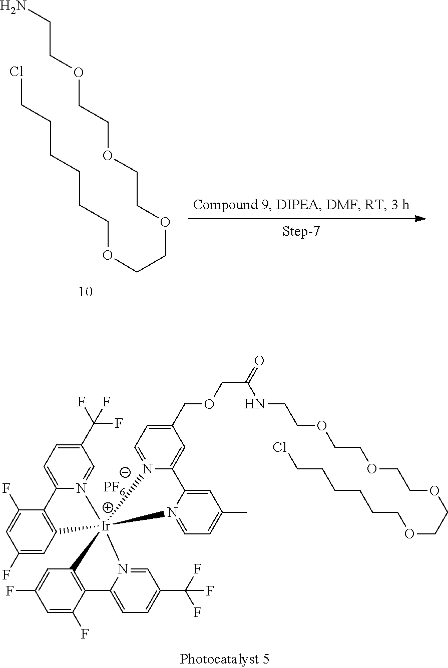

With reference to Scheme 9, step 1, CBr₄ (6.349 g, 19.146 mmol, 1.2 equivalents) was added to a stirred solution of (2-bromopyridin-4-yl)methanol 1 (3.0 g, 15.95 mmol, 1.0 equivalent), triphenylphosphine (5.022 g, 19.14 mmol, 1.2 equivalents) in THF (30 mL) and stirred for 16 hours at RT. After completion of the reaction, the solution was diluted with ice cold H₂O and extracted with ethyl acetate (3×100 mL), the organic layer washed with brine (50 mL) and dried over anhydrous sodium sulphate, concentrated under reduced pressure to yield crude compound. The crude compound was purified by column chromatography using PE and ethyl acetate as eluent. The required fractions were concentrated under reduced pressure to yield 2-bromo-4-(bromomethyl) pyridine 2 (3.2 g, yield 79.93%) as a white solid.

For step 2, to a stirred solution of 2-bromo-4-(bromomethyl) pyridine 2 (2 g, 7.971 mmol, 1.0 equivalents), methyl 1-hydroxycyclopropane-1-carboxylate 3 (

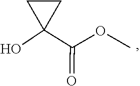

1.111 g, 9.565 mmol, 1.2 equivalents) in ACN (40.000 mL) and Cesium carbonate (7.791 g, 23.912 mmol, 3.0 equivalents) were added at RT and stirred for 16 hours. After completion of starting material, the reaction mixture was filtered and dried under reduced pressure to yield crude compound, which was purified by column chromatography using petroleum ether and ethyl acetate as eluent. The required fractions were concentrated under reduced pressure to yield methyl 1-((2-bromopyridin-4-yl)methoxy)cyclopropane-1-carboxylate 3 (2.1 g, yield 92%) as a white solid.

For step 3, 4-methyl-2-(tributylstannyl)pyridine (0.160 g, 0.41 mmol, 1.2 equivalents) in toluene (1.0 mL) was added to a stirred solution of methyl 1-((2-bromopyridin-4-yl)methoxy)cyclopropane-1-carboxylate (0.1 g, 0.34 mmol, 1.0 equivalents). The mixture was degassed with N₂ for 15 minutes. Pd(PPh₃)₄(0.081 g, 0.07 mmol, 0.2 equivalents) was added, and the mixture was heated to 110° C. for 16 hours. The progress of the reaction was monitored by LC-MS. After complete consumption of starting material, the reaction mixture was diluted with petroleum ether (150 mL), filtered, and dried under reduced pressure to yield 6-bromo-2,3-dihydro-[1,4]dioxino[2,3-f]75uinoline-10-ol 5 (3 g, yield 81%) as an off-white solid.

For step 4, aqueous LiOH solution was added at RT to a stirred solution of methyl 1-((4′-methyl-[2,2′-bipyridin]-4-yl)methoxy)cyclopropane-1-carboxylate (0.1 g, 0.349 mmol, 1.0 equivalents) in MeOH and THF. The mixture was stirred for 3 hours. The progress of the reaction was monitored by LC-MS. After complete consumption of starting material, the reaction mixture was concentrated to yield crude compound, then dissolved in water, acidified with KHSO₄ Solution (pH ˜2) and the extracted with EtOAc (3×30 mL). The organic layer was dried over Na₂SO₄ and concentrated under reduced pressure to yield 1-((4′-methyl-[2,2′-bipyridin]-4-yl)methoxy)cyclopropane-1-carboxylic acid 6 (3 g, yield 81%) as an off-white solid.

For step 5, DCC (1.6 g, 7.19 mmol, 1.1 equivalents) and 2,3,4,5,6-pentafluorophenol (1.4 g, 7.09 mmol, 1.1 equivalents) was added to a stirred solution of 1-((4′-methyl-[2,2′-bipyridin]-4-yl)methoxy)cyclopropane-1-carboxylic acid 7 (2 g, 7.04 mmol, 1 equivalent) in 1,4 Dioxane (20 mL) at RT and stirred for 5 hours. The progress of the reaction was monitored by LC-MS and TLC (40% ethyl acetate in petroleum ether). After completion of the reaction, the reaction mixture was diluted with water (50 mL) and extracted with diethyl ether (3×50 mL). The organic layer was dried over Na₂SO₄ and evaporated under reduced pressure to yield crude compound, which was purified by silica gel column chromatography by eluting with mobile phase 10-15% ethyl acetate in petroleum ether, The required fractions were concentrated under reduced pressure to yield Compound 7 (1.3 g, yield 70%) as an off-white solid.

For step 6, compound 8 (0.3 g, 0.44 mmol, 1 equivalent) was added to a solution of Compound 7 (0.2 g, 0.44 mmol, 1 equivalent) in DCM (5 mL) at RT and stirred for 16 hours. Progress of the reaction was monitored by LC-MS and TLC (40% ethyl acetate in petroleum ether). After completion of the reaction, the reaction mixture was concentrated under reduced pressure to yield crude compound. The crude product was stirred in pentane (10 mL) and filtered to obtain compound 9 (1.25 g, yield 63%) as a yellow solid.

For step 7, compound 10 (60 g, 0.194 mmol, 1.5 equivalents) and DIPEA (0.06 mL, 0.388 mmol, 3 equivalents) were added to a solution of compound 9 (0.15 g, 0.129 mmol, 1 equivalent) in DMF (2 mL) at RT, stirred for 16 hours. Progress of the reaction was monitored by LC-MS and TLC (40% Ethyl acetate in petroleum ether). After completion of the reaction, the reaction mixture was concentrated under reduced pressure to yield crude compound, which was purified by silica gel column chromatography (230-400 mesh) by eluting with mobile phase 0-5% methanol in DCM. The required fractions were concentrated under reduced pressure to yield Photocatalyst 5 (0.05 g, yield 30%) as a yellow solid. Photocatalyst 5 is an example photocatalyst in accordance with the present disclosure.

### Example 6. Preparation of Photocatalysts 6

The description of preparation of a photocatalyst is described with reference to Scheme 10:

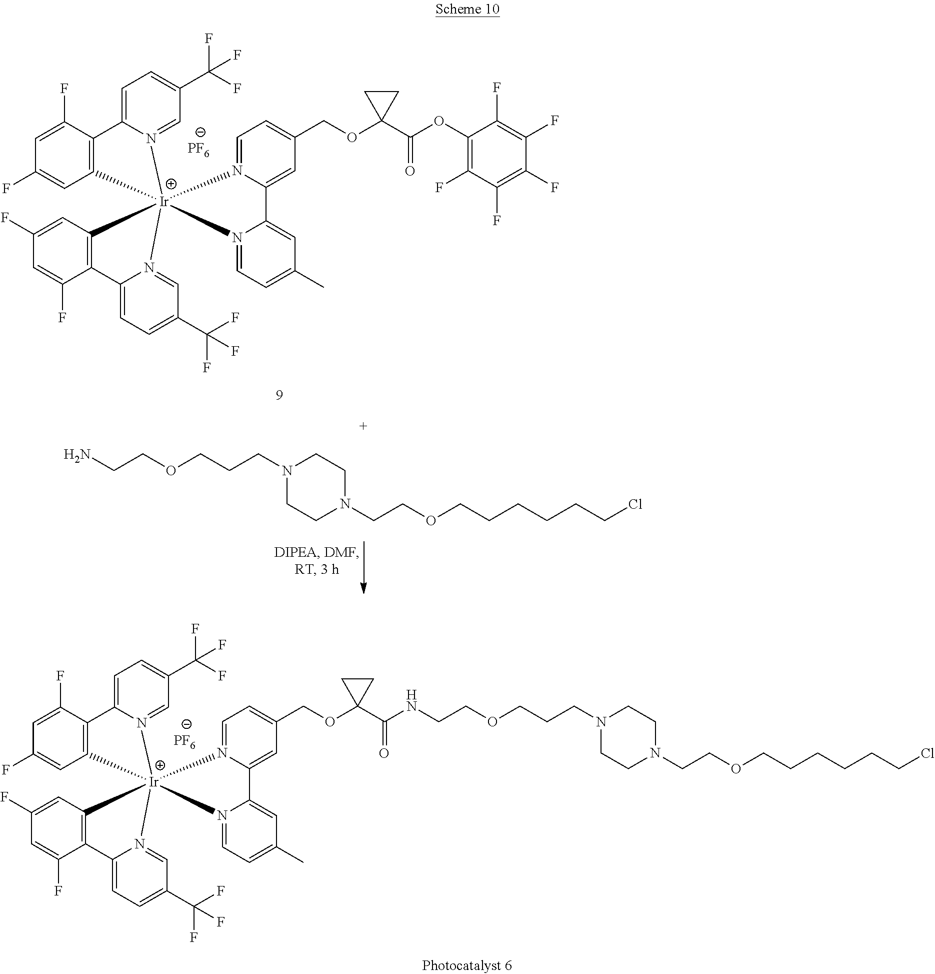

In accordance with Scheme 10, compound 9 (with reference to Example 5) (0.124 g, 0.345 mmol, 2 equivalents) and DIPEA (0.04 mL, 0.521 mmol, 3 equivalents) were added to a solution of 2-(3-(4-(2-((6-chlorohexyl)oxy)ethyl)piperazin-1-yl)propoxy)ethan-1-amine (0.2 g, 0.179 mmol, 1 equivalent) in DMF (2 mL) at RT. The resulted reaction mixture was stirred for 16 hours at RT. The progress of the reaction was monitored by LC-MS and TLC (10% MeOH in DCM). After completion of the reaction, the reaction mixture was concentrated under reduced pressure to yield crude compound, which was purified by silica gel (NH Silica gel mesh) column chromatography by eluting with mobile phase 0-5% methanol in DCM. The compound fractions were collected and concentrated under reduced pressure to yield Photocatalyst 6 (0.083 g, yield 47%). A 28 mg (90% LC-MS) lot of Photocatalyst 6 was purified by Prep HPLC to yield pure Photocatalyst 6 (19.3 mg, 97% LC-MS). Photocatalyst 6 is an example photocatalyst in accordance with the present disclosure.

### Example 7. Preparation of Photocatalyst 7

The description of preparation of a photocatalyst is described with reference to Scheme 11:

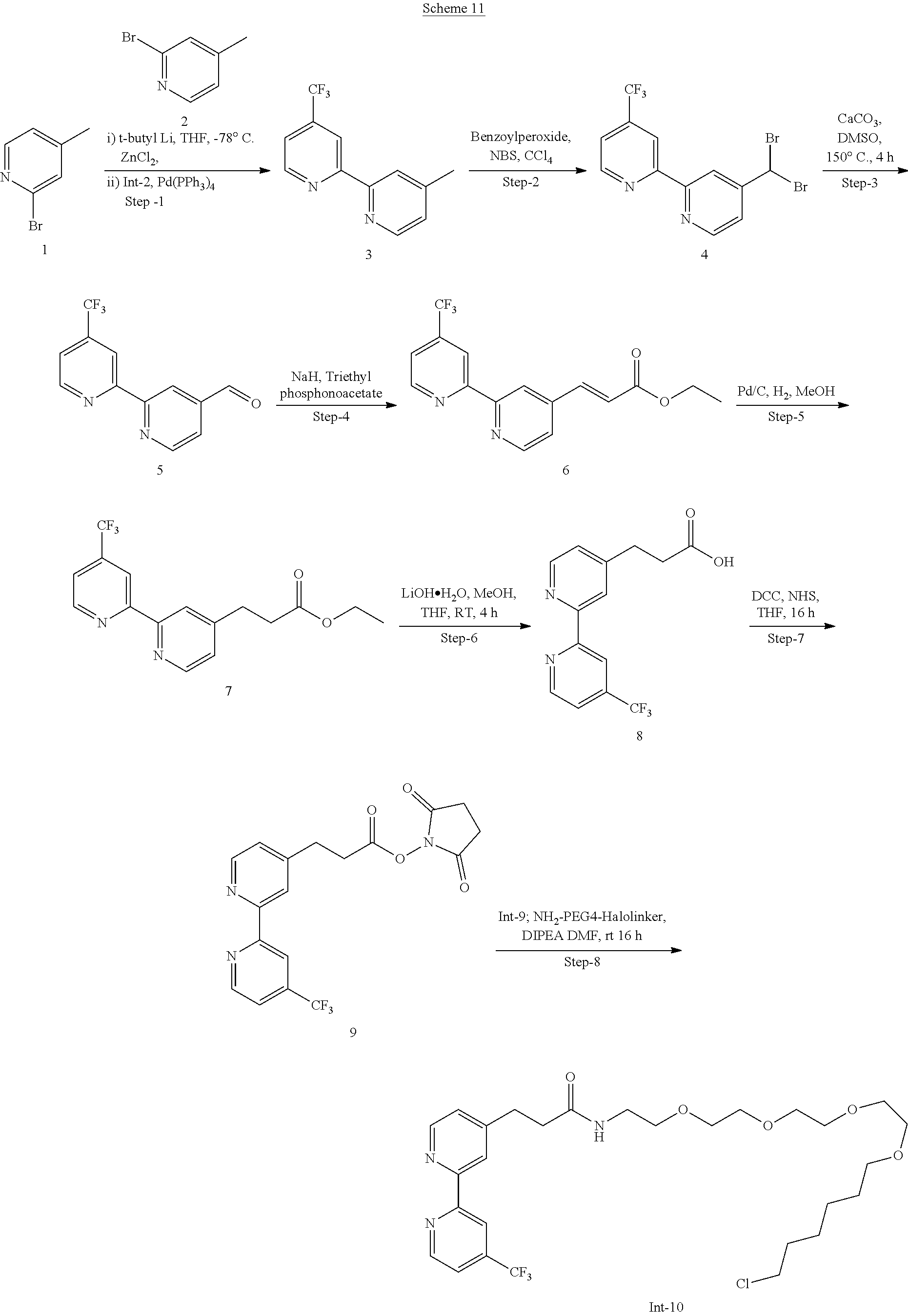

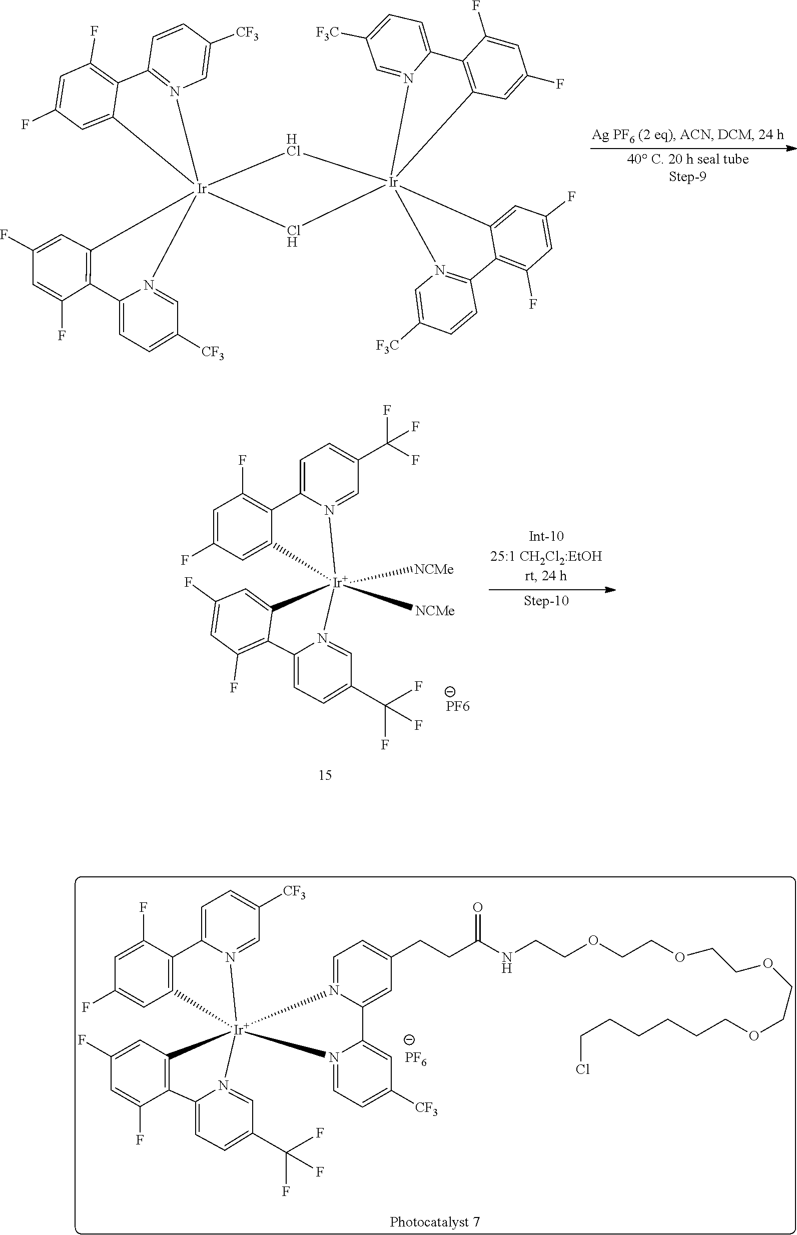

For step 1 of Scheme 11, a solution of compound 1 (6 g, 0.0351 mmol) in THF was added dropwise at −78° C. to a stirred solution of t-butyl lithium (70 mL, 0.0701 mmol) in THF and the reaction mixture was stirred for 30 minutes. ZnCl₂ solution (11.9 g, 0.088 mmol) in THF (40 mL) was added and the reaction mixture was stirred at RT for 2 hours. A pre-prepared solution of compound 2 (8 g, 0.0315 mmol) and tetrakis triphenylphosphine in THF was added dropwise at RT. The resulting reaction mixture was refluxed for 36 hours. The reaction was monitored by TLC and LC-MS. The reaction mixture was cooled to RT and quenched with saturated solution of EDTA and the pH=8 was adjusted with NaHCO₃. The aqueous layer was extracted with EtOAc. The organic layer was dried over Na₂SO₄, filtered, and concentrated under reduced pressure. The crude reaction mixture was purified by column chromatography to yield compound 3 as an off-white solid (4.2 g, yield 50%).

For step 2, benzoyl peroxide (0.0021 mmol) and then NBS (0.021 mmol) were added to a stirred solution of compound 3 (1 g, 0.0042 mmol) in CCl₄ and the resulting reaction mixture was refluxed for 16 hours. The reaction was monitored by TLC. The reaction mixture was cooled to RT, quenched with water, extracted with DCM. The organic layer was dried over Na₂SO₄ filtered and concentrated under reduced pressure to yield crude compound 4. The obtained crude compound 4 was used in next step without further purification.

For step 3, CaCO₃ (0.00261 mmol) was added to a stirred solution of compound 4 (crude 2.6 g, 0.0066 mmol) in DMSO and the resulting reaction mixture was stirred at 150° C. for 16 hours. The reaction was monitored by TLC and LC-MS. The reaction mixture was cooled to RT, quenched with water, and extracted with EtOAc. The organic layer was dried over Na₂SO₄, filtered and concentrated under reduced pressure. The crude reaction mixture was purified by column chromatography to yield compound 5 as a white solid (200 mg, yield 38%).

For step 4, NaH (47.6 mg 1.98 mmol) was added portionwise to a stirred solution of triethyl phosphonate acetate (449.8 mg, 1.98 mmol) in THE was added at 0° C. and the reaction mixture was stirred for 30 min. The solution of compound 5 (200 mg, 0.793 mmol) in THF was added and the reaction mixture was stirred at RT for 3 hours. The reaction was monitored by TLC. The reaction mixture was quenched with saturated solution of NH₄Cl at 0° C. The aqueous layer was extracted with EtOAc. The organic layer was dried over Na₂SO₄, filtered and concentrated under reduced pressure. The crude reaction mixture was purified by column chromatography to yield compound 6 as a white solid (192 mg, yield 74%).

For step 5, palladium on carbon (5% mmol) was added to a stirred solution of compound 6 (120 mg, 0.372 mmol) in methanol at RT. The resulting suspension was stirred under hydrogen atmosphere for 4 hours. The reaction was monitored by TLC. The reaction mixture was filtered through celite bed and filtrate was concentrated under reduced pressure to yield crude compound 7. The obtained crude compound 7 was used in next step without further purification.

For step 6, aqueous LiOH (50 mg, 1.23 mmol) was added to a stirred solution of compound 7 (crude 200 mg, 0.6172 mmol) in a mixture of methanol and THF (1:1) at RT and the resulting reaction mixture was stirred for 4 hours. The reaction was monitored by TLC. The reaction mixture was quenched with water. The aqueous layer was acidified with citric acid and extracted with DCM. The organic layer was dried over Na₂SO₄, filtered and concentrated under reduced pressure. The crude reaction mixture was purified by column chromatography to yield compound 8 as an off-white solid (144 mg, yield 79%).

For step 7, DCC (38 mg, 0.185 mmol), followed by N-Hydroxysuccinimide (23.3 mg, 0.2026 mmol), was added to a stirred solution of compound 8 (50 mg, 0.168 mmol) in THF at RT. The resulting reaction mixture was stirred for 16 hours. The reaction was monitored by TLC and LC-MS. The reaction mixture was quenched with water. The aqueous layer was extracted with EtOAc. The organic layer was dried over Na₂SO₄, filtered, and concentrated under reduced pressure. The crude reaction mixture was purified by reverse phase column chromatography to yield compound 9 as a white solid (20 mg, yield 30%).

For step 8, DIPEA (59.2 mg, 0.458 mmol), followed by NH₂-PEG₄-Halolinker (referred to as compound Int-6 in Example 2) (45 mg, 0.127 mmol), was added to a stirred solution of compound 9 (50 mg, 0.127 mmol) in DMF at RT. The resulting reaction mixture was stirred for 16 hours. The reaction was monitored by TLC and LC-MS. The reaction mixture was quenched with water. The aqueous layer was extracted with EtOAc. The organic layer was dried over Na₂SO₄, filtered and concentrated under reduced pressure. The crude reaction mixture was purified by reverse phase column chromatography to yield compound Int-10 as a pale brown solid (23 mg, yield 30%).

For step 9, dry ACN (0.8 mL), followed by AgPF₆(34.7 mg, 0.1377 mmol), was added to a stirred solution of compound 14 (100 mg, 0.0672 mmol) in dry DCM (4 mL) in a glove box. The resulting reaction mixture was stirred at 40° C. for 20 hours in a seal tube. The reaction was monitored by LC-MS. The reaction mixture was cooled to RT and concentrated under reduced pressure. The crude reaction mixture was dissolved in acetone, filtered, and filtrate was concentrated under reduced pressure to yield compound 15 as a yellow solid (90 mg, yield 72%).

For step 10, compound Tnt-10 (20 mg, 0.0346 mmol) was added to a stirred solution of compound 15 (27 mg, 0.0289 mmol) in mixture of solvent DCM (0.8 mL) and EtOH (0.02 mL) (25:1). The resulting reaction mixture was stirred at RT for 20 hours. The reaction was monitored by LC-MS. The reaction mixture was concentrated under reduced pressure. The crude reaction mixture was diluted with acetone, the mixture was filtered, and the filtrate was concentrated under reduced pressure. The crude compound was triturated with pentane to yield Photocatalyst 7 as a yellow solid (25 mg, yield 52%). Photocatalyst 7 is an example photocatalyst in accordance with the present disclosure.

### Example 8. Preparation of Photocatalyst 8

The description of preparation of a photocatalyst is described with reference to Scheme 12:

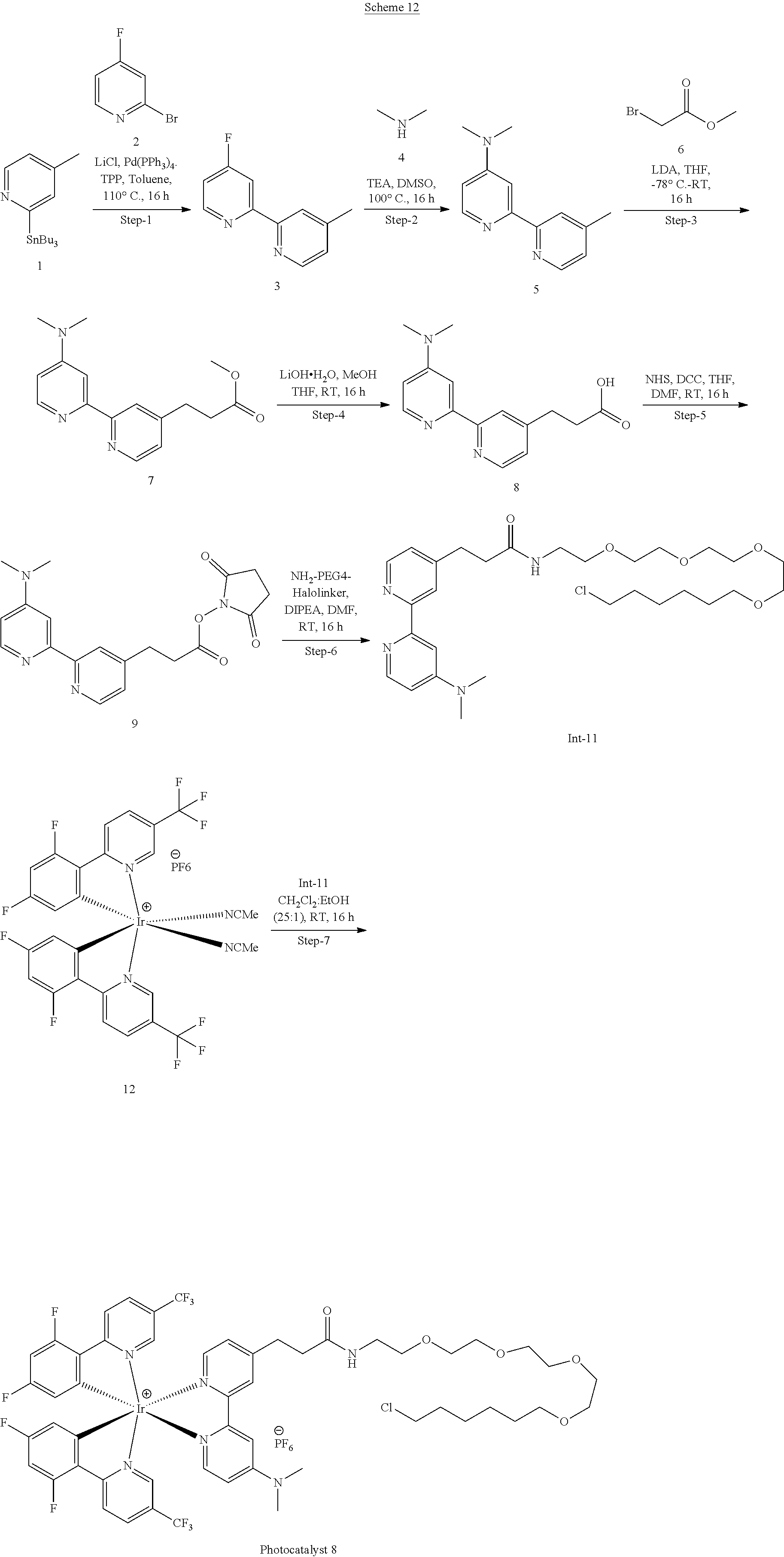

With reference to Scheme 12, step 1, a stirred solution of 4-methyl-2-(tributylstannyl)pyridine 1 (2.0 g, 5.23 mmol) and 2-bromo-4-fluoropyridine (1.38 g, 7.85 mmol) in Toluene (40 mL) was degassed with argon for 10 min. Then TPP (137 mg, 0.52 mmol), LiCl (0.66 g, 15.7 mmol), and Pd(PPh₃)₄(604 mg, 0.52 mmol) were added and the reaction mixture was stirred at 110° C. for 16 hours. The reaction was monitored by TLC and LC-MS. The reaction mixture was cooled to RT, quenched with water and extracted with EtOAc. The organic layer was dried over Na₂SO₄, filtered, and concentrated under reduced pressure. The crude reaction mixture was purified by column chromatography to yield compound 3 as an off white semi-solid (0.5 g, yield 51%).

For step 2, dimethylamine (2 M in MeOH, 2.66 mL, 5.31 mmol), followed by triethylamine (1.12 mL, 7.9 mmol), was added to a stirred solution of compound 3 (0.5 g, 2.65 mmol) in DMSO (10 mL) and the resulting reaction mixture was heated at 100° C. for 16 hours. The reaction was monitored by TLC. The reaction mixture was cooled to RT and concentrated under reduced pressure to yield crude compound. The obtained crude compound was purified by reverse phase column chromatography to yield compound 5 as a white solid (270 mg, yield 48%).

For step 3, LDA (2 M in THF, 1.26 mL, 2.53 mmol) was added to a stirred solution of compound 5 (270 mg, 1.26 mmol) in THF (6 mL) at −78° C. The reaction mixture was stirred at −78° C. for 45 minutes. Methyl 2-bromoacetate (380 mg, 2.53 mmol) at −78° C. was added and the reaction mixture was stirred at RT for 16 hours. The reaction was monitored by TLC and LC-MS. The reaction mixture was quenched with aqueous NH₄Cl solution and extracted with EtOAc. The organic layer was dried over Na₂SO₄, filtered, and concentrated under reduced pressure to yield crude compound. The obtained crude compound was purified by reverse-phase column chromatography to yield compound 7 as a light brown liquid (82 mg, yield 22%).

For step 4, aqueous LiOH (13.4 mg, 0.56 mmol) was added to a stirred solution of compound 7 (80 mg, 0.28 mmol) in methanol (0.4 mL) and THF (0.4 mL) at RT. The resulting reaction mixture was stirred for 16 hours. The reaction was monitored by TLC. Volatiles were evaporated under reduced pressure to yield crude residue. The crude residue was purified by reverse phase column chromatography to yield compound 8 as a light brown liquid (52 mg, yield 68%).

For step 5, DCC (76 mg, 0.36 mmol) followed by N-Hydroxysuccinimide (42 mg, 0.36 mmol) was added to a stirred solution of compound 8 (50 mg, 0.18 mmol) in THF:DMF (2:1, 1.5 mL) at RT. The resulting reaction mixture was stirred at RT for 16 hours. The reaction was monitored by TLC and LC-MS. The reaction mixture was quenched with water and extracted with EtOAc. The organic layer was dried over Na₂SO₄, filtered, and concentrated under reduced pressure to yield compound 9 as a white semi-solid (62 mg, yield 91%).

For step 6, DIPEA (80 μL, 0.48 mmol), followed by NH₂-PEG4-Halolinker (50 mg, 0.16 mmol), was added to a stirred solution of compound 9 (60 mg, 0.16 mmol) in DMF (1 mL) at RT. The resulting reaction mixture was stirred for 16 hours. The reaction was monitored by TLC and LC-MS. The reaction mixture was purified by reverse phase column chromatography to yield compound Int-11 as a pale brown, sticky liquid (21 mg, yield 23%).

For step 7, compound 12 (33 mg, 0.035 mmol) was added to a stirred solution of compound Int-11 (20 mg, 0.035 mmol) in DCM (0.8 mL) and EtOH (20 μL) at RT. The resulting reaction mixture was stirred at RT for 16 hours. The reaction was monitored by LC-MS. The reaction mixture was concentrated under reduced pressure. The crude reaction mixture was diluted with acetone, the mixture was filtered, and the filtrate was concentrated under reduced pressure. The crude compound was purified by reverse phase column chromatography to yield Photocatalyst 8 as a yellow solid (7.7 mg, yield 17%). Photocatalyst 8 is an example photocatalyst in accordance with the present disclosure.

### Example 9. Preparation of Photocatalyst 9

The description of preparation of a photocatalyst is described with reference to Scheme 13:

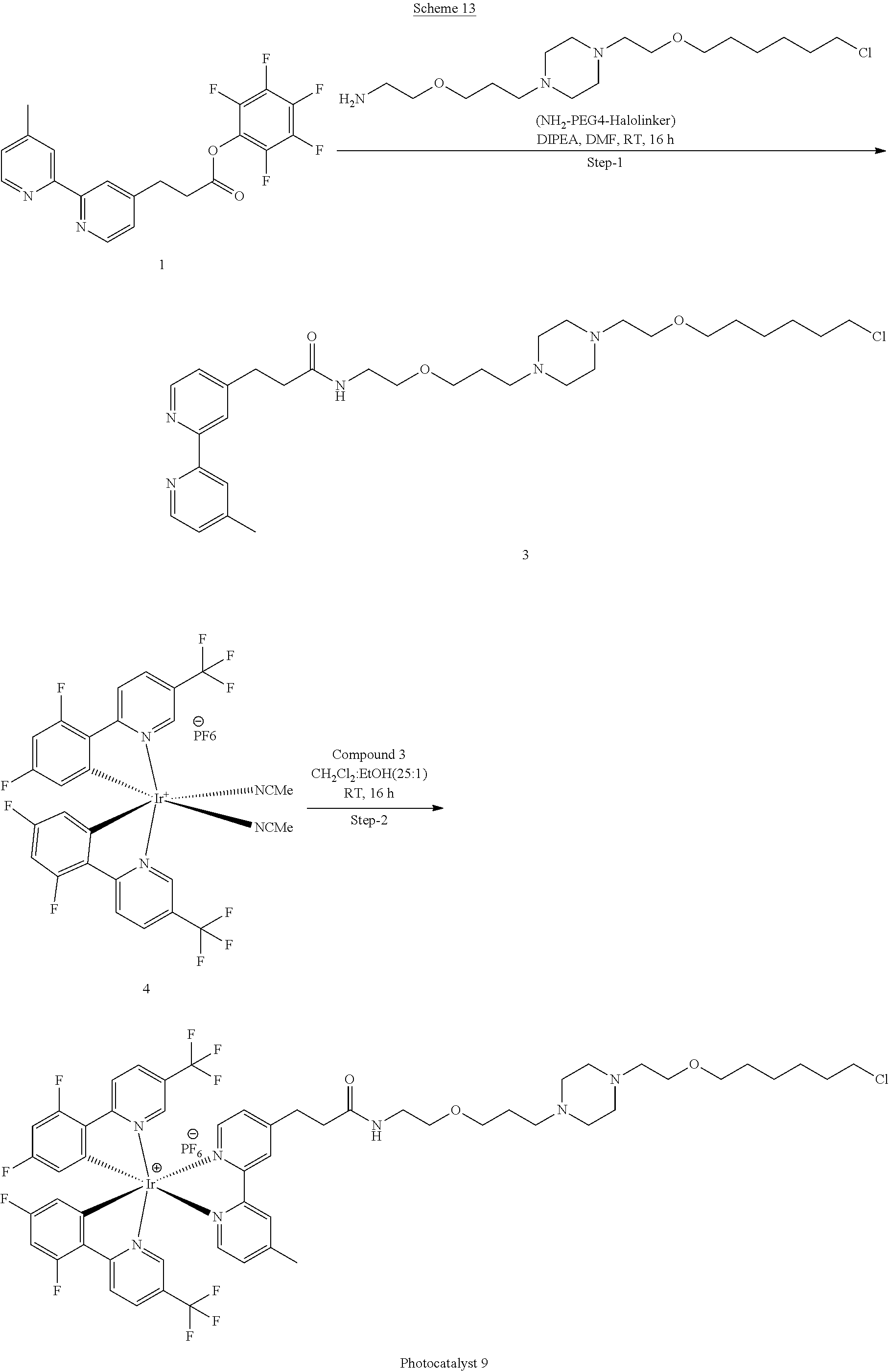

With reference to Scheme 13, step 1, DIPEA (0.047 mL, 0.36 mmol) followed by NH₂-PEG4-Halolinker (47 mg, 0.13 mmol) was added to a stirred solution of compound 1 (50 mg, 0.12 mmol) in DMF at RT. The resulting reaction mixture was stirred at RT for 16 hours. The reaction was monitored by TLC and LC-MS. The reaction mixture was purified by reverse phase column chromatography to yield compound 3 as a pale brown, sticky liquid (30 mg, yield 43%).

For step 2, compound 4 (32.61 mg, 0.034 mmol) was added to a stirred solution of compound 3 (20 mg, 0.034 mmol) in a mixture of DCM (0.8 mL) and EtOH (0.02 mL) (25:1). The resulting reaction mixture was stirred at RT for 16 hours. The reaction was monitored by LC-MS. The reaction mixture was concentrated under reduced pressure to yield crude compound. The crude compound was diluted with acetone and filtered, the filtrate was concentrated under reduced pressure and triturated with MTBE, followed by lyophilization to yield Photocatalyst 9 as a yellow solid (24 mg, yield 54%). Photocatalyst 9 is an example photocatalyst in accordance with the present disclosure.

### Example 10. Preparation of Photocatalyst 10

The description of preparation of a photocatalyst is described with reference to Scheme 14:

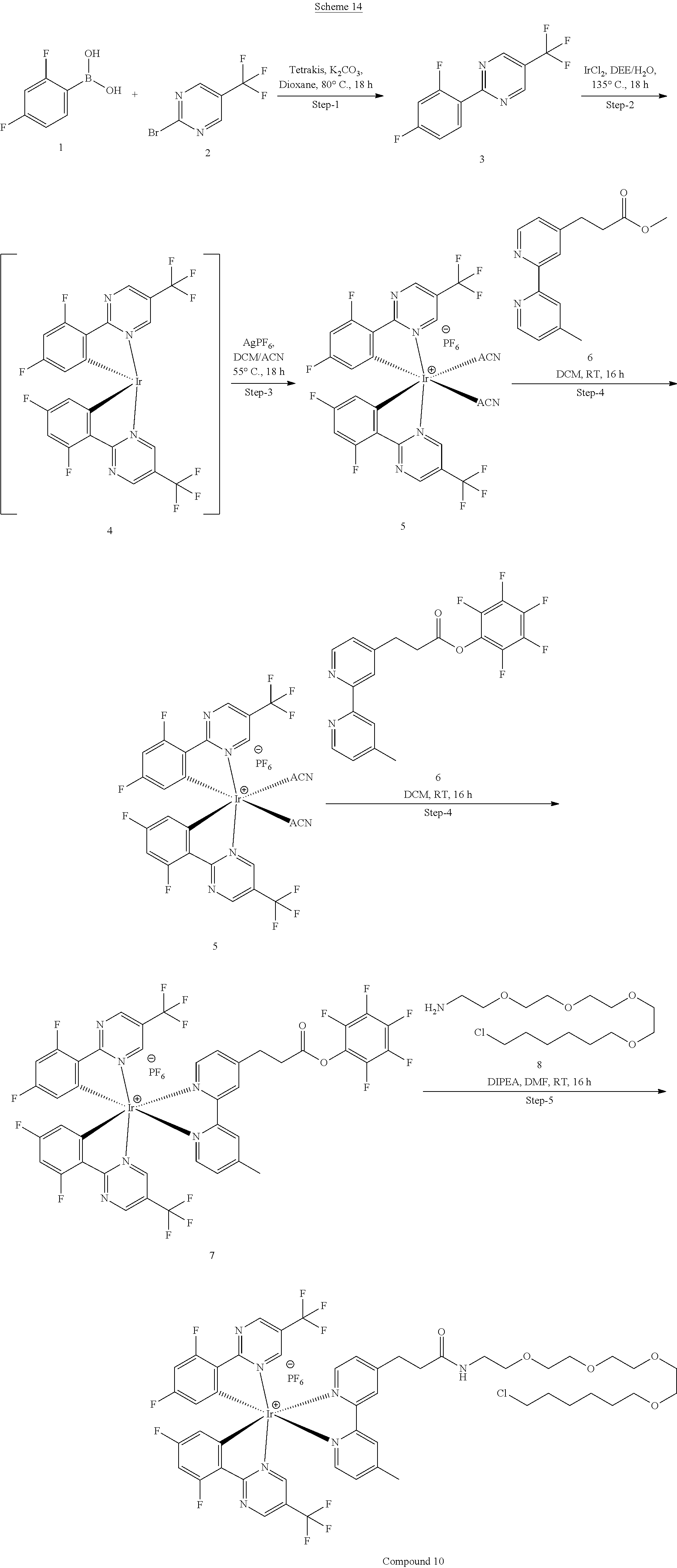

With reference to Scheme 14, step 1, K₂CO₃ (1.8 g, 12.00 mmol, 3 equivalents) was added to a stirred solution of (2,4-difluorophenyl) boronic acid (1.0 g, 4.40 mmol, 1.0 equivalent) and 2-bromo-5-(trifluoromethyl) pyrimidine (1.3 g, 8.80 mmol, 2 equivalents) in 1,4 dioxane (10 mL). The mixture was purged with argon gas for 10 minutes. Pd(PPh₃)₄ was added and the mixture was heated to 80° C. for 16 hours. After completion of the reaction, the reaction mixture was filtered through a celite bed, and filtrate was concentrated under reduced pressure to yield crude product. The crude compound was purified by column chromatography using petroleum ether and ethyl acetate as eluent. The required fractions were concentrated under reduced pressure to yield 2-(2,4-difluorophenyl)-5-(trifluoromethyl) pyrimidine (compound 3, 0.8 g, 70%) as a white solid.

For step 2, IrCl₃·H₂O (600 mg, 2.009 mmol, 1 equivalent) and compound 8 (1.145 g, 4.421 mmol, 2.2 equivalents) were added to a RBF and evacuated and refilled with N₂ three times. Then a degassed solution of 2-ethoxy ethanol (24 mL) and water (8.4 mL) was added and heated to 120° C. for 16 hours. The progress of the reaction was monitored by LC-MS. After completion of the reaction, the reaction mixture was cooled to RT. The yellow precipitate was filtered, washed with water (120 mL) and hexane (50 mL) and dried under vacuum to yield compound 4 (605 mg, yield 33%) as yellow solid.

For step 3, AgPF₆ (143 mg, 0.564 mmol, 2.1 equivalents) was added to a stirred solution of compound 4 (400 mg, 0.268 mmol, 1 equivalents) in acetonitrile (30 mL) at RT and stirred for 16 hours. The progress of the reaction was monitored by LC-MS. After completion of the reaction, the reaction mixture was concentrated under reduced pressure and crude compound was purified by triturating with diethyl ether (20 mL) to yield compound 5 (350 mg, 55.7%) as a pale-yellow solid.

For step 4, compound 6 (0.11 g, 0.269 mmol, 1 equivalent) was added to a solution of compound 5 (0.17 g, 0.269 mmol, 1 equivalent) in DCM (5 mL) at RT, and stirred for 16 hours. The progress of the reaction was monitored by LC-MS and TLC (70% ethyl acetate in petroleum ether). After completion of the reaction, the reaction mixture was concentrated under reduced pressure to yield crude compound. The crude compound was stirred in pentane (10 mL) and filtered to yield compound 7 (0.2 g, yield 83%) as a yellow solid.

For step 5, compound 8 (NH₂-PEG4-Halolinker, 0.05 g, 0.356 mmol, 2 equivalents) and DIPEA (0.1 mL, 0.496 mmol, 3 equivalents) was added to a solution of compound 7 (0.2 g, 0.175 mmol, 1 equivalent) in DMF (2 mL) at RT. The mixture was stirred for 16 hours. The progress of the reaction was monitored by LC-MS and TLC (10% MeOH in DCM). After completion of the reaction, the reaction mixture was concentrated under reduced pressure to yield crude compound. The crude compound was purified by silica gel (NH Silicagel mesh) column chromatography by eluting with mobile phase 0-5% methanol in DCM. The required fractions were concentrated under reduced pressure to yield Photocatalyst 10 (0.035 g, yield 20%) as a brown gummy compound. The compound was purified by pre-LC. Photocatalyst 10 is an example photocatalyst in accordance with the present disclosure.

### Example 11. Photo-Responsive Labeling within Live Cells

Initially, reagents were prepared. An Ir—Cl photocatalyst master stock solution was prepared at 10 mM in 100% DMSO. The specific Ir—Cl photocatalyst used in this example was the photocatalyst prepared in accordance with Example 2. To create a working solution for the Ir—Cl catalyst, a portion of the Ir—Cl catalyst master stock solution was diluted in 1×DPBS, such that the final Ir—Cl catalyst concentration was 10 μM and the DMSO concentration was 0.1%. A labeling agent master stock was also prepared, having a 25 mM concentration in 100% DMSO. The particular labeling agent used was the one prepared in accordance with Example 1. To create a working solution for the labeling agent, a portion of the labeling agent master stock solution was diluted in 1×DPBS, such that the final concentration of labeling agent was 250 μM and the DMSO concentration was 1%.

A human KRAS (G12DHaloTag®/+) cell line (HD-103-021, Horizon Discovery) with protein reporter was grown to 80-90% confluence. Cells were harvested and pelleted down by centrifuging at 1000 g, for 5 minutes at 4° C. (Eppendorf Centrifuge 5427 R). Supernatant was discarded and the cell pellet was resuspended in ice cold 1×DPBS (Gibco™) and mixed. Subsequently, cells were again pelleted down by centrifuging at 1000 g, for 5 minutes at 4° C. Supernatant was discarded and fresh ice cold 1×DPBS was added to the pellet and mixed.

For each experimental sample set, approximately 7×10⁶ cells were taken. Ir—Cl catalyst working solution was added to the cell pellet, and the pellet was dislodged and mixed. Cells were incubated with the Ir—Cl catalyst for 30 minutes at 4° C. with continuous end-to-end rotation (15 rpm, TARSONS-ROTOSPIN). After incubation, cells were pelleted down by centrifuging at 1000 g, for 5 minutes at 4° C. Supernatant was discarded and 1×DPBS was added to the pellet. Pellet was re-suspended and mixed, followed by centrifugation at 1000 g, for 5 min. at 4° C.

Supernatant was discarded and pellet was re-suspended in labeling agent working in 1×DPBS (final 1% DMSO concentration). The pellet was dislodged, mixed and immediately irradiated with light of wavelength 450 nm for 10 minutes at 100% power, using the M2 photo-reactor (Penn PhD Photoreactor M2 from Sigma Aldrich). After irradiation, cells were pelleted down by centrifuging at 1000 g, for 5 minutes at 4° C.

Supernatant was discarded and the pellet was re-suspended in 1× cell lysis buffer (Cell Signaling Technology) supplemented with protease-phosphatase inhibitor (Halt™ Protease and Phosphatase Inhibitor Cocktail (100×)) and mixed briefly. This mixture was then incubated on ice for 15 minutes.

After incubation, the cell lysate was sonicated using a probe-sonicator (SONICS vibra-cell Fisher Scientific) with 15 s on, 10 s off, at 30% amplitude for 3 cycles. The cell lysate was centrifuged at 18,000 g at 4° C. for 15 minutes. Supernatant was collected and subsequently taken for immunoprecipitation (IP) by coating with specific antibody (HALO-tagged proteins-HALO-tag IP) and biotinylated proteins (Streptavidin magnetic bead-based IP) in accordance with Example 12 and Example 13, respectively.

### Example 12. Immunoprecipitation of HALO-Tagged Proteins (K-Ras-Interacting Partner Proteins)

50 μl of Dynabead™ magnetic bead slurry (Dynabeads™ Protein G Thermo Fisher Scientific) was aliquoted. The beads were collected against magnetic stand and washed three times with wash buffer (PBST). The beads were then collected against a magnetic stand and supernatant was discarded.

To coat the beads with antibody, Anti-HaloTag® Monoclonal Antibody from Promega was added to PBST at a final concentration of 1:50. Beads were incubated with antibody for 1 hour at RT with continuous end-to-end rotation. Beads were then collected against a magnetic rack and washed three times with wash buffer.

Following this, beads were incubated with pre-clearing solution (5% BSA, 2.5 M carbonic anhydrase) for 1 hour with continuous end-to-end rotation at RT. The beads were washed three times with wash buffer to remove excess bound antibody.

Cell lysates obtained in accordance with Example 11 were added to the anti-HaloTag antibody-coated beads and mixed. The lysate and beads were incubated for 3 hours at RT with continuous end-to-end rotation. After incubation, the tubes were placed on a magnetic rack and beads were collected. Supernatant was discarded. The beads were subsequently washed three times with wash buffer.

To elute, 50 μl SDS-Laemmli buffer (with 1:10 1 M DTT) was added to the beads and mixed. This mixture was held at 95° C. for 10 minutes. Tubes were then placed on a magnetic rack, and supernatant, representing final pull-down eluate sample, was collected in respective tubes.

The pull-down eluate samples were then subjected to a Western blot. 10 μl of the pull-down eluate sample were loaded per lane in 4-15% Tris-Glycine gel (Bio-Rad). Gel electrophoresis was run at 80 V for an initial 10 minutes, followed by 100 V until the dye front reached the end of the gel.

Proteins were transferred from gel to nitrocellulose membrane using the Bio-Rad Wet Transfer apparatus at 4° C., at a constant current of 300 mA for 90 minutes. The membrane was rinsed once with TBST and blocked with 5% milk in TBST for 60 minutes at RT. After blocking, membrane was washed with TBST and a primary antibody specific to one of the potential K-Ras interacting partner proteins was added (1:1000) and kept for overnight at 4° C. room on a shaker. The next day, the membrane was washed 3 times with TBST, with an incubation of 10 minutes per wash, at RT.

A HRP-tagged secondary antibody specific to the primary antibody was added and incubated for 120 minutes at RT with constant shaking. After incubation, membrane was washed with TBST (for 10 minutes, 3 times). Following this, membrane was developed using chemiluminescence reagent (Clarity™ Western ECl Substrate) in the Bio-Rad ChemiDoc imaging system.

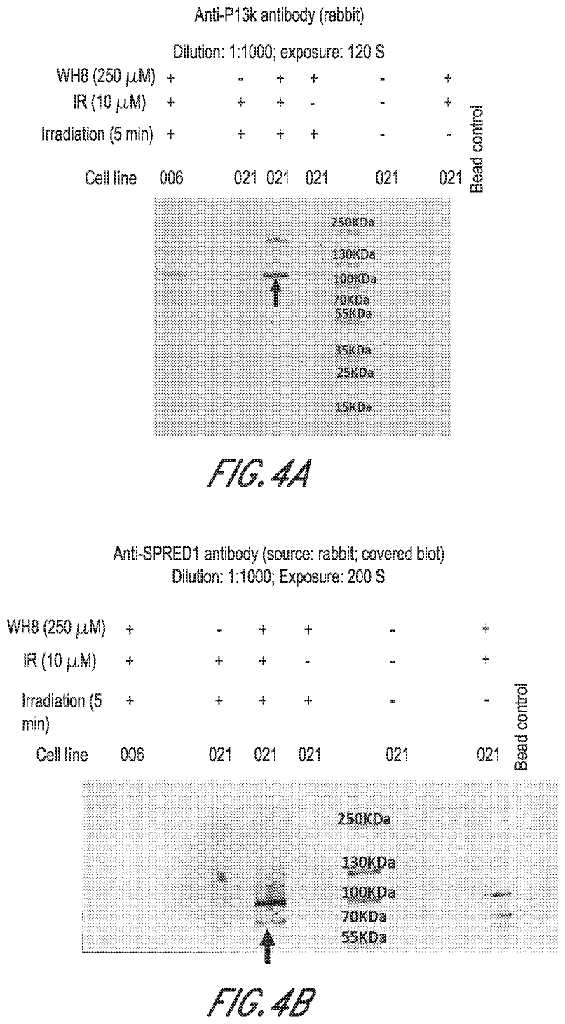

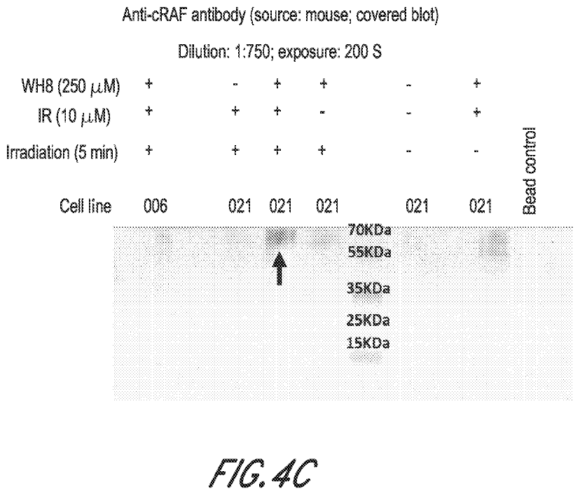

FIG. 4A is an image of Western blot results in accordance with the above procedure, using Anti-PI3k primary antibody. Each column of the gel represents a different condition. For instance, the presence of labeling agent (WH8), presence of photocatalyst (IR), and irradiation were varied for each column. Additionally, the cell line used (006 or 021) was varied for one of the columns. The condition that included labeling agent, photocatalyst, and irradiation of cell line 021 resulted in a distinct band of PI3 Kinase P110α. Without being bound to a particular theory, it is believed that the higher molecular weight bands observed suggests the formation of aggregates including PI3 Kinase P110α.

FIG. 4B is an image of Western blot results in accordance with the above procedure, using anti-SPRED1 antibody. As with FIG. 4A, the various combinations represent combinations of labeling agent, photocatalyst, irradiation, and cell line. The condition including a labeling agent, photocatalyst, irradiation, and cell line 021 resulted in the formation of distinct bands. Without being bound to a particular theory, it is believed that the higher molecular weight bands observed suggests the formation of aggregates including SPRED1.

FIG. 4C is an image of Western blot results in accordance with the above procedure, using anti-cRAF antibody. As with FIG. 4A, the various combinations represent combinations of labeling agent, photocatalyst, irradiation, and cell line. The condition including a labeling agent, photocatalyst, irradiation, and cell line 021 resulted in the formation of a distinct band.

### Example 13. Immunoprecipitation of Biotinylated Proteins (Biotinylated K-Ras Interactor Partner Proteins)

50 μl of Pierce Streptavidin magnetic bead slurry was aliquoted, and beads were collected against magnetic stand and washed three times with wash buffer (PBST). The beads were incubated with pre-clearing solution (5% BSA, 2.5 μM carbonic anhydrase) for 1 hour with continuous end-to-end rotation at RT. The beads were then collected against magnetic rack and washed three times with wash buffer.

The cell lysates obtained according to Example 11 were added to the Streptavidin beads and mixed. This mixture was incubated for 3 hours at RT with continuous end-to-end rotation. After incubation, tubes were placed on magnetic rack and beads were collected. The supernatant was discarded. The beads were then washed three times with wash buffer.

To elute, 50 μl of SDS-Laemmli buffer (with 1:10 1 M DTT) was added to the beads and mixed. This mixture was held at 95° C. for 10 minutes. The tubes were then placed on magnetic rack, supernatant was collected in respective tubes. These were the final pull-down eluate samples.

The pull-down eluate samples were then subjected to Western blotting. 10 μl of pull-down eluate was loaded per lane in 4-15% Tris-Glycine gel (Bio-Rad). Gel was run at 80V for an initial 10 minutes, followed by 100V until the dye front reaches the end of the gel.

Proteins were transferred from gel to nitrocellulose membrane using the Bio-Rad Wet Transfer apparatus at 4° C., at a constant current of 300 mA for 90 minutes. The membrane was rinsed once with TBST and blocked with 5% milk in TBST for 60 minutes at RT. After blocking, membrane was washed with TBST and requisite antibody was added (1:1000) and kept for overnight at 4° C. on a shaker. The next day, the membrane was washed 3 times with TBST, with an incubation of 10 minutes per wash, at RT.

Respective HRP-tagged secondary antibody was added and kept for 120 minutes at RT with constant shaking. After 120 minutes incubation, membrane was washed with TBST (10 minutes wash for 3 times). Following this, the membrane was developed using chemiluminescence reagent (Clarity™ Western ECl Substrate) in the Bio-Rad ChemiDoc imaging system.

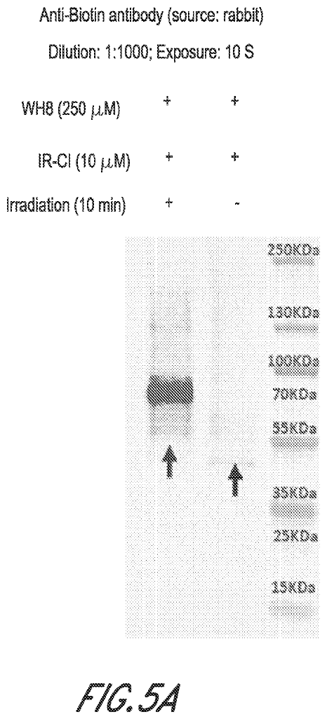

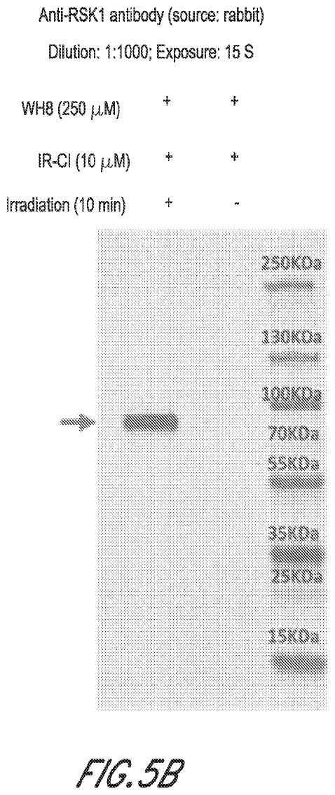

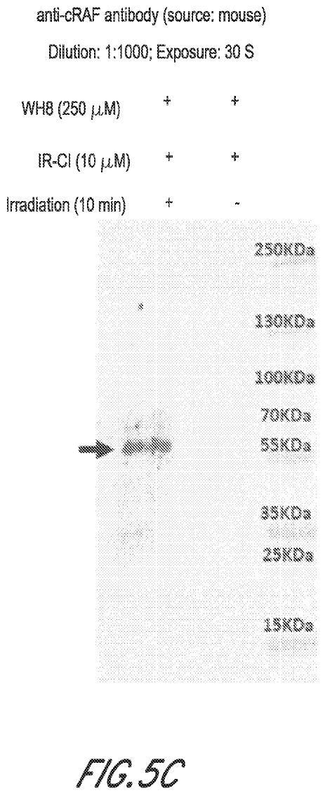

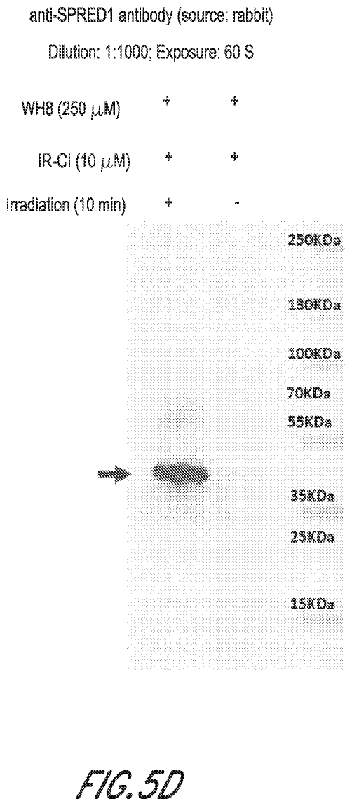

FIG. 5A is an image of Western blot results in accordance with the above procedure, using anti-biotin primary antibody. Each column of the gel represents a different condition with respect to irradiation: the left column represents cells that experienced 10 minutes of irradiation whereas, for the right column, the cells did not experience irradiation. There are noticeable bands starting at about the 55 kDa mark and upward for the irradiated cells. The non-irradiated cells show only faint bands, suggesting some level of nonspecific binding.

FIG. 5B is an image of Western blot results in accordance with the above procedure, using anti-RSK1 antibody. As with FIG. 5A, the two columns in the gel represent an irradiated or non-irradiated cells. There is a distinct band of expected molecular weight for the irradiated cells, but no visible bands for the non-irradiated cells.

FIG. 5C is an image of Western blot results in accordance with the above procedure, using anti-cRAF antibody. As with FIG. 5A, the two columns in the gel represent an irradiated or non-irradiated cells. There is a distinct band of expected molecular weight for the irradiated cells, but no visible bands for the non-irradiated cells.

FIG. 5D is an image of Western blot results in accordance with the above procedure, using anti-cRAF antibody. As with FIG. 5A, the two columns in the gel represent an irradiated or non-irradiated cells. There is a distinct band of expected molecular weight for the irradiated cells, but no visible bands for the non-irradiated cells.

FIG. 5E is an image of Western blot results using cell lysates and an anti-GAPDH antibody. As with FIG. 5A, the two columns in the gel represent an irradiated or non-irradiated cells. There is a distinct band of expected molecular weight for the both the irradiated cells and non-irradiated cells. This confirms that the blots depicted in FIGS. 5A-5D had similar numbers of cells in the irradiated and non-irradiated conditions.

FIG. 5F is an image of Western blot results in accordance with the above procedure with an anti-SOS1 antibody. For this gel, three types of cells were run: KRAS (G12DHaloTag®/+) protein reporter (HD-103021), KRAS HaloTag protein reporter control (HD-103-037), and SW48 Parental (HD-PAR-006). For the KRAS (G12DHaloTag®/+) protein reporter cells with photocatalyst (IR-Cl), labeling partner (WH8), and irradiation, there is a distinct SOS1 band observed, indicated by the arrow. For the two control conditions (HD-103-037 and HD-PAR-006), no distinct band was observed. Notably, other methods of detecting protein associations have previously failed to identify SOS1 as a protein associated with KRas.

### Example 14. Fusion of HaloTag with Cereblon does not Block Cereblon's Ubiquitination of Ikaros

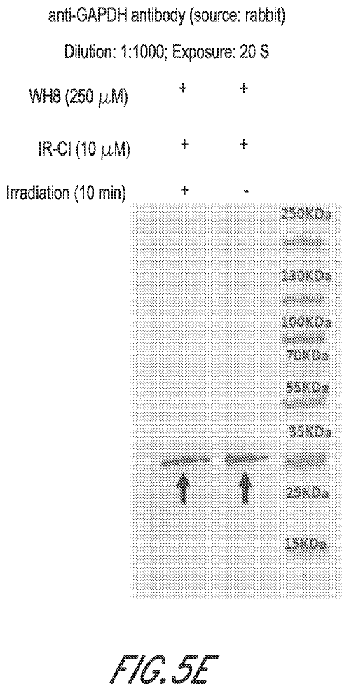

A stable cell line, using Jurkat cells, was created where native cereblon (CRBN) was knocked out and a fusion of HaloTag protein and CRBN was expressed (referred to herein as “the HALO.CRBN Jurkat cell line”). CRBN is known to ubiquitinate ikaros (IKZF1) in the presence of lenalidomide and/or pomalidomide. FIG. 6 is an image of Western blot results using lysates of the HALO.CRBN Jurkat cell line, the cells having been exposed to various concentrations of pomalidomide or lenalidomide. The gel was stained using mouse anti-IKZF1 antibody. The dilution was 1:1000 and the exposure was about 10 seconds. The 10 M pomalidomide and 10 M lenalidomide lanes include observably less IKZF1 than the control or 1 μM pomalidomide lanes, suggesting that the HaloTag/cereblon fusion protein can ubiquitinate IKZF1 in the presence of lenalidomide and/or pomalidomide.

### Example 15. Detecting Cereblon Interactions

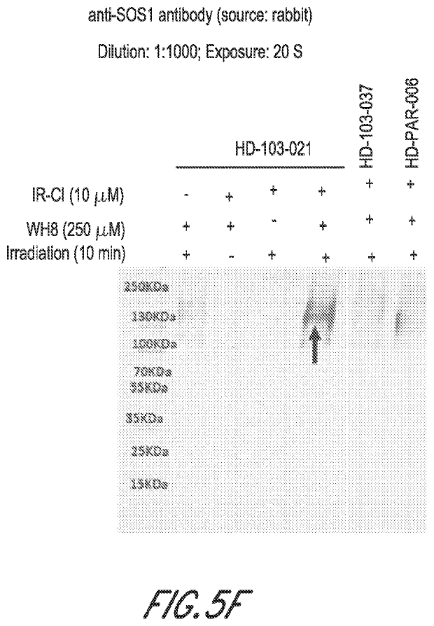

The HALO.CRBN Jurkat cell line of Example 14 was used to generate cell lysates for a series of gels shown in FIGS. 7A-13B for examining interactions between CRBN and other proteins.

The HALO.CRBN Jurkat cells were grown in suspension to 80-90% confluence. The cells were either (1) treated with 10 μM pomalidomide for 16 hours, (2) treated with 10 μM lenalidomide for 16 hours, or (3) left untreated.

Cells were harvested and pelleted down by centrifuging at 1000 g, for 5 minutes at 4° C. (Eppendorf Centrifuge 5427 R). Supernatant was discarded and the cell pellet was resuspended in ice cold 1×DPBS (Gibco™) and mixed. Subsequently, cells were again pelleted down by centrifuging at 1000 g, for 5 minutes at 4° C. Supernatant was discarded and fresh ice cold 1×DPBS was added to the pellet and mixed. For each experimental sample set, approximately 7×10⁶ cells were taken.

An Ir—Cl photocatalyst master stock solution was prepared at 10 mM in 100% DMSO. The particular Ir—Cl photocatalyst used in this example was the photocatalyst prepared in accordance with Example 2. To create a working solution for the Ir—Cl catalyst, a portion of the Ir—Cl catalyst master stock solution was diluted in 1×DPBS, such that the final Ir—Cl catalyst concentration was 10 μM and the DMSO concentration was 0.1%. A labeling agent master stock was also prepared, having a 25 mM concentration in 100% DMSO. The particular labeling agent used was the one prepared in accordance with Example 1. To create a working solution for the labeling agent, a portion of the labeling agent master stock solution was diluted in 1×DPBS, such that the final concentration of labeling agent was 250 μM and the DMSO concentration was 1%.

The Ir—Cl catalyst working solution was added to the cell pellet, and the pellet was dislodged and mixed. Cells were incubated with the Ir—Cl catalyst for 30 minutes at 4° C. with continuous end-to-end rotation (15 rpm, TARSONS-ROTOSPIN). After incubation, cells were pelleted down by centrifuging at 1000 g, for 5 minutes at 4° C. Supernatant was discarded and 1×DPBS was added to the pellet. Pellet was re-suspended and mixed, followed by centrifugation at 1000 g, for 5 min. at 4° C. Supernatant was discarded and pellet was re-suspended in labeling agent working in 1×DPBS (final 1% DMSO concentration). The pellet was dislodged, mixed and immediately irradiated with light of wavelength 450 nm for 10 minutes at 100% power, using the M2 photo-reactor (Penn PhD Photoreactor M2 from Sigma Aldrich). After irradiation, cells were pelleted down by centrifuging at 1000 g, for 5 minutes at 4° C.

Supernatant was discarded and the pellet was re-suspended in 1× cell lysis buffer (Cell Signaling Technology) supplemented with protease-phosphatase inhibitor (Halt™ Protease and Phosphatase Inhibitor Cocktail (100×)) and mixed briefly. This mixture was then incubated on ice for 15 minutes.

After incubation, the cell lysate was sonicated using a probe-sonicator (SONICS vibra-cell Fisher Scientific) with 15 s on, 10 s off, at 30% amplitude for 3 cycles. The cell lysate was centrifuged at 18,000 g at 4° C. for 15 minutes. The supernatant was collected and subsequently taken for protein content estimation (Pierce™ BCA Protein Assay Kit). Then, supernatant containing approximately 200 g of protein was used for each IP.

For IP, 50 μl of streptavidin magnetic bead slurry (Pierce Streptavidin Magnetic Beads, ThermoFisher Scientific) was aliquoted. The beads were collected against magnetic stand and washed three times with wash buffer (PBST). The beads were then collected against a magnetic stand and supernatant was discarded.

Following this, beads were incubated with pre-clearing solution (5% BSA, 2.5 M carbonic anhydrase) for 1 hour with continuous end-to-end rotation at RT. The beads were washed three times with wash buffer.

Cell lysates obtained from the irradiated HALO.CRBN Jurkat cells as described above were added to the streptavidin beads and mixed. The lysate and beads were incubated for 3 hours at RT with continuous end-to-end rotation. After incubation, the tubes were placed on a magnetic rack and beads were collected. Supernatant was discarded. The beads were subsequently washed three times with wash buffer.

To elute, 50 μl SDS-Laemmli buffer (with 1:10 1 M DTT) was added to the beads and mixed. This mixture was held at 95° C. for 10 minutes. Tubes were then placed on a magnetic rack, and supernatant, representing final pull-down eluate sample, was collected in respective tubes.

The pull-down eluate samples were then subjected to a Western blot. 10 μl of the pull-down eluate sample were loaded per lane in 4-15% Tris-Glycine gel (Bio-Rad). Gel electrophoresis was run at 80 V for an initial 10 minutes, followed by 100 V until the dye front reached the end of the gel.

Proteins were transferred from gel to nitrocellulose membrane using the Bio-Rad Wet Transfer apparatus at 4° C., at a constant current of 300 mA for 90 minutes. The membrane was rinsed once with TBST and blocked with 5% milk in TBST for 60 minutes at RT. After blocking, membrane was washed with TBST and a primary antibody specific to one of the potential CRBN-interacting partner proteins was added (1:1000) and kept for overnight at 4° C. room on a shaker. The next day, the membrane was washed 3 times with TBST, with an incubation of 10 minutes per wash, at RT.

A respective HRP-tagged secondary antibody, specific to the primary antibody, was added and incubated for 120 minutes at RT with constant shaking. After incubation, membrane was washed with TBST (for 10 minutes, 3 times). Following this, membrane was developed using chemiluminescence reagent (Clarity™ Western ECl Substrate) in the Bio-Rad ChemiDoc imaging system. These images are shown in FIGS. 7A-12B.

FIGS. 7A and 7B are images of gels run using the lysates and stained using anti-CK1 polyclonal rabbit antibody. The gels shown in FIGS. 7A and 7B were stained at a dilution of 1:1000. Exposure for the gel of FIG. 7A was about 20 seconds whereas exposure for the gel of FIG. 7B was about 60 seconds. There are observable CK1 bands in lanes 7 and 8, corresponding to immunoprecipitated lysate from cells with photocatalyst, labeling agent, and irradiation (light), suggesting that CK1 was proximally labeled with the labeling agent, thereby indicating protein-protein interaction between CRBN and CK1.

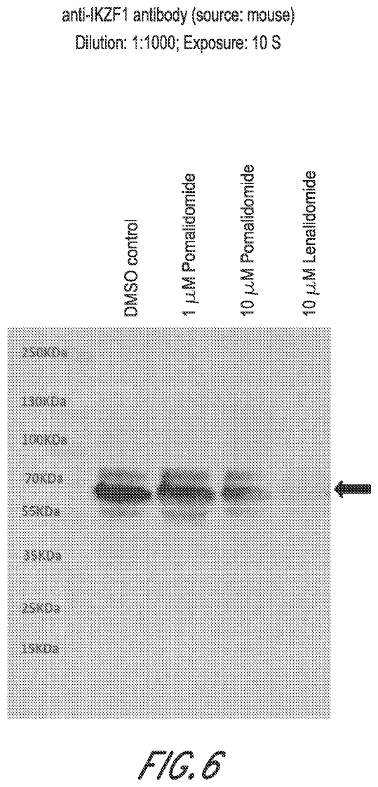

FIGS. 8A and 8B are images of gels run using the immunoprecipitated proteins and stained using anti-SALL4 monoclonal rabbit antibody. The gels shown in FIGS. 8A and 8B were stained at an antibody dilution of 1:500. Exposure for the gel of FIG. 8A was about 10 seconds whereas exposure for the gel of FIG. 8B was about 30 seconds. There are observable SALL4 bands in lanes 7 and 8, corresponding to immunoprecipitated lysate from cells with photocatalyst, labeling agent, and irradiation (light), suggesting that SALL4 was proximally labeled with the labeling agent, thereby indicating protein-protein interaction between CRBN and SALL4.

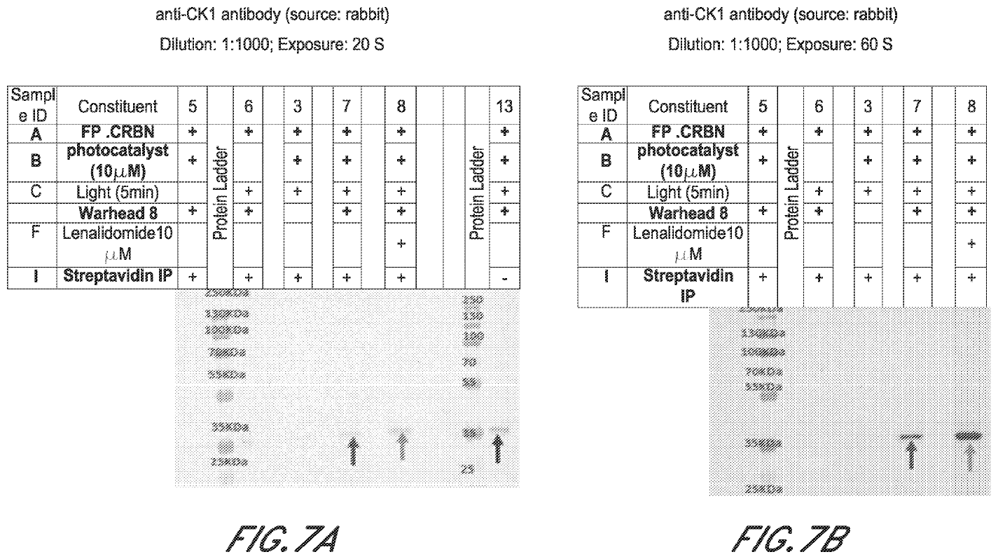

FIG. 9 is an image of Western blot results using the lysates and stained using anti-KEAP1 antibody at 1:1000 dilution. The exposure was about 30 seconds. There are observable KEAP1 bands in lanes 13 and 1, corresponding to lysate from cells with photocatalyst, labeling agent, and irradiated (light) cell lysate, though there were no observable KEAP1 bands within the immunoprecipitation pull-down lanes (5-8). This suggests that KEAP1 protein had not been proximally labeled and thus that there was no protein-protein interaction between CRBN and KEAP1 in the absence of a molecular glue agent.

FIG. 10 is an image of Western blot results using the lysates and stained using anti-β-tubulin rabbit antibody at 1:1000 dilution. The exposure was about 5 seconds. There are observable β-tubulin bands in lanes 13 and 1, corresponding to lysate from cells with photocatalyst, labeling agent, and irradiated (light) cell lysate, though there were no observable β-tubulin bands within the immunoprecipitation pull-down lanes (5-8). This suggests that 3-tubulin protein had not been proximally labeled and thus that there was no protein-protein interaction between CRBN and β-tubulin in the absence of a molecular glue agent.

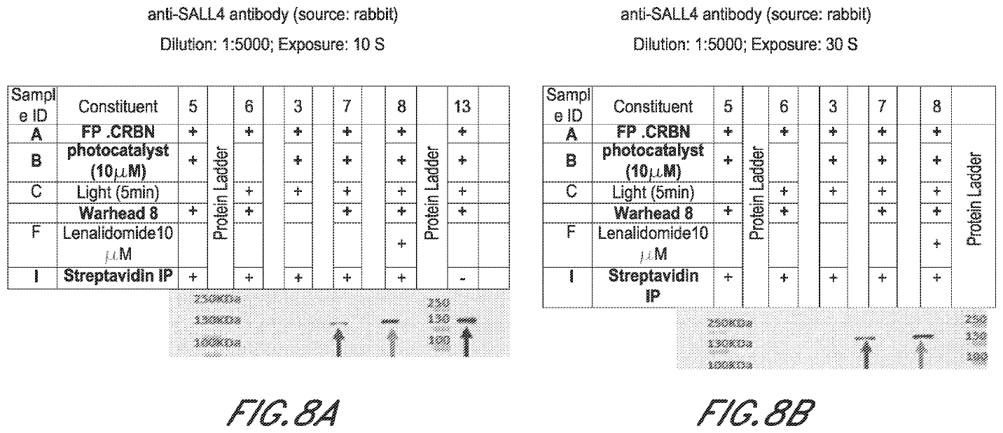

FIGS. 11A and 11B are images of gels run using the lysates and stained using anti-PARP1 polyclonal rabbit antibody. The gels shown in FIGS. 11A and 11B were stained at an antibody dilution of 1:1000. Exposure for the gel of FIG. 11A was about 20 seconds whereas exposure for the gel of FIG. 11B was about 120 seconds. There are observable PARP1 bands in lanes 13 and 1, corresponding to lysate from cells with photocatalyst, labeling agent, and irradiated (light) cell lysate, though there were no observable PARP1 bands within the Immunoprecipitation pull-down lanes (5-8). This suggests that PARP1 protein had not been proximally labeled and thus that there was no protein-protein interaction between CRBN and PARP1 in the absence of a molecular glue agent.

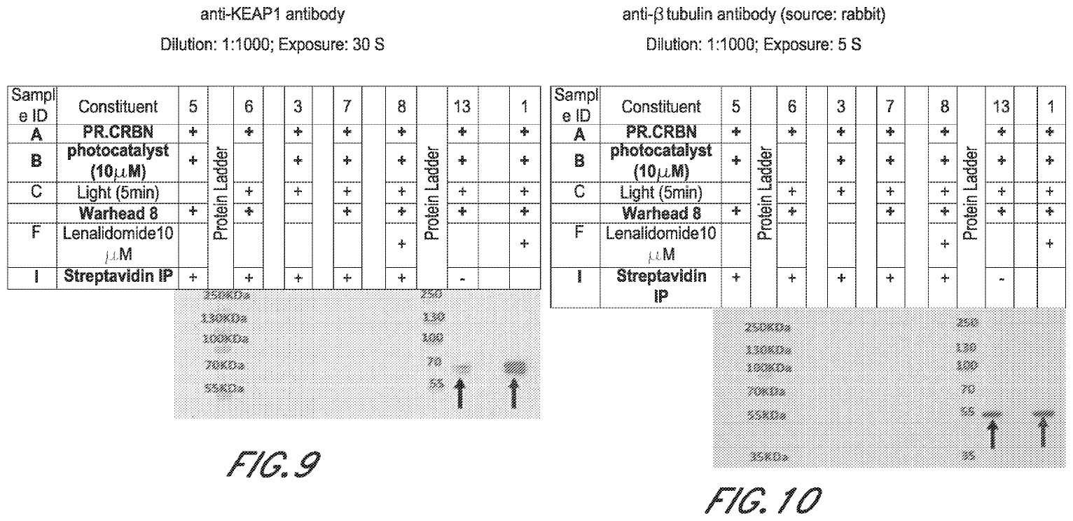

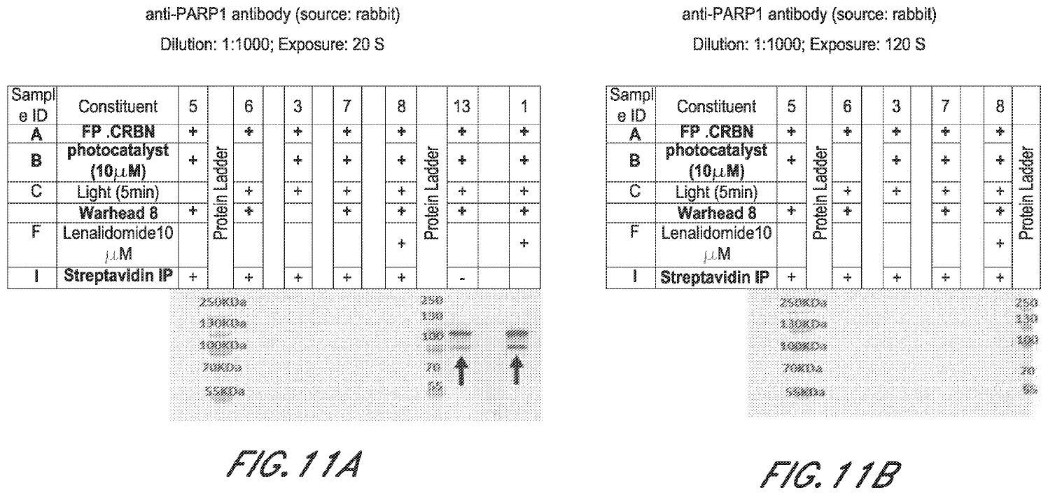

FIGS. 12A and 12B are images of gels run using the lysates and stained using an antibody specific to a known oncogenic protein. The gels shown in FIGS. 12A and 12B were stained at an antibody dilution of 1:1000. Exposure for the gel of FIG. 12A was about 45 seconds whereas exposure for the gel of FIG. 12B was about 60 seconds. There are observable bands corresponding to the oncogenic protein in lanes 7 and 8, corresponding to lysate from cells with photocatalyst, labeling agent, and irradiation (light), suggesting that the oncogenic protein was proximally labeled with the labeling agent, thereby indicating protein-protein interaction between CRBN and the oncogenic protein.

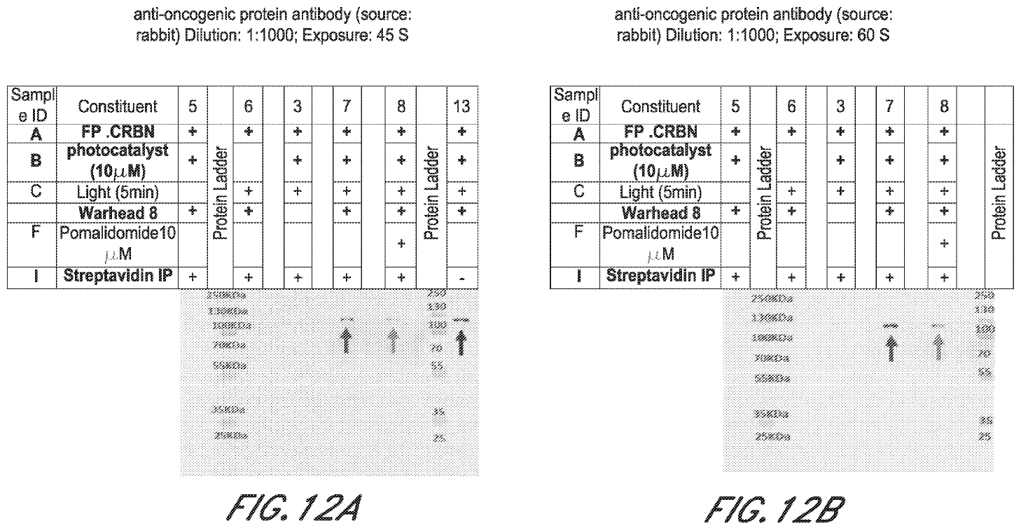

FIGS. 13A and 13B are images of gels run using the lysates and stained using anti-IKZF1 monoclonal mouse antibody. The gels shown in FIGS. 13A and 13B were stained at an antibody dilution of 1:100. Exposure for the gel of FIG. 13A was about 10 seconds whereas exposure for the gel of FIG. 13B was about 30 seconds. There are observable IKZF1 bands in lanes 7 and 8, corresponding to immunoprecipitated lysate from cells with photocatalyst, labeling agent, and irradiation (light), suggesting that IKZF1 was proximally labeled with the labeling agent, thereby indicating protein-protein interaction between CRBN and IKZF1.

### Example 16. A Proximity-Based Tagging Approach was Able to Detect K-Ras-Associated Proteins not Detected by an Alternative Approach

A photo-responsive, proximity-based tagging approach disclosed herein was compared to an existing approach, immunoprecipitation pull-down, for detecting protein interactions. Two cell lines were used. The first is a stable cell line expressing a HaloTag/K-Ras G12D fusion protein (Horizon Discovery, Catalog Number HD-103-021). The second, is a stable cell line expressing K-Ras G12D and HaloTag separately (Horizon Discovery, Catalog Number HD-103-0037).

Cells were grown to 80-90% confluence. Cells were rinsed using 1×PBS followed by addition of 0.25% Trypsin-EDTA. Cells were then incubated for about 2 min. at 5% CO2, 37° C. Once cells had dislodged, the Trypsin was neutralized with complete media. Cells were pelleted down by centrifuging at 1000 g, for 5 min., at 4° C. (Eppendorf Centrifuge 5427 R). The supernatant was discarded, and the cell pellet was re-suspended in ice cold 1×DPBS (Gibco™) and mixed. Following this, cells were again pelleted down by centrifuging at 1000 g, for 5 min., at 4° C. Supernatant was discarded and fresh ice cold 1×DPBS was added to the pellet and mixed.

For each experimental sample set, 7×10⁶ cells were taken. 10 M Ir—Cl catalyst (compound 1, see Scheme 1) was added to the cell pellet, and the pellet was dislodged and mixed. Cells were incubated with the Ir—Cl catalyst for 30 min. at 4° C. with continuous end-to-end rotation at 15 rpm (TARSONS-ROTOSPIN). After incubation, cells were pelleted down by centrifuging at 1000 g, for 5 min., at 4° C. Supernatant was discarded and 1×DPBS was added to the pellet. The pellet was re-suspended and mixed, followed by centrifugation at 1000 g, for 5 min., at 4° C. Cell pellet re-suspended in 900 μl 1×PBS and transferred to 6-well plate. A labeling agent accordance with Example 1 was added to the cell suspension at a final concentration of 250 μM in 1×DPBS (final 1% DMSO concentration).

A 6-well plate with the cells were placed in a CLIGHT photo-reactor. Generally, irradiation was carried out for 5 min. For SOS1, a 10 min. irradiation was performed.

After irradiation, the cell suspensions were transferred to microfuge tubes and were pelleted down by centrifuging at 1000 g, for 5 min., at 4° C. The supernatant was discarded and the pellet was re-suspended in 1× cell lysis buffer (Cell Signaling Technology) supplemented with protease-phosphatase inhibitor (Halt™ Protease and Phosphatase Inhibitor Cocktail (100×), sodium fluorite, PMSF) and mixed briefly. This was incubated on ice for 15 min.

After incubation, lysate was sonicated using probe-sonicator (SONICS vibra-cell Fisher Scientific) with 15 s ON, 10 s OFF, at 30% amplitude for 3 cycles. The cell lysate was centrifuged at 18,000 g at 4° C. for 15 min. The supernatant, including the cell lysate, was collected in tubes.

Protein content of the lysates was estimated (Pierce™ BCA Protein Assay Kit). The cell lysates were then taken for immunoprecipitation (IP) by coating with specific antibody (HALO-tag IP) and biotinylated proteins (Streptavidin magnetic bead-based IP).

### HALO-Tag IP

50 μl of Dynabead™ magnetic bead slurry (Dynabeads™ Protein G Thermo Fisher Scientific) were aliquoted. The beads were collected against a magnetic stand and washed three times with wash buffer (PBST). After the washes, the beads were collected against magnetic stand and supernatant was discarded.

To coat beads with antibody, Anti-HaloTag® Monoclonal Antibody from Promega (source: mouse) was added to PBST at a final concentration of 1:50. The beads were incubated with antibody for 1 hour at RT with continuous end-to-end rotation. The beads were then collected against the magnetic rack and washed three times with wash buffer. Following this, the beads were incubated with a pre-clearing solution (5% BSA, 2.5 μM carbonic anhydrase) for 1 hour with continuous end-to-end rotation at RT. The beads were washed three times with wash buffer to remove excess bound antibody.

200 μg of each irradiated cell lysate obtained as described above were added to the HALO antibody-coated beads and mixed. The lysate and bead mixture was incubated for 3 hours at RT with continuous end-to-end rotation.

After incubation, tubes were placed on magnetic rack and beads were collected. The supernatant was discarded. The beads were washed three times with wash buffer. 50 μl SDS-Laemmli buffer (with 1:10 1 M DTT) was added to the beads and mixed. This mixture was heated to 95° C. for 10 min. The tubes were then placed on a magnetic rack to isolate the beads, and supernatant was collected in respective tubes. The collected supernatants were the subsequently used for Western blotting.

### Streptavidin Bead IP

50 μl of Pierce Streptavidin magnetic bead slurry was taken, beads were collected against magnetic stand and washed three times with wash buffer (PBST). The beads were incubated with pre-clearing solution (5% BSA, 2.5 μM carbonic anhydrase) for 1 hour with continuous end-to-end rotation at RT. The beads were collected against a magnetic rack and washed three times with wash buffer. Cell lysates obtained from the irradiated cells were added to the Streptavidin beads and mixed. The lysate and bead mixture was incubated for 3 hours at RT with continuous end-to-end rotation.

After incubation, tubes were placed on magnetic rack and beads were collected. Supernatant was discarded. Beads were washed three times with wash buffer. 50 μl of SDS-Laemmli buffer (with 1: 10 1 M DTT) was added to the beads and mixed before being heated to 95° C. for 10 min. Tubes were placed on a magnetic rack and supernatant was collected in respective tubes. The collected supernatants were subsequently used for Western blotting.

### Western Blot

The eluate samples from the HALO-tag IP and streptavidin bead IP were subjected to Western blotting. 10 μl of pull-down eluate samples were loaded per lane in a 4-15% Tris-Glycine gel (BIO-RAD). The gel was run at 80V initially for 10 min., then at 100V till the dye front reached the end of the gel. The proteins within the gel were transferred from to nitrocellulose membrane using the Bio Rad Wet Transfer apparatus at 4° C., at a constant current of 300 mA for 90 min.

The membrane was rinsed once with TBST and blocked with 5% milk in TBST for 60 minutes at RT. After blocking, membrane was washed with TBST and a primary antibody specific to one of the potential KRAS-interacting partner proteins was added at 1:1000 and kept overnight at 4° C. on a shaker.

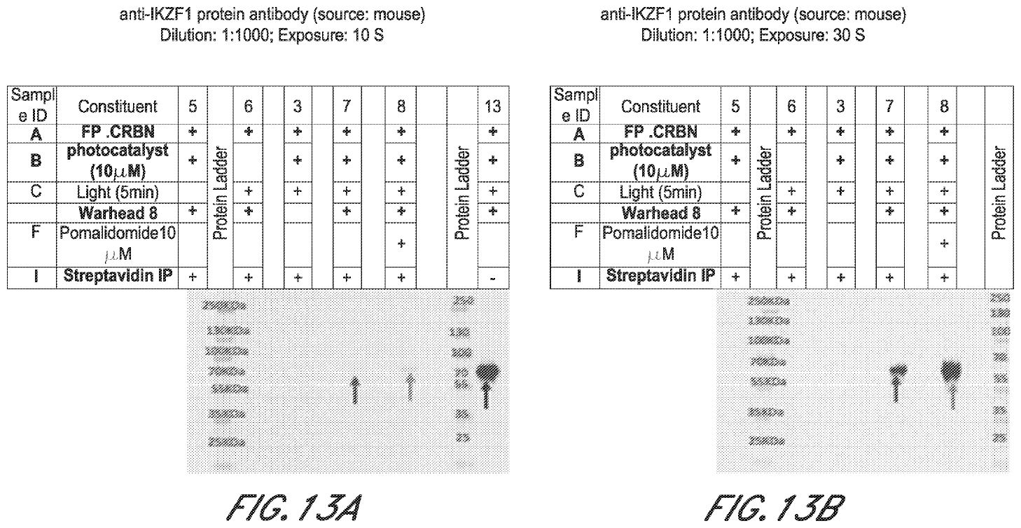

The next day, the membrane was washed with TBST (10 minutes 3× times) at RT. The respective HRP-tagged secondary antibody specific to the primary antibody was added and kept for 120 minutes at RT with constant shaking. After, the membrane was washed with TBST (for 10 minutes, 3 times). The membrane was developed using chemiluminescence reagent (Immobilon® Forte Western HRP Substrate) in the BIO-RAD ChemiDoc imaging system to create the FIGS. 14A-17B.

FIGS. 14A and 14B show gels stained with anti-cRAF antibodies.

For FIG. 14A, the rabbit anti-cRAF antibody was diluted to 1:750. The exposure was about 60 seconds. The lysates were subjected to the Halo-Tag IP, discussed above. The gel shows a weak cRAF band in lane 5, corresponding to the immunoprecipitation with Halo antibody while total lysate lanes 6 and 7 show distinct cRAF bands.

For FIG. 14B, the gel was stained with rabbit anti-cRAF antibody diluted 1:1000. The exposure was about 30 seconds. The lysates were subjected to the Streptavidin bead-based IP, discussed above. The gel shows a distinct cRAF band in lane 5, corresponding to the immunoprecipitation with a photocatalyst, irradiation, and labeling agent. The total lysate lanes 6 and 7 also show distinct cRAF bands. This demonstrates that the approach described herein was more effective in identifying protein-protein interactions than immunoprecipitation pull-down.

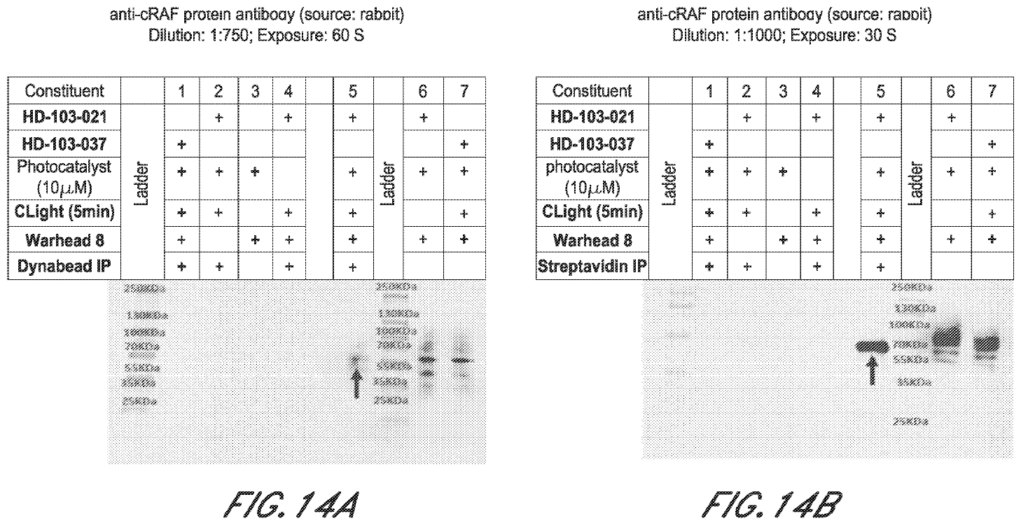

FIGS. 15A and 15B show gels stained with anti-RSK1 antibodies.

For FIG. 15A, the rabbit anti-RSK1 antibody was diluted to 1:1000. The exposure was about 90 seconds. The lysates were subjected to the Halo-Tag IP, discussed above. The gel shows no observable band in lane 5, corresponding to the immunoprecipitation with Halo antibody while total lysate lanes 6 and 7 show distinct RSK1 bands. A longer exposure of about 180 seconds also did not result in observable bands.

For FIG. 15B, the gel was stained with rabbit anti-RSK1 antibody diluted 1:1000. The exposure was about 30 seconds. The lysates were subjected to the Streptavidin bead-based IP, discussed above. The gel shows a distinct RSK1 band in lane 5, corresponding to the immunoprecipitation with a photocatalyst, irradiation, and a labeling agent. The total lysate lanes 6 and 7 also show distinct RSK1 bands. The results depicted in FIGS. 15A and 15B indicate that the approach disclosed herein was capable of identifying RSK1 in proximity with K-Ras even though the immunoprecipitation pull-down approach could not.

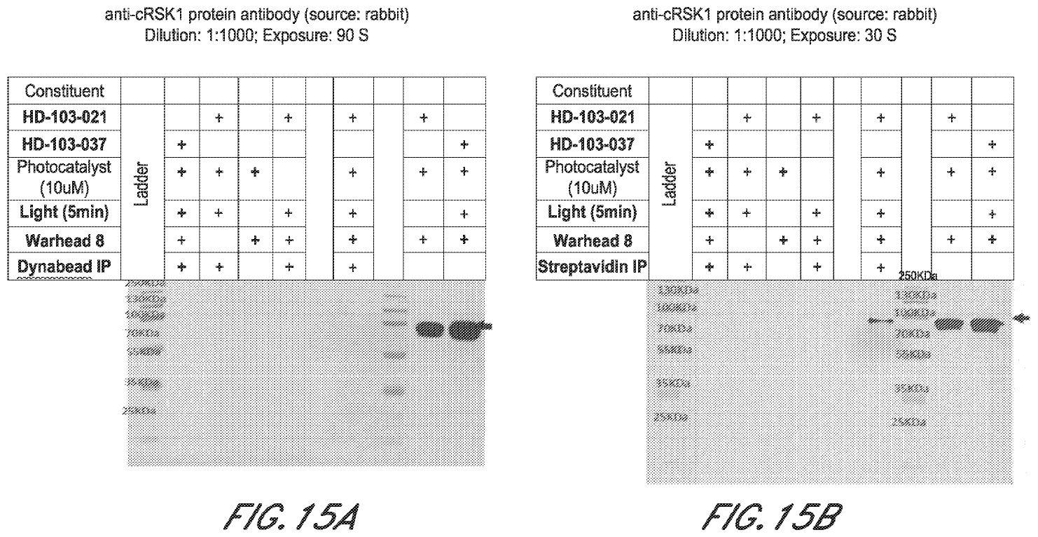

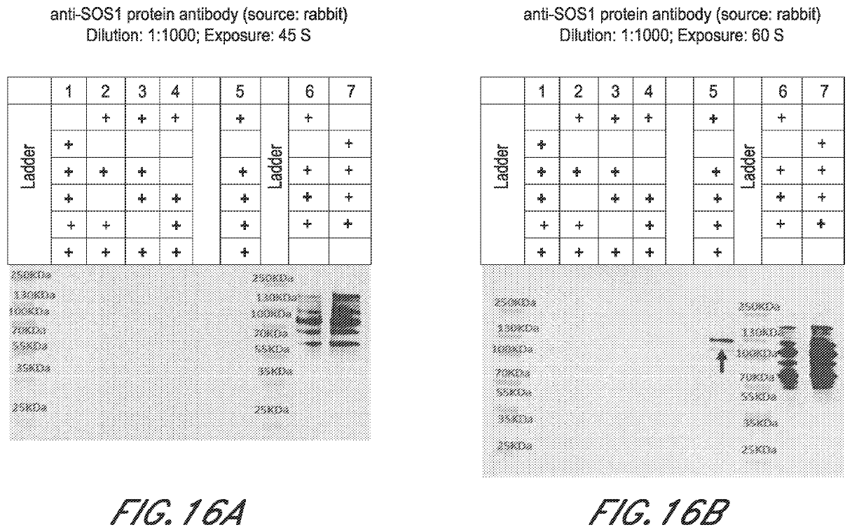

FIGS. 16A and 16B show gels stained with anti-SOS1 antibodies.

For FIG. 16A, the rabbit anti-SOS1 antibody was diluted to 1:1000. The exposure was about 45 seconds. The lysates were subjected to the Halo-Tag IP, discussed above. The gel shows no observable band in lane 5, corresponding to the immunoprecipitation with Halo antibody while total lysate lanes 6 and 7 show distinct SOS1 bands.

For FIG. 16B, the gel was stained with rabbit anti-SOS1 antibody diluted 1:1000. The exposure was about 60 seconds. The lysates were subjected to the Streptavidin bead-based IP, discussed above. The gel shows a distinct SOS1 band in lane 5, corresponding to the immunoprecipitation with a photocatalyst, irradiation, and a warhead. The total lysate lanes 6 and 7 also show distinct RSK1 bands. The results depicted in FIGS. 16A and 16B indicate that the approach disclosed herein was capable of identifying SOS1 in proximity with K-Ras even though the immunoprecipitation pull-down approach could not.

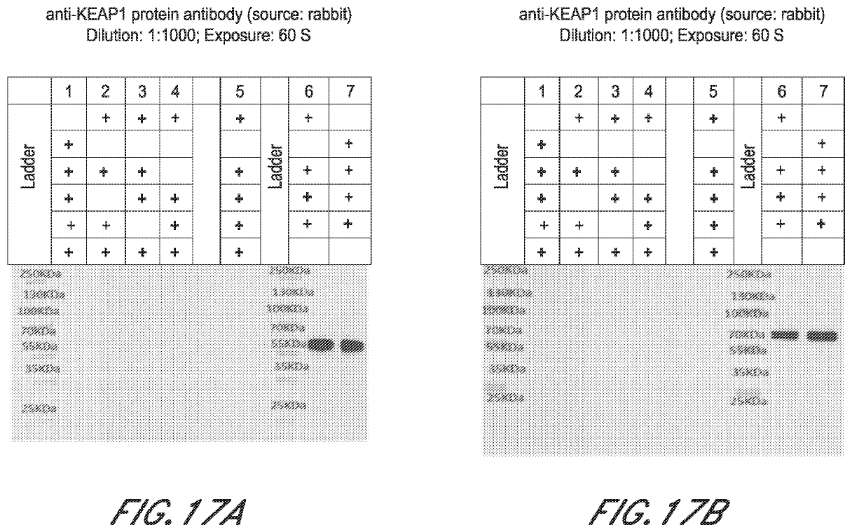

FIGS. 17A and 17B show gels stained with anti-KEAP1 antibodies.

For FIG. 17A, the rabbit anti-KEAP1 antibody was diluted to 1:1000. The exposure was about 60 seconds. The lysates were subjected to the Halo-Tag IP, discussed above. The gel shows no observable band in lane 5, corresponding to the immunoprecipitation with Halo antibody while total lysate lanes 6 and 7 show distinct SOS1 bands.

For FIG. 17B, the gel was stained with rabbit anti-KEAP1 antibody diluted 1:1000. The exposure was about 60 seconds. The lysates were subjected to the Streptavidin bead-based IP, discussed above. The gel shows no observable band in lane 5, corresponding to the immunoprecipitation with a photocatalyst, irradiation, and a labeling agent. The total lysate lanes 6 and 7 show distinct KEAP1 bands. These results suggest no protein-protein interaction between KRAS G12D and KEAP 1.

### Example 17. Proximal Labeling of KRAS Interactors May be Dependent on Irradiation Time

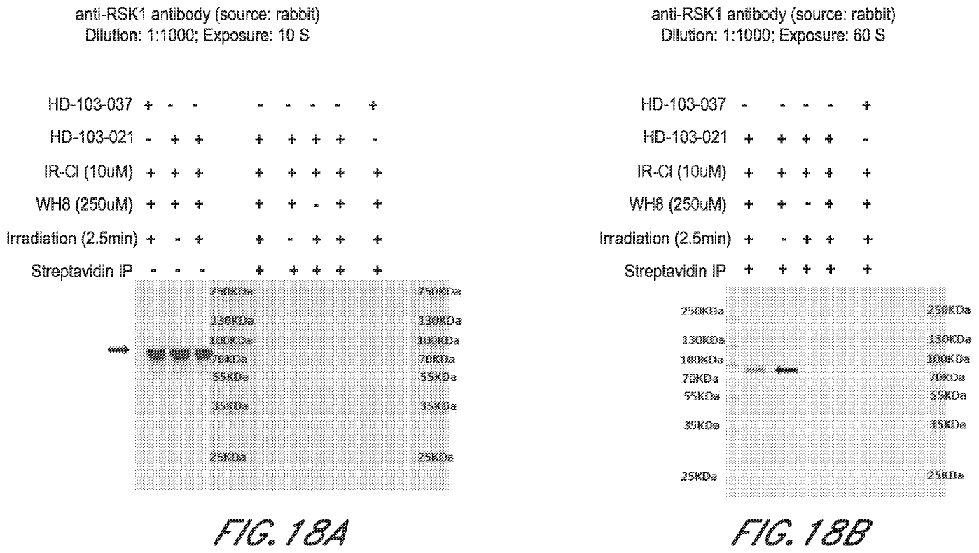

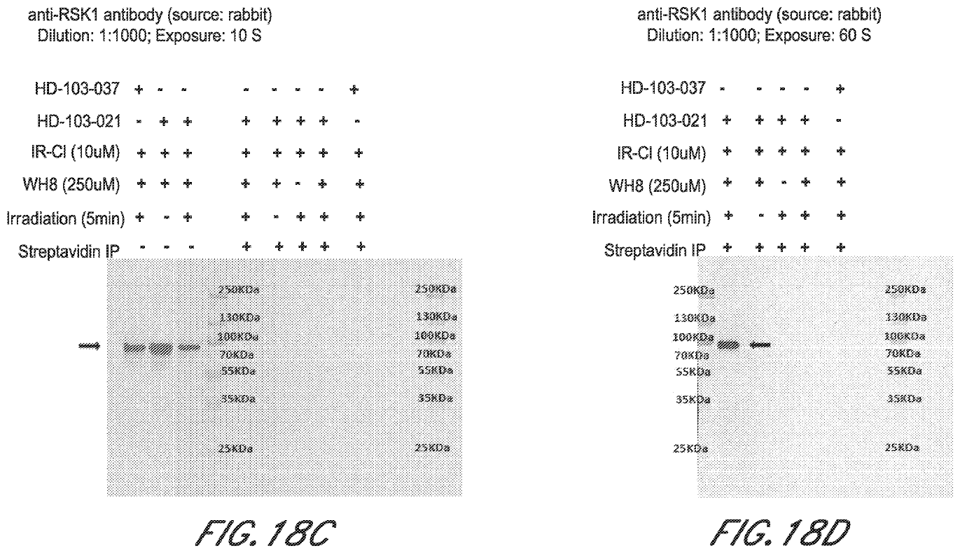

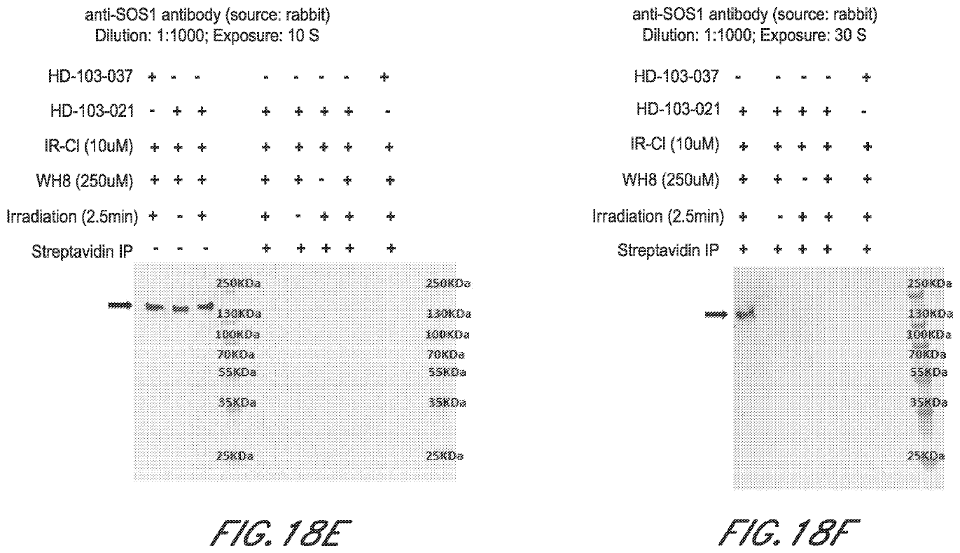

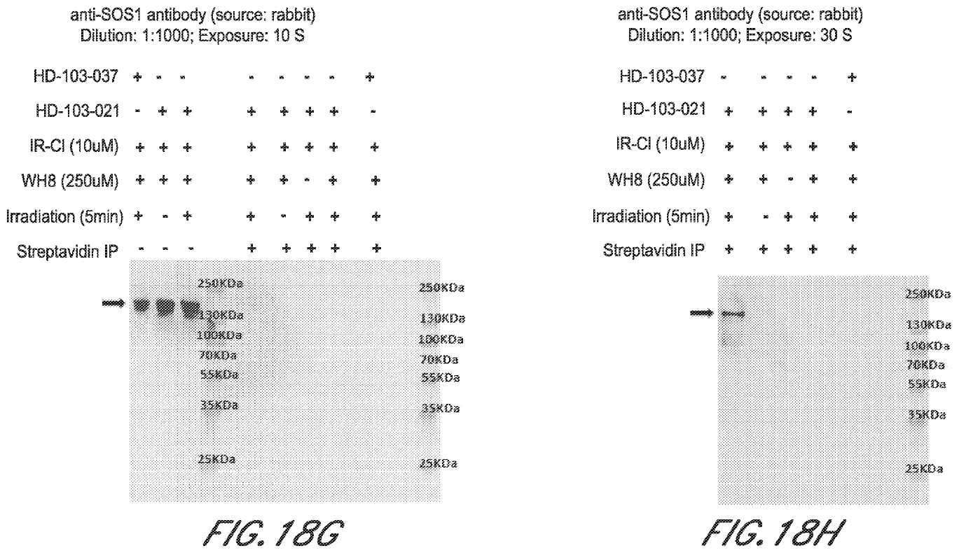

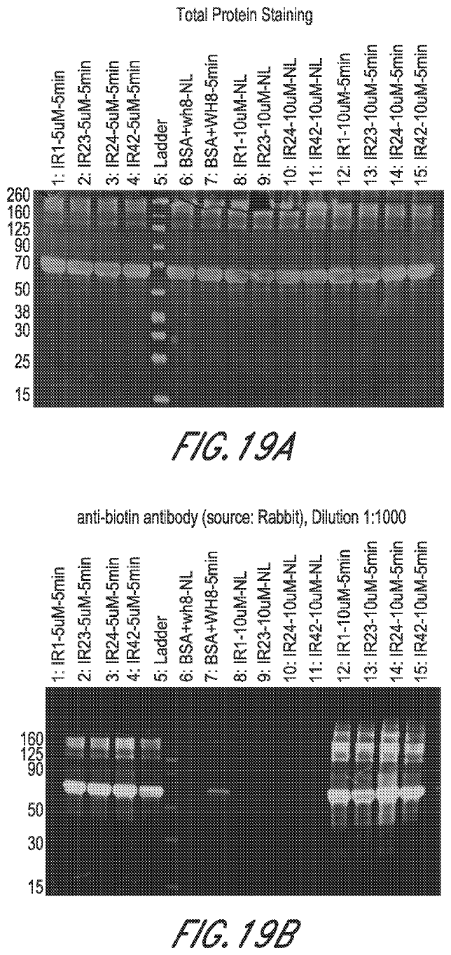

FIGS. 18A-18D are images of Western blots using both the lysates and streptavidin pull-down proteins which were stained using anti-RSK1 polyclonal rabbit antibody. The antibody dilution was 1:1000. Exposure for the gel of FIGS. 6A and 6C was about 10 seconds whereas exposure for the gel of FIGS. 6B and 6D was about 60 seconds. There are observable RSK1 bands in lanes 1 of FIGS. 6B and 6D, corresponding to immunoprecipitated lysate from cells with photocatalyst, labeling agent, and irradiation (light). Presence of these bands suggests that RSK1 was proximally labeled with the labeling agent. The observed RSK1 band is thicker for the 5-minute irradiation sample (FIG. 6D) than 2.5-minute irradiation sample (FIG. 6B). Without being bound to a particular theory, it is believed that these results indicate that the proximal labeling of the KRAS interactors may be dependent upon irradiation time.

FIGS. 18E-18H are images of Western blot using both the lysates and streptavidin pull-down proteins which were stained using anti-SOS1 polyclonal rabbit antibody. The antibody dilution was 1:1000. Exposure for the gels of FIGS. 6E and 6G was about 10 seconds whereas exposure for the gels of FIGS. 6F and 6H was about 30 seconds. There are observable SOS1 bands in lane 1 of FIGS. 6F and 6H, corresponding to immunoprecipitated lysate from cells with photocatalyst, labeling agent, and irradiation (light). Presence of these bands suggest that SOS1 was proximally labeled with the labeling agent. The observed SOS1 band is thicker for the 5-minute irradiation (FIG. 6H) than for the 2.5-minute irradiation sample (FIG. 6F). Without being bound to a particular theory, it is believed that these results indicate that the proximal labeling of the KRAS interactors may be dependent upon irradiation time.

### Example 18. System for Testing Ir-Photocatalyst Activity

### Catalyst-Dependent Biotinylation of Bovine Serum Albumin

Photocatalyst was combined with diazirine labeling compounds 8 or 9 and BSA in DPBS to yield reaction mixtures with 200 uL total solution volume. BSA concentration was 10 uM; diazirine labeling compound was fixed as 100 uM; the final catalyst concentration was set as 5 uM and 10 uM respectively. These samples were then either placed in the dark or irradiated with visible (450 nm) light in a M2 photoreactor for 5 minutes at 100% intensity. 60 uL samples were then removed, combined with 20 μL of 4× reducing SDS-Laemmli buffer (with 1:10 1 M DTT), mixed and heated at 95° C. for 5 minutes. 15 μL of each sample was then analyzed by Fluorescent western blot.

### Fluorescent Western Blot

The sample was loaded into a 4-20% Tris-Glycine gel (Bio-Rad). Gel was run at 120V for 80 minutes or until the dye front reaches the end of the gel. Proteins were transferred from gel to nitrocellulose membrane using the iBlot 2 semi-dry Transfer apparatus. The membrane was used to detect total protein for normalization which follow the manufacturer's manual (Revert™ 700 Total Protein Stain, LI-COR). After that, the membrane rinsed once with TBST and blocked with h Intercept® Blocking Buffer for 1 hour at RT with gentle shaking. After blocking, membrane was washed with TBST and requisite antibody was added (1:1000) and kept for overnight at 4° C. on a shaker. The next day, the membrane was washed 3 times with TBST, with an incubation of 10 minutes per wash, at RT. IRDye® 800CW secondary antibodies or IRDye® 680RD secondary antibodies was added and kept for 1 hour at RT with constant shaking. After 1 hour incubation, the membrane was washed with TBST (10 minutes wash for 3 times). Following this, the membrane was scanned on an Odyssey Imager (LI-COR Odyssey M Imaging System).

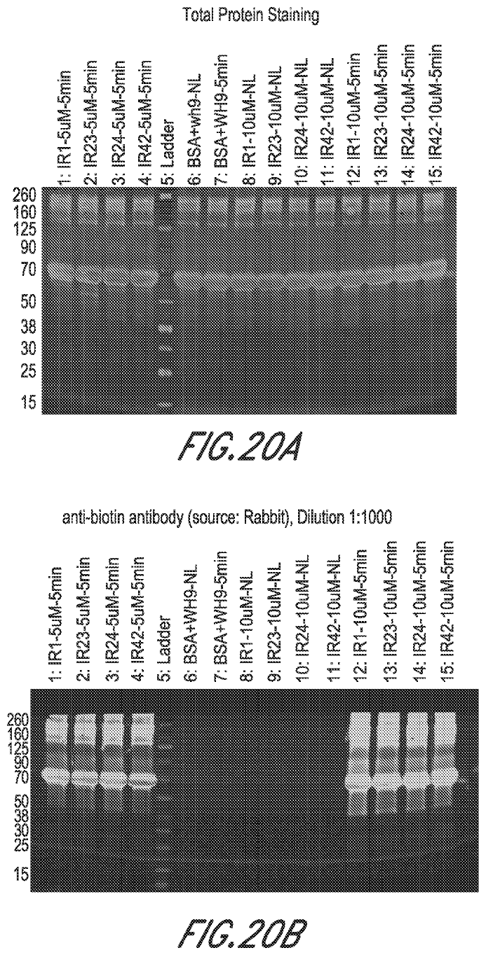

FIGS. 19A-19B are Western blot results showing biotinylation of BSA by different photocatalysts combined with labeling reagent WH8. FIG. 19A is the total protein stain results for all the treatment conditions. The same amount of BSA was used in each treatment. FIG. 19B is the fluorescent Western blot results using rabbit anti-biotin antibody. There appears to be no band for samples that did not receive irradiation (lanes 6 and 8-11). There appear to be biotinylated BSA bands in both the 5 μM and 10 μm catalyst treatment for samples subjected to 5 minutes irradiation.

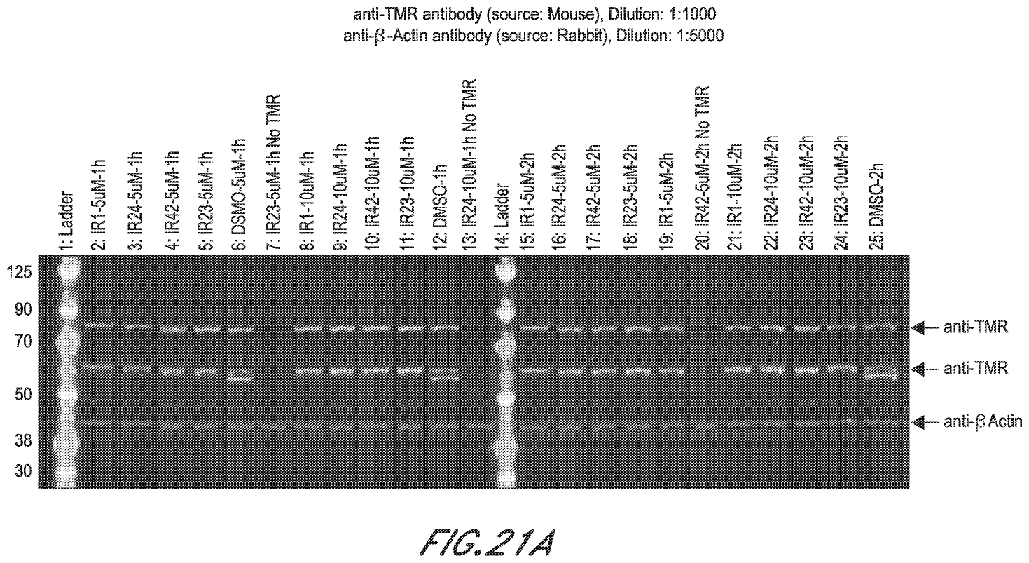

FIGS. 20A-20B are Western blot results showing biotinylation of BSA by different photocatalysts combined with labeling reagent WH9. FIG. 20A shows the total protein stain results for all the treatment conditions. The same amount of BSA was used in each treatment. FIG. 20B shows the fluorescent Western blot results using rabbit anti-Biotin antibody. There appears to be no band for samples that did not receive irradiation (lanes 6 and 8-11). There appear to be biotinylated BSA bands in both the 5 μM and 10 um catalyst treatment for samples subjected to 5 minutes irradiation.

### Example 19. Ir-Photocatalyst Cell Permeability

Photo-responsive labeling within cells was carried out as described similar to the procedure as described in Example 11. An Ir-photocatalyst master stock solution was prepared at 10 mM or 5 mM in 100% DMSO. KRAS (G12DHaloTag®/+) cells (HD-103-021, Horizon Discovery) were grown to 90% confluency in 6-well cell culture dishes using phenol-free RPMI1640 media (Gibco™). Photocatalysts IR1(photocatalyst 1), IR23 (photocatalyst 3), IR24 (photocatalyst 4), IR42 (photocatalyst 5), or DMSO were added to the appropriate wells for 5 or 10 μM final photocatalyst concentrations. The cells were incubated for 1 or 2 hours at 37° C. The media was then aspirated and cells were gently washed twice with 2 mL fresh RPMI 1640, with an incubation of 10 minutes per wash at 37° C. TAMRA-Cl fluorescent dye (Promega, 68251) was diluted with OptiMEM to a 5 μM working solution. 0.8 mL of the working dye solution was added to the cells, and allowed to incubate for 15 minutes at 37° C. The cells were gently washed twice with 2 mL cold DPBS, aspirated, then added 200 μL RIPA supplemented with protease-phosphatase inhibitor (Halt™ Protease and Phosphatase Inhibitor Cocktail (100×)) and mixed briefly. The cell lysate was collected into 1.5 mL tubes. The cell lysate was sonicated using a probe-sonicator (SONICS vibra-cell Fisher Scientific) with 15 s on, 10 s off, at 30% amplitude for 3 cycles. The cell lysate was centrifuged at 18,000 g at 4° C. for 20 minutes. Supernatant was collected and concentration was adjusted to 1 mg/ml via BCA assay (Pierce™ BCA Protein Assay Kit, Thermofisher). Cell lysates were processed and analyzed by fluorescent Western blot with anti-TAMRA and anti-β-Actin (for normalization) antibodies. Without being bound to a particular theory, TAMRA signal may be inversely proportional to Ir binding to intracellularly-expressing Halotag-KRAS(G12D), and therefore may be inversely proportional to Ir cell permeability.

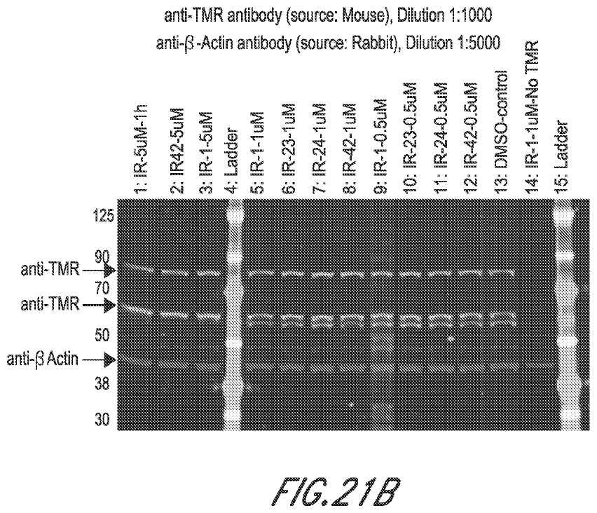

FIGS. 21A and 21B are Halo-chaser assay fluorescent Western blot results with anti-TAMRA antibody and anti-β-Actin antibody. The mouse anti-TAMRA antibody was diluted to 1:1000. The rabbit anti-β-Actin antibody was diluted to 1:5000, corresponding to the loading control. Photocatalysts IR1, IR23, IR24, and IR 42 were used.

For FIG. 21A, sample was treated with the catalyst for 1 hour or 2 hours. For the 1 hour samples, compared to the DMSO control, IR1-5 μM and IR1-10 μM showed a little less binding to the Halo tag, while the other three photocatalysts (IR23, IR24, and IR42) appear to completely bind the Halo tags. Without being bound to a particular theory, these results may indicate that IR24, IR42, and IR23 have higher cell permeability than IR1.

For FIG. 21B, it appears that 0.5 μM and 1 μM photocatalyst concentrations did not fully bind the Halotag. 5 μM of IR42 showed complete binding. 5 μM of IR1 showed majority binding. Without being bound to a particular theory, these results appear to indicate that IR42 has a higher cell permeability than IR1.

What is claimed is:

1. A method of proximity-based labeling of intracellular molecules, comprising:
intracellularly introducing a binding agent complex to a cell, wherein the binding agent complex comprises a protein of interest coupled to a binding agent, wherein the binding agent is capable of binding to a catalyst complex;
introducing the catalyst complex to the cell, thereby forming a biomolecular antenna comprising the catalyst complex and the binding agent complex;
introducing a labeling agent comprising a label moiety and a reactive moiety, to the cell, wherein the reactive moiety is configured to be activated to a reactive state by the catalyst complex; and
activating the catalyst complex, thereby activating the labeling agent by transfer of energy from the catalyst complex to the reactive moiety and causing the labeling agent to bind to a biomolecule within the cell.

2. The method of claim 1, wherein introducing the binding agent complex to the cell comprises introducing a nucleotide construct to the cell, wherein the nucleotide construct encodes for the binding agent complex, and wherein the nucleotide construct can be expressed by the cell.

3. The method of claim 2, wherein introducing a nucleotide construct to a cell comprises transfecting the cell.

4. The method of claim 1, wherein the biomolecule is an intracellular protein, a peptide, a chromatin, or a nucleic acid.

5. The method of claim 1, further comprising imaging a signal from the label moiety.

6. The method of claim 1, wherein the catalyst complex comprises a photocatalyst.

7. The method of claim 6, wherein the photocatalyst comprises a transition metal complex.

8. The method of claim 7, wherein activating the transition metal complex comprises photoactivating the transition metal complex.

9. The method of claim 8, said photoactivating comprising shining light on the transition metal complex, wherein the light has a wavelength from about 380 nm to about 700 nm.

10. The method of claim 8, wherein activating the catalyst complex causes a Dexter energy transfer from the activated transition metal complex to the reactive moiety to form a reactive intermediate.

11. The method of claim 1, wherein the biomolecule is within a 10 nm radius of the biomolecular antenna when the labeling agent binds the label moiety to the biomolecule.

12. A system for proximity-based labeling of intracellular molecules, comprising:
a cell;
a biomolecular antenna comprising:
a photocatalyst complex, comprising a photocatalyst and a ligand moiety, and
a binding agent complex, comprising a protein of interest coupled to a binding agent, wherein the binding agent is capable of binding the ligand moiety of the photocatalyst complex; and
a labeling agent comprising a label moiety and a reactive moiety, wherein the reactive moiety is configured to be activated to a reactive state by the photocatalyst complex;
wherein the biomolecular antenna and the labeling agent are each located within the cell.

13. The system of claim 12, wherein the binding agent comprises a haloalkane dehalogenase.

14. The system of claim 13, wherein the ligand moiety comprises an alkyl chloride.

15. The system of claim 14, wherein the alkyl chloride comprises 6 or more carbons.

16. The system of claim 12, wherein the binding agent is coupled to the protein of interest at the N-terminus or C-terminus of the protein of interest.

17. The system of claim 12, wherein the ligand moiety comprises 4,4′-di-tert-butyl-2,2′-dipyridyl, 2,2′-bipyridine, diphenhydramine-2,2′-bipyridine, 4-4′-dimethoxy-2-2′-bipyridine, dinapthalene-pyrene, phenanthroline, or diphenyl-phenanthroline.

18. The system of claim 12, wherein the photocatalyst comprises a transition metal.

19. The system of claim 18, wherein the transition metal has a triplet energy state greater than 60 kcal/mol.

20. The system of claim 18, wherein the transition metal is a platinum group metal.

21. The system of claim 20, wherein the transition metal is iridium or ruthenium.

22. The system of claim 21, wherein the transition metal is hexacoordinate.

23. The system of claim 18, wherein the transition metal absorbs light having wavelength from about 380 nm to about 700 nm.

24. The system of claim 18, wherein the transition metal has a visible light extinction coefficient greater than 1000 M⁻¹ cm⁻¹.

25. The system of claim 12, wherein the photocatalyst is an organocatalyst not including a transition metal.

26. The system of claim 25, wherein the organocatalyst comprises a thioxanthone group, phenothiazine group, flavin group, phenoxazine group, pthalazine group, quinoxaline group, quinazoline group, benzophenothiazine group, coumarin group, acetophenone group, or benzophenone group.

27. The system of claim 12, wherein the labeling agent is cell-permeable.

28. The system of claim 12, wherein the photocatalyst is capable of activating the labeling agent to form a reactive intermediate.

29. The system of claim 28, wherein the photocatalyst is capable of activating the labeling agent to form the reactive intermediate via Dexter energy transfer.

30. The system of claim 28, wherein the reactive intermediate has a diffusion radius of less than 10 nm prior to quenching.

31. The system of claim 28, wherein the reactive intermediate has a half-life (t_{1/2}) less than 2 ns.

32. The system of claim 12, wherein the binding agent is a protein, an E3 ligase, a polysaccharide, or a nucleic acid.

33. The system of claim 12, wherein the protein of interest is a K-Ras, a cMyc, a Src, a WRN, a Slug, a PARP1, an Aβ, a Tau, an influenza hemagglutinin, or a viral nucleoprotein.

34. A biomolecular assembly, comprising a dehalogenase coupled to a protein of interest and a photocatalyst.

35. The biomolecular assembly of claim 34, wherein the photocatalyst is configured to activate a labeling agent comprising a label moiety and a reactive moiety.

36. The biomolecular assembly of claim 35, wherein the photocatalyst is configured to activate the labeling agent after absorbing light having a wavelength from about 380 nm to about 700 nm.

37. The biomolecular assembly of claim 35, wherein the photocatalyst comprises a transition metal.

38. The biomolecular assembly of claim 37, wherein the transition metal is a platinum group metal.

39. The biomolecular assembly of claim 38, wherein the transition metal is iridium, tin, or ruthenium.

40. The biomolecular assembly of claim 37, wherein the transition metal is hexacoordinate.

41. The biomolecular assembly of claim 37, wherein the transition metal has a visible light extinction coefficient greater than 1000 M⁻¹ cm⁻¹.

42. The biomolecular assembly of claim 34, wherein the photocatalyst is an organocatalyst not including a transition metal.

43. The biomolecular assembly of claim 42, wherein the organocatalyst comprises a thioxanthone group, phenothiazine group, flavin group, phenoxazine group, pthalazine group, quinoxaline group, quinazoline group, benzophenothiazine group, coumarin group, acetophenone group, or benzophenone group.

44. The biomolecular assembly of claim 34, further comprising a photocatalyst complex, wherein the photocatalyst complex comprises the photocatalyst and a ligand moiety.

45. The biomolecular assembly of claim 44, wherein the ligand moiety comprises an alkyl chloride.

46. The biomolecular assembly of claim 45, wherein the alkyl chloride comprises 6 or more carbons.

47. A method of detecting a protein-protein interaction, comprising:
intracellularly expressing a first protein in a cell, the first protein coupled to a binding agent capable of binding to a catalyst complex, wherein the catalyst complex is a photocatalyst;
introducing the photocatalyst to the cell, thereby causing it to bind to the first protein;
introducing a labeling agent comprising a label moiety and a reactive moiety to the cell;
activating the photocatalyst, thereby activating the labeling agent by transfer of energy from the photocatalyst to the reactive moiety and causing the labeling agent to bind to a second protein within the cell; and
detecting the second protein by detecting the label moiety;
wherein the photocatalyst has the structure of Formula (I): 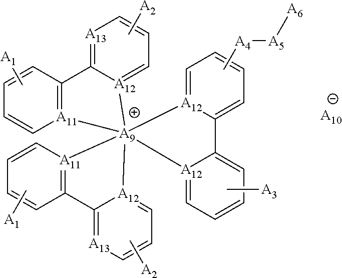
wherein:
A1 is present 0-4 times on the ring to which it is attached and each A1 is independently selected from CH₃, CF₃, F, Cl, N(CH₃)₂, and OR₁;
each R₁ is independently selected from H, a linear or branched alkyl group having 1-12 carbons, CHF₂, and CF₃;
A2 is present 0-4 times on the ring to which it is attached and each A2 is independently selected from CH₃, CF₃, F, Cl, N(CH₃)₂, and ORI;
A3 is present 0-4 times on the ring to which it is attached and each A3 is independently selected from CH₃, CF₃, F, Cl, N(CH₃)₂, and ORI;
A4 is selected from null, linear or branched alkyl group having 1-12 carbons, C₆₋₁₀ aryl, C₃₋₈ cycloalkyl, 4-10 membered heterocyclyl, and 5-10 membered heteroaryl, wherein said alkyl, aryl, cycloalkyl, heterocyclyl, and heteroaryl is optionally substituted with one or more C₁₋₆ alkyl, C₁₋₆ haloalkyl, halo, hydroxy, C₁₋₆ alkoxy, or amino;
A5 is selected from CONH NHCO, SONH, SO₂NH, NHSO, NHSO₂, NH, OCONH, NHCOO, C₁₋₆ alkoxy, and 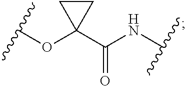 [SMILES: *NC(=O)C1(O*)CC1]
A6 is independently (PEG)a₁(CH₂)a₂C₁ or (PEG)a₁(CH₂)a₂(5-6 membered heterocyclyl)(PEG)a₁(CH₂)a₂Cl, wherein each a₁ is independently an integer from 0-10 and each a₂ is independently an integer from 6-10;
A9 is iridium;
A10 is an anion selected from tetraalkylborate, tetrafluoroborate, tetraphenylborate, chloride, cyanide, hexafluorophosphate (PF₆), bis(triphenylphosphine)iminium chloride, tetraphenylphosphonium chloride, and tetrabutylammonium;
each A11 and A12 are independently a C or N coordinated to A9; and
each A13 is independently CH or N.

48. The method of claim 47, wherein the photocatalyst has the structure:

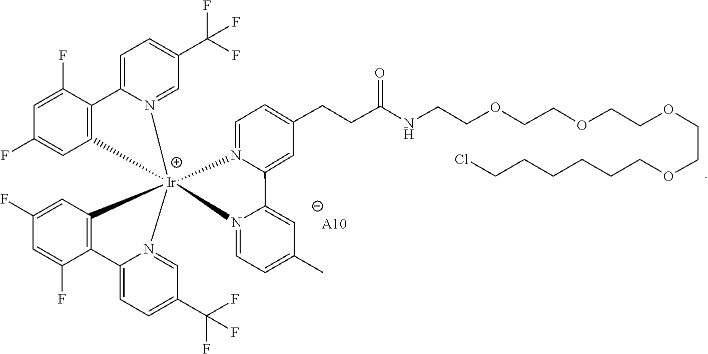

49. The method of claim 47, wherein the photocatalyst has the structure:

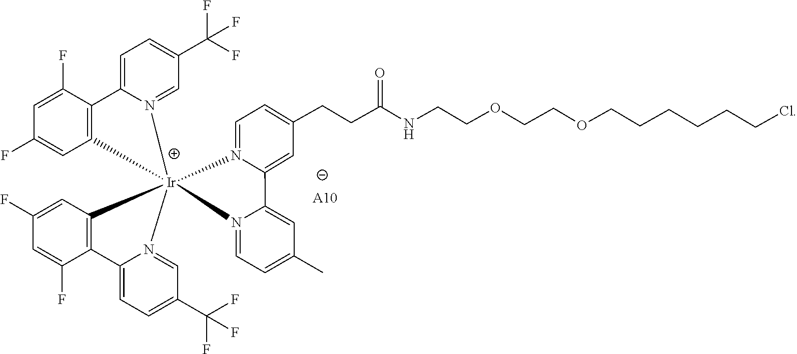

50. The method of claim 47, wherein the photocatalyst has the structure:

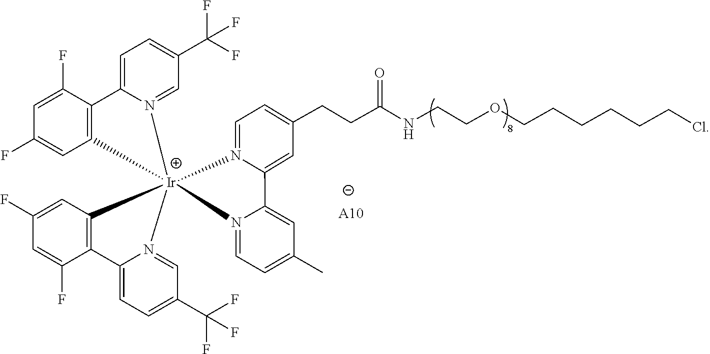

51. The method of claim 47, wherein the photocatalyst has the structure:

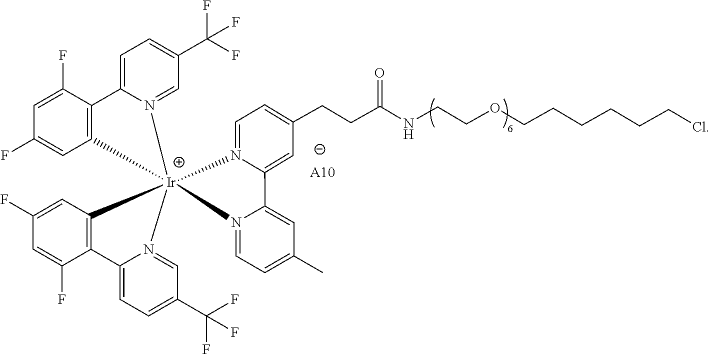

52. The method of claim 47, wherein the photocatalyst has the structure:

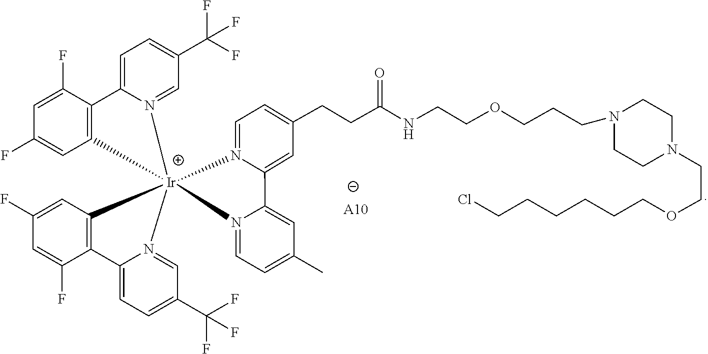

53. The method of claim 47, wherein the photocatalyst has the structure:

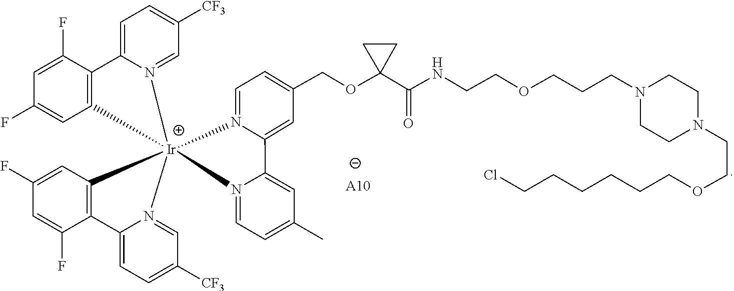

54. The method of claim 47, wherein the photocatalyst has the structure:

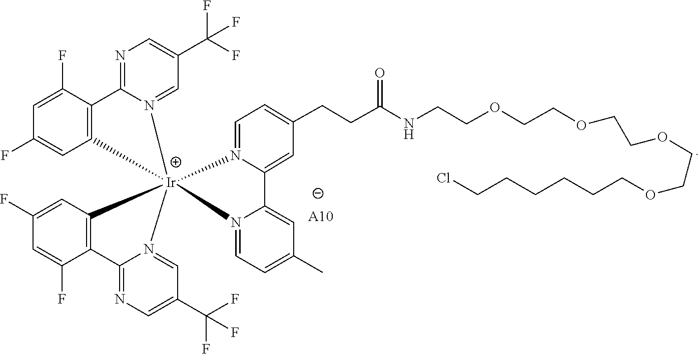

55. The method of claim 47, wherein the photocatalyst has the structure:

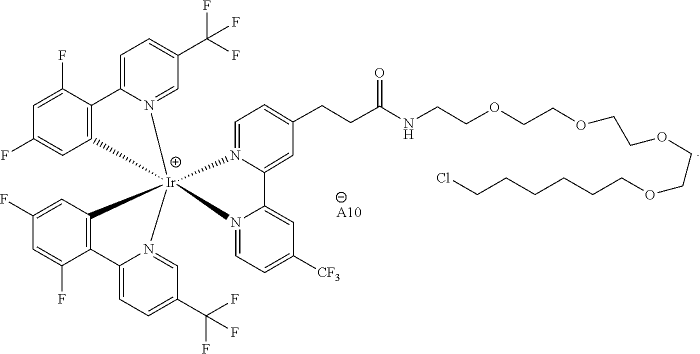

56. The method of claim 47, wherein the photocatalyst has the structure:

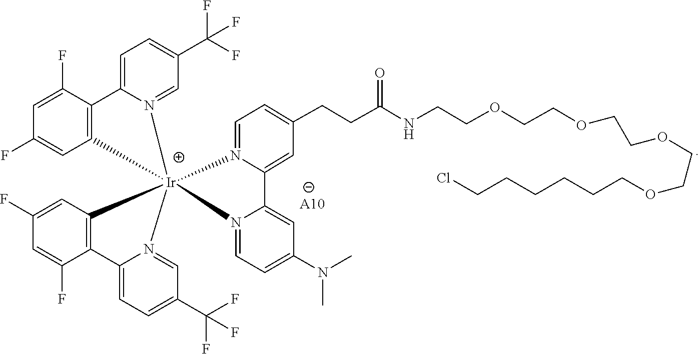

57. The method of claim 47, wherein the photocatalyst has the structure:

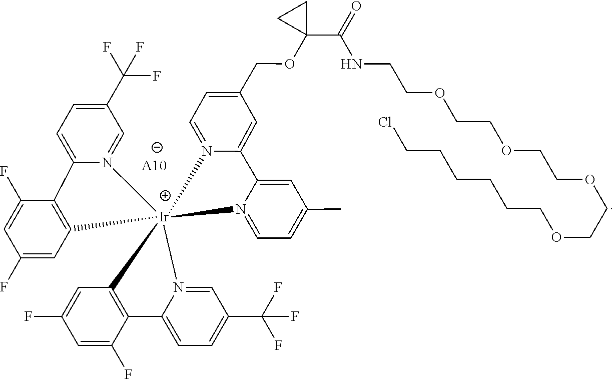

58. The method of claim 47, wherein A3 is present 0-4 times on the ring to which it is attached and each A3 is independently selected from CH₃, CF₃, F, Cl, and OR₁.

59. The method of claim 47, wherein A5 is selected from CONH, NHCO, SONH, SO₂NH, NHSO, NHSO₂, NH, OCONH, and NHCOO.

60. The method of claim 47, wherein A6 is (PEG)a₁(CH₂)a₂Cl, wherein a₁ is an integer from 0-10 and a₂ is an integer from 6-10.

61. The method of claim 47, wherein A13 is CH.

62. The method of claim 47, wherein the 5-6 membered heterocyclyl of A6 is piperazine or pyrrolidine.

63. A method of detecting a protein-protein interaction, comprising:
intracellularly expressing a first protein in a cell, the first protein coupled to a binding agent capable of binding to a catalyst complex;
introducing the catalyst complex to the cell, thereby causing it to bind to the first protein;
introducing a labeling agent comprising a label moiety and a reactive moiety to the cell;
activating the catalyst complex, thereby activating the labeling agent by transfer of energy from the catalyst complex to the reactive moiety and causing the labeling agent to bind to a second protein within the cell; and
detecting the second protein by detecting the label moiety;
wherein the labeling agent has the structure of Formula (III-a): 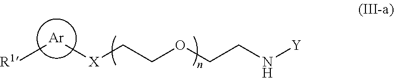
wherein:
R¹′ is selected from an azide, a methyl diazirine, a trifluoromethyl diazirine, and a phenyl diazirine;
Ring Ar is selected from phenyl, pyridyl, pyrimidyl, pyrazinyl, pyridizynyl, naphthyl, and quinolinyl, optionally substituted with one or more —OH, —OMe, —OEt, —OCF₃, —OCF₂H, —NHMe, —NMe₂, —F, —Cl, —Br, -Me, or -Et;
X is selected from O, NH, NR², CH₂NHCO, CONH, CONR², SO₂NH, and SO₂NR²;
R² is selected from H, OMe, Me, and Et;
n is 0, 1, 2, 3, 4, 5, or 6; and
Y is a biotin-linked amide or an amide-linked fluorescent dye.

64. The method of claim 63, wherein the labeling agent has the structure:

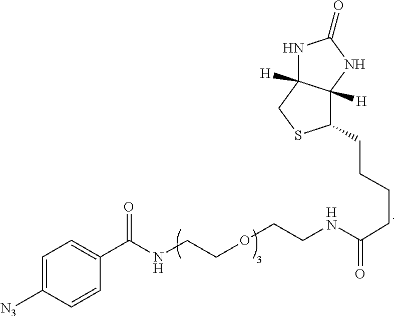

65. The method of any one of claims 47-64, wherein the first protein is a ubiquitin ligase.

66. A method of detecting a protein-protein interaction, comprising:
intracellularly expressing a first protein in a cell, the first protein coupled to a binding agent capable of binding to a catalyst complex, wherein the first protein is a ubiquitin ligase;
introducing the catalyst complex to the cell, thereby causing it to bind to the first protein;
introducing a labeling agent comprising a label moiety and a reactive moiety to the cell;
activating the catalyst complex, thereby activating the labeling agent by transfer of energy from the catalyst complex to the reactive moiety and causing the labeling agent to bind to a second protein within the cell; and
detecting the second protein by detecting the label moiety.

67. The method of any one of claims 63-66, wherein the catalyst complex is a photocatalyst.

68. The method of claim 67, wherein the photocatalyst has the structure of Formula (I): 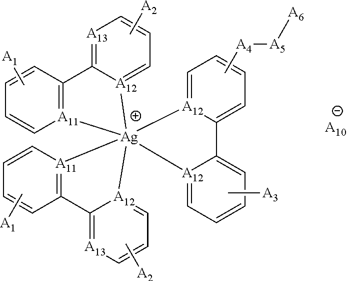
wherein:
A1 is present 0-4 times on the ring to which it is attached and each A1 is independently selected from CH₃, CF₃, F, Cl, N(CH₃)₂, and OR₁;
each R₁ is independently selected from H, a linear or branched alkyl group having 1-12 carbons, CHF₂, and CF₃;
A2 is present 0-4 times on the ring to which it is attached and each A2 is independently selected from CH₃, CF₃, F, Cl, N(CH₃)₂, and OR₁;
A3 is present 0-4 times on the ring to which it is attached and each A3 is independently selected from CH₃, CF₃, F, Cl, N(CH₃)₂, and OR₁;
A4 is selected from null, linear or branched alkyl group having 1-12 carbons, C₆₋₁₀ aryl, C₃₋₈ cycloalkyl, 4-10 membered heterocyclyl, and 5-10 membered heteroaryl, wherein said alkyl, aryl, cycloalkyl, heterocyclyl, and heteroaryl is optionally substituted with one or more C₁₋₆ alkyl, C₁₋₆ haloalkyl, halo, hydroxy, C₁_6 alkoxy, or amino;
A5 is selected from CONH, NHCO, SONH, SO₂NH, NHSO, NHSO₂, NH, OCONH, NHCOO, C₁₋₆ alkoxy, and 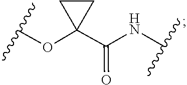 [SMILES: *NC(=O)C1(O*)CC1]
A6 is independently (PEG)a₁(CH₂)a₂Cl or (PEG)a₁(CH₂)a₂(5-6 membered heterocyclyl)(PEG)a₁(CH₂)a₂Cl, wherein each a₁ is independently an integer from 0-10 and each a₂ is independently an integer from 6-10;
A9 is a D9 metal selected from copper, vanadium, chromium, scandium, titanium, manganese, iron, cobalt, nickel, copper, zinc, yttrium, zirconium, niobium, molybdenum, technetium, ruthenium, rhodium, palladium, silver, cadmium lanthanum, hafnium, tantalum, tungsten, rhenium, osmium, iridium, platinum, gold, and mercury;
A10 is an anion selected from tetraalkylborate, tetrafluoroborate, tetraphenylborate, chloride, cyanide, hexafluorophosphate (PF₆), bis(triphenylphosphine)iminium chloride, tetraphenylphosphonium chloride, and tetrabutylammonium;
A11 and A12 are independently a C or N coordinated to A9; and
each A13 is independently a CH or N.

69. The method of claim 68, wherein the photocatalyst has the structure:

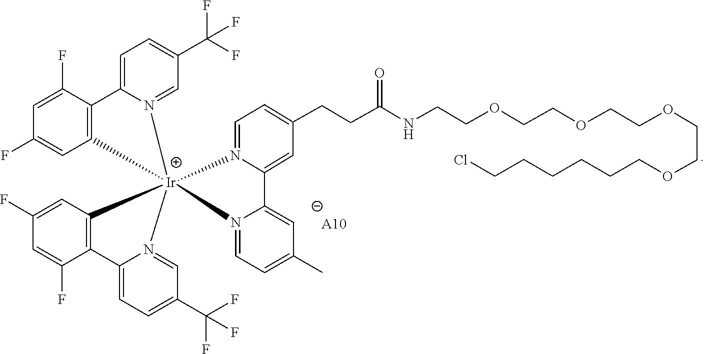

70. The method of claim 68, wherein the photocatalyst has the structure:

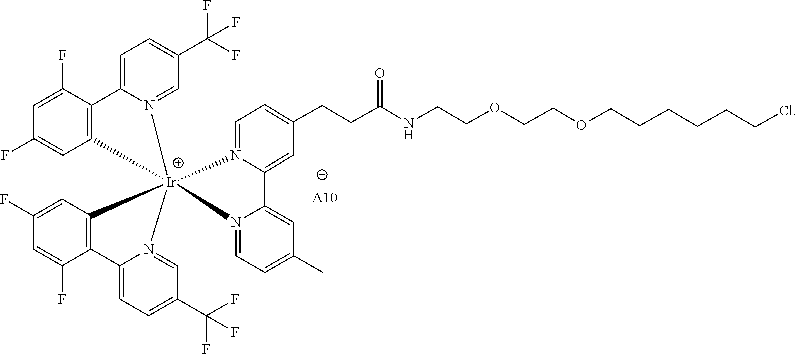

71. The method of claim 68, wherein the photocatalyst has the structure:

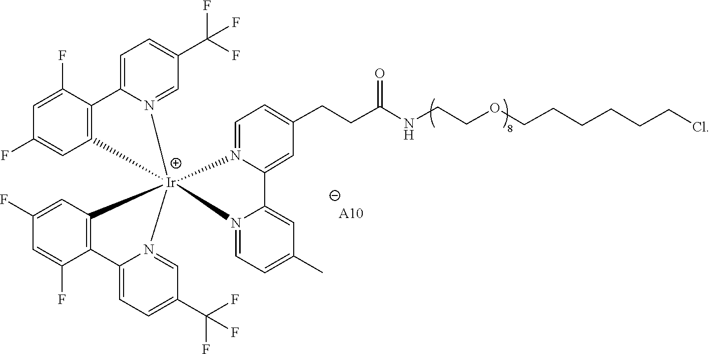

72. The method of claim 68, wherein the photocatalyst has the structure:

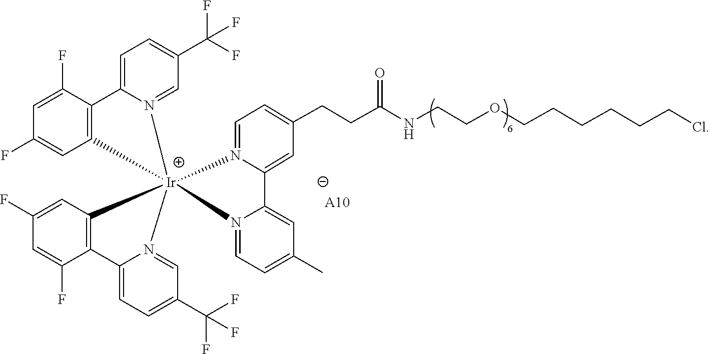

73. The method of claim 68, wherein the photocatalyst has the structure:

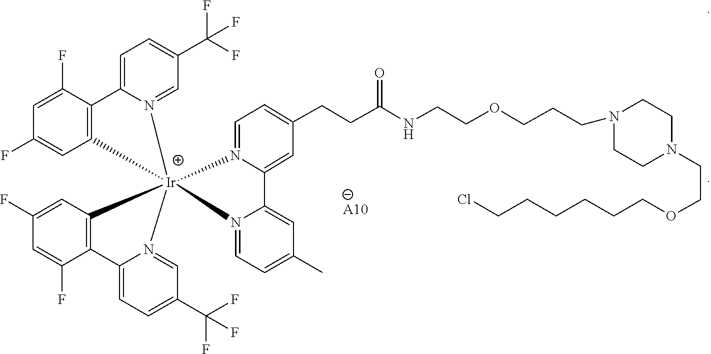

74. The method of claim 68, wherein the photocatalyst has the structure:

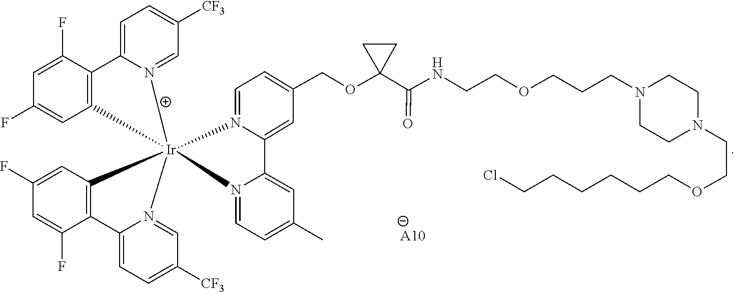

75. The method of claim 68, wherein the photocatalyst has the structure:

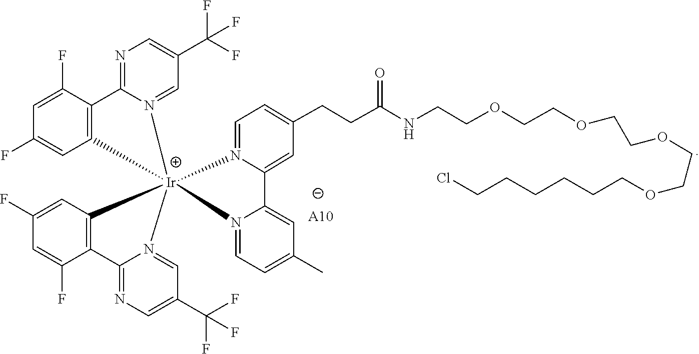

76. The method of claim 68, wherein the photocatalyst has the structure:

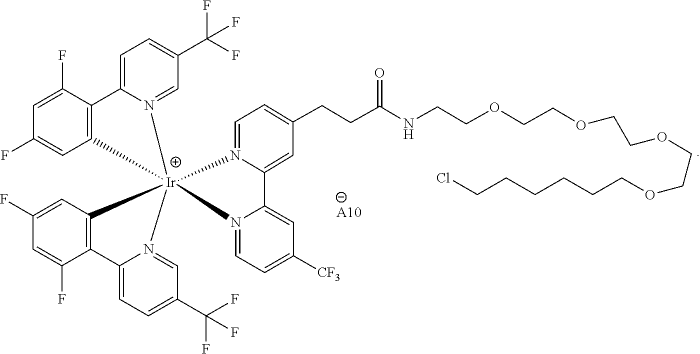

77. The method of claim 68, wherein the photocatalyst has the structure:

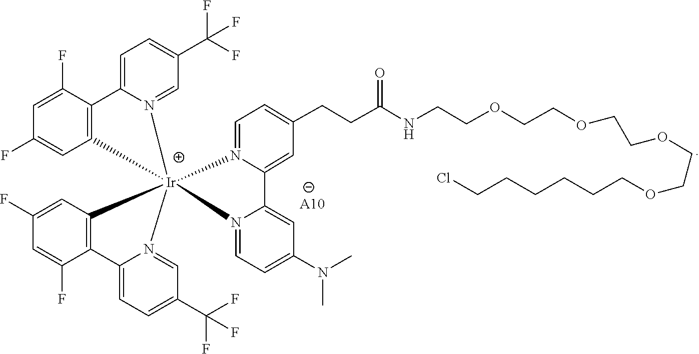

78. The method of claim 68, wherein the photocatalyst has the structure:

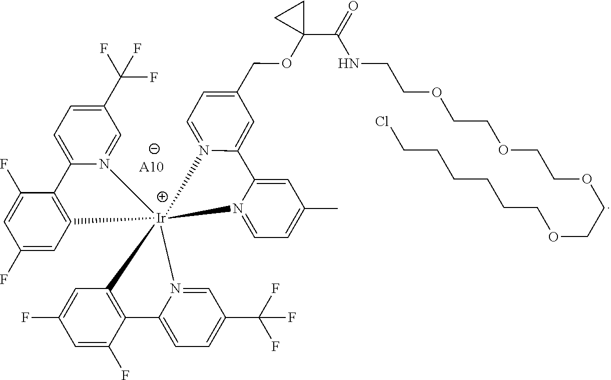

79. The method of claim 68, wherein A3 is present 0-4 times on the ring to which it is attached and each A3 is independently selected from CH₃, CF₃, F, Cl, and OR₁.

80. The method of claim 68, wherein A5 is selected from CONH, NHCO, SONH, SO₂NH, NHSO, NHSO₂, NH, OCONH, and NHCOO.

81. The method of claim 68, wherein A6 is (PEG)a₁(CH₂)a₂Cl, wherein a₁ is an integer from 0-10 and a₂ is an integer from 6-10.

82. The method of claim 68, wherein A13 is CH.

83. The method of claim 68, wherein the 5-6 membered heterocyclyl of A6 is piperazine or pyrrolidine.

84. The method of any one of claims 47, 63, and 66, wherein the labeling agent has the structure of Formula (III-a): 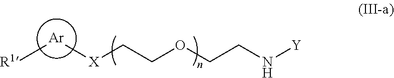
wherein:
R¹′ is selected from an azide, a methyl diazirine, a trifluoromethyl diazirine, and a phenyl diazirine;
Ring Ar is selected from phenyl, pyridyl, pyrimidyl, pyrazinyl, pyridizynyl, naphthyl, and quinolinyl, optionally substituted with one or more —OH, —OMe, —OEt, —OCF₃, —OCF₂H, —NHMe, —NMe₂, —F, —Cl, —Br, -Me, or -Et;
X is selected from 0, NH, NR², CH₂NHCO, CONH, CONR², SO₂NH, and SO₂NR²;
R² is selected from H, OMe, Me, and Et;
n is 0, 1, 2, 3, 4, 5, or 6; and
Y is a biotin-linked amide or an amide-linked fluorescent dye.

85. The method of claim 84, wherein the labeling agent has the structure:

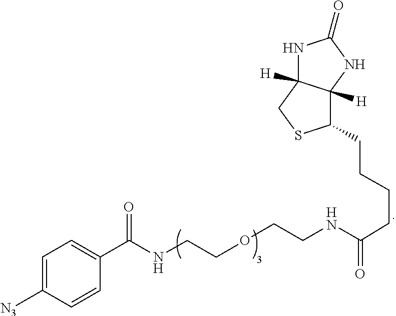

86. The method of any one of claims 47-85, wherein said method is conducted in the absence of an exogenous compound that promotes interaction between the first and second proteins.

87. The method of any one of claims 47-85, wherein said method is conducted in the presence of a test compound, wherein detecting the second protein or a level of the second protein indicates that the test compound promotes interaction between the first and second proteins.

88. The method of any one of claims 47-87, wherein during the activating of the catalyst complex, the cell is a live cell.

89. The method of any one of claims 47-88, wherein the binding agent comprises a haloalkane dehalogenase.

90. The method of any one of claims 47-89, wherein activating the catalyst complex comprises shining light on the cell, wherein the light has a wavelength from about 380 nm to about 700 nm.

91. The method of any one of claims 47-90, wherein activating the catalyst complex causes a Dexter energy transfer from the activated catalyst complex to the reactive moiety to form a reactive intermediate.

92. A cell, comprising:
a first protein coupled to a haloalkane dehalogenase;
a photocatalyst having the structure of Formula (T): 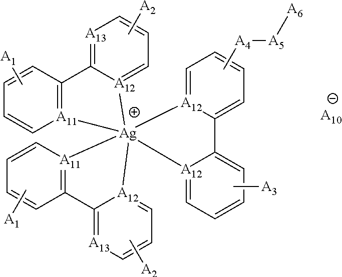
wherein:
A1 is present 0-4 times on the ring to which it is attached and each A1 is independently selected from CH₃, CF₃, F, Cl, N(CH₃)₂, and OR₁;
each R₁ is independently selected from H, a linear or branched alkyl group having 1-12 carbons, CHF₂, and CF₃;
A2 is present 0-4 times on the ring to which it is attached and each A2 is independently selected from CH₃, CF₃, F, Cl, N(CH₃)₂, and OR₁;
A3 is present 0-4 times on the ring to which it is attached and each A3 is independently selected from CH₃, CF₃, F, Cl, N(CH₃)₂, and OR₁;
A4 is selected from null, linear or branched alkyl group having 1-12 carbons, C₆₋₁₀ aryl, C₃₋₈ cycloalkyl, 4-10 membered heterocyclyl, and 5-10 membered heteroaryl, wherein said alkyl, aryl, cycloalkyl, heterocyclyl, and heteroaryl is optionally substituted with one or more C₁₋₆ alkyl, C₁₋₆ haloalkyl, halo, hydroxy, C₁₋₆ alkoxy, or amino;
A5 is selected from CONH, NHCO, SONH, SO₂NH, NHSO, NHSO₂, NH, OCONH, NHCOO, C₁₋₆ alkoxy, and 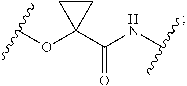 [SMILES: *NC(=O)C1(O*)CC1]
A6 is independently (PEG)a₁(CH₂)a₂Cl or (PEG)a₁(CH₂)a₂(5-6 membered heterocyclyl)(PEG)a₁(CH₂)a₂Cl, wherein each a₁ is independently an integer from 0-10 and each a₂ is independently an integer from 6-10;
A9 is a D9 metal selected from copper, vanadium, chromium, scandium, titanium, manganese, iron, cobalt, nickel, copper, zinc, yttrium, zirconium, niobium, molybdenum, technetium, ruthenium, rhodium, palladium, silver, cadmium lanthanum, hafnium, tantalum, tungsten, rhenium, osmium, iridium, platinum, gold, and mercury; and
A11 and A12 are independently a C or N coordinated to A9;
each A13 is independently CH or N; and
a labeling agent having the structure of Formula (III-a): 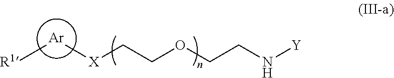
wherein:
R¹′ is selected from an azide, a methyl diazirine, a trifluoromethyl diazirine, and a phenyl diazirine;
Ring Ar is selected from phenyl, pyridyl, pyrimidyl, pyrazinyl, pyridizynyl, naphthyl, and quinolinyl, optionally substituted with one or more —OH, —OMe, —OEt, —OCF₃, —OCF₂H, —NHMe, —NMe₂, —F, —Cl, —Br, -Me, or -Et;
X is selected from O, NH, NR², CH₂NHCO, CONH, CONR², SO₂NH, and SO₂NR²;
R² is selected from H, OMe, Me, and Et;
n is 0, 1, 2, 3, 4, 5, or 6; and
Y is a biotin-linked amide or an amide-linked fluorescent dye.

93. The cell of claim 92, wherein the photocatalyst has the structure:

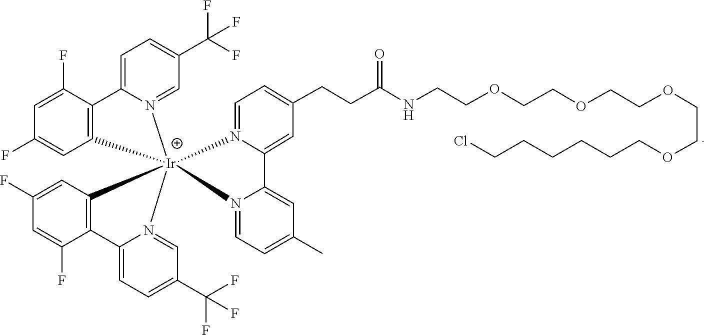

94. The cell of claim 92, wherein the photocatalyst has the structure:

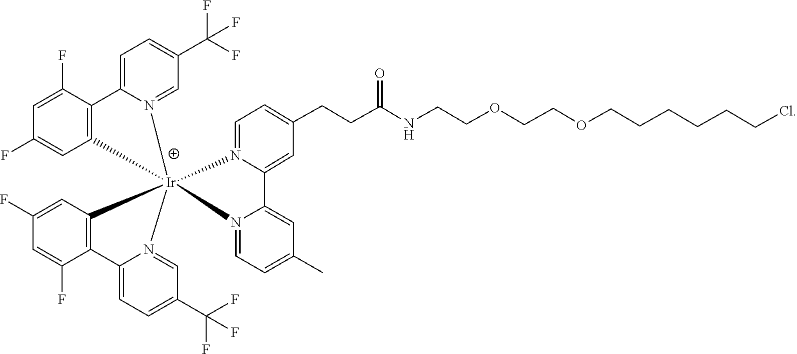

95. The cell of claim 92, wherein the photocatalyst has the structure:

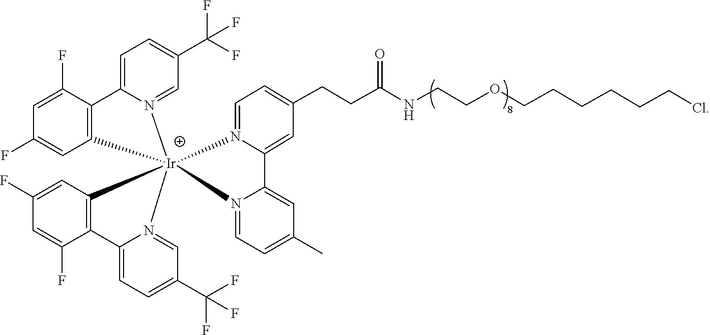

96. The cell of claim 92, wherein the photocatalyst has the structure:

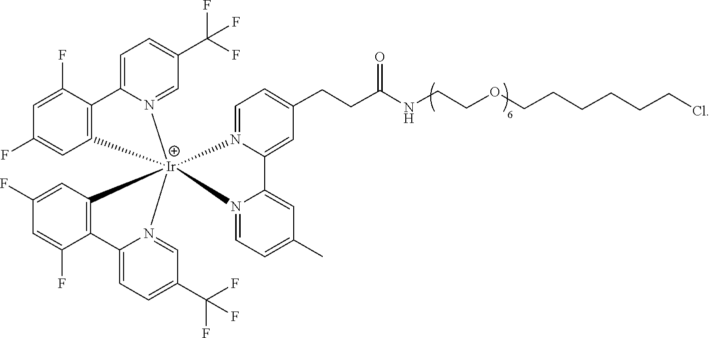

97. The cell of claim 92, wherein the photocatalyst has the structure:

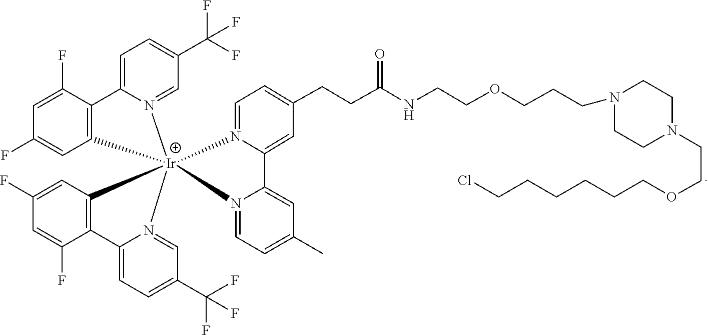

98. The cell of claim 92, wherein the photocatalyst as the structure:

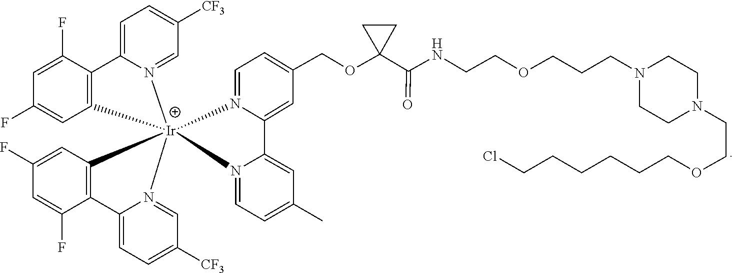

99. The cell of claim 92, wherein the photocatalyst has the structure:

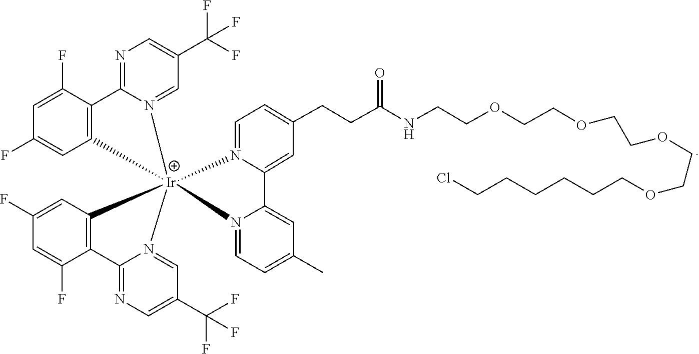

100. The cell of claim 92, wherein the photocatalyst has the structure:

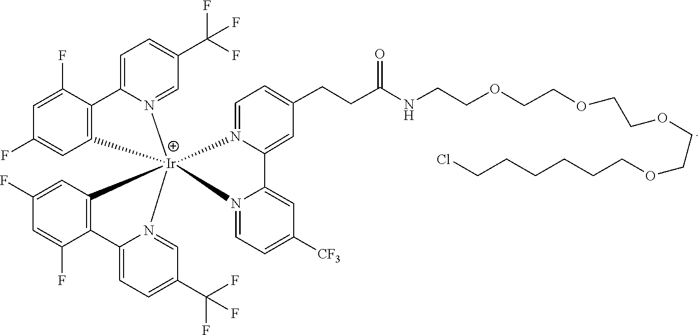

101. The cell of claim 92, wherein the photocatalyst has the structure:

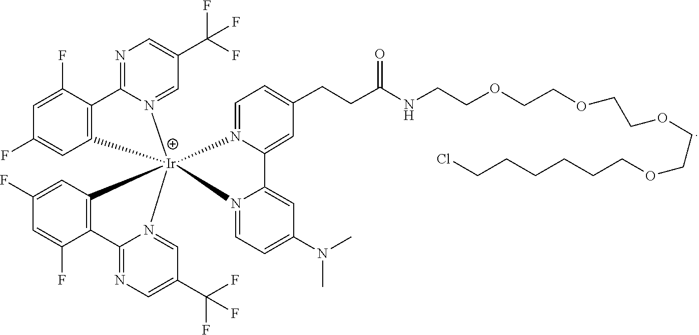

102. The cell of claim 92, wherein the photocatalyst has the structure:

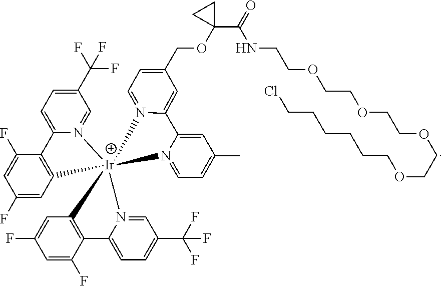

103. The cell of claim 92, wherein A3 is present 0-4 times on the ring to which it is attached and each A3 is independently selected from CH₃, CF₃, F, Cl, and OR₁.

104. The cell of claim 92, wherein A5 is selected from CONH, NHCO, SONH, SO₂NH, NHSO, NHSO₂, NH, OCONH, and NHCOO.

105. The cell of claim 92, wherein A6 is (PEG)a₁(CH₂)a₂C₁, wherein a₁ is an integer from 0-10 and a₂ is an integer from 6-10.

106. The cell of claim 92, wherein A13 is CH.

107. The cell of claim 92, wherein the 5-6 membered heterocyclyl of A6 is piperazine or pyrrolidine.

108. The cell of claim any one of claims 92-107, wherein the labeling agent has the structure:

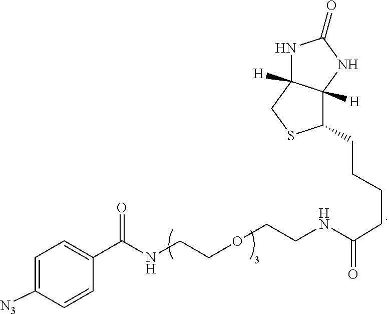

109. A protein complex, comprising the structure:
P1-P2-Cat
wherein:
P1 is a ubiquitin ligase;
P2 is a haloalkane dehalogenase;
Cat is a photocatalyst having the structure of Formula (I): 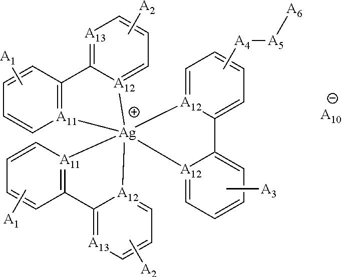
wherein:
A1 is present 0-4 times on the ring to which it is attached and each A1 is independently selected from CH₃, CF₃, F, Cl, N(CH₃)₂, and OR₁;
each R₁ is independently selected from H, a linear or branched alkyl group having 1-12 carbons, CHF₂, and CF₃;
A2 is present 0-4 times on the ring to which it is attached and each A2 is independently selected from CH₃, CF₃, F, Cl, N(CH₃)₂, and OR₁;
A3 is present 0-4 times on the ring to which it is attached and each A3 is independently selected from CH₃, CF₃, F, Cl, N(CH₃)₂, and OR₁;
A4 is selected from null, linear or branched alkyl group having 1-12 carbons, C₆₋₁₀ aryl, C₃₋₈ cycloalkyl, 4-10 membered heterocyclyl, and 5-10 membered heteroaryl, wherein said alkyl, aryl, cycloalkyl, heterocyclyl, and heteroaryl is optionally substituted with one or more C₁₋₆ alkyl, C₁₋₆ haloalkyl, halo, hydroxy, C₁₋₆ alkoxy, or amino;
A5 is selected from CONH, NHCO, SONH, SO₂NH, NHSO, NHSO₂, NH, OCONH, NHCOO, C₁₋₆ alkoxy, and 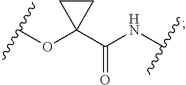 [SMILES: *NC(=O)C1(O*)CC1]
A6 is independently (PEG)a₁(CH₂)a₂Cl or (PEG)a₁(CH₂)a₂(5-6 membered heterocyclyl)(PEG)a₁(CH₂)a₂Cl, wherein each a₁ is independently an integer from 0-10 and each a₂ is independently an integer from 6-10;
A9 is a D9 metal selected from copper, vanadium, chromium, scandium, titanium, manganese, iron, cobalt, nickel, copper, zinc, yttrium, zirconium, niobium, molybdenum, technetium, ruthenium, rhodium, palladium, silver, cadmium lanthanum, hafnium, tantalum, tungsten, rhenium, osmium, iridium, platinum, gold, and mercury;
A11 and A12 are independently a C or N coordinated to A9; and
each A13 is independently CH or N.

110. The protein complex of claim 109, wherein Cat has the structure:

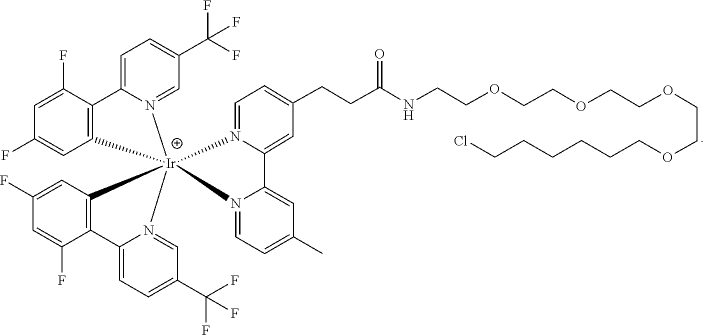

111. The protein complex of claim 109, wherein Cat has the structure:

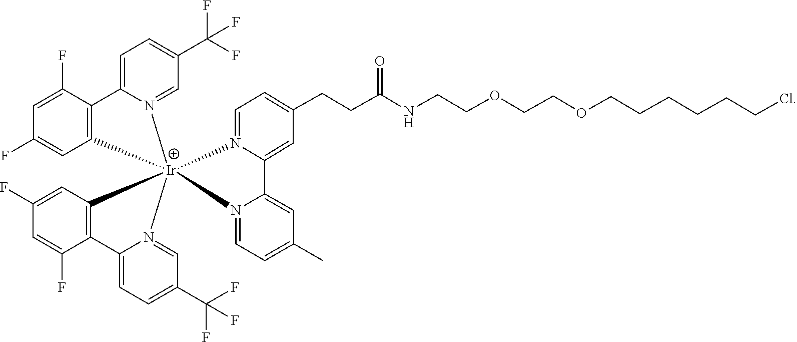

112. The protein complex of claim 109, wherein Cat has the structure:

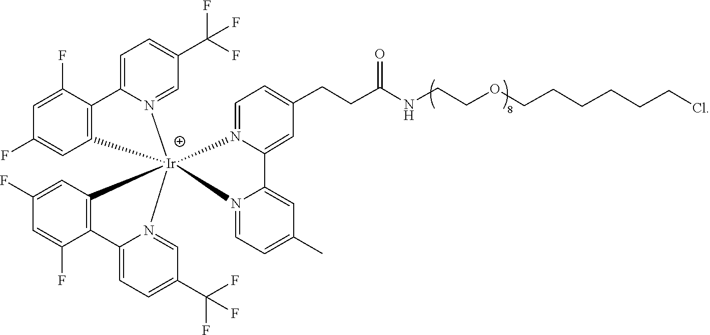

113. The protein complex of claim 109, wherein Cat has the structure:

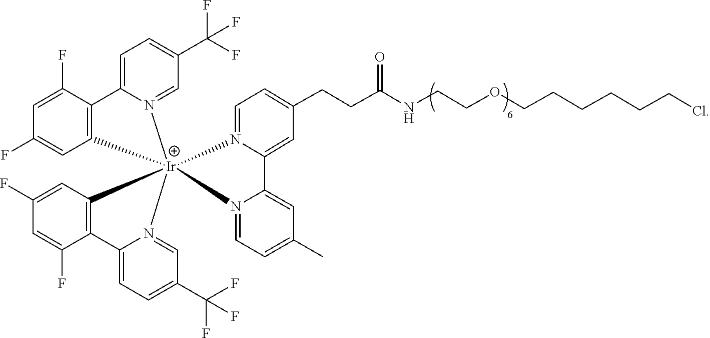

114. The protein complex of claim 109, wherein Cat has the structure:

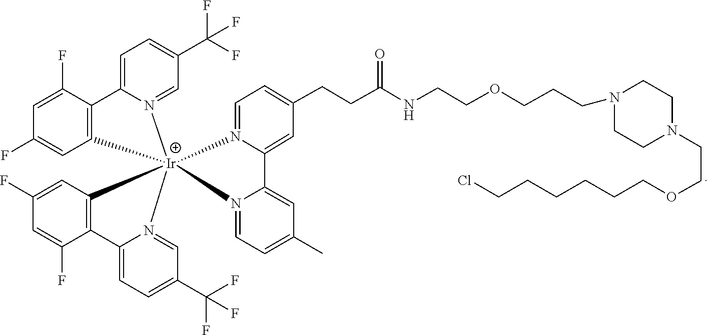

115. The protein complex of claim 109, wherein Cat has the structure:

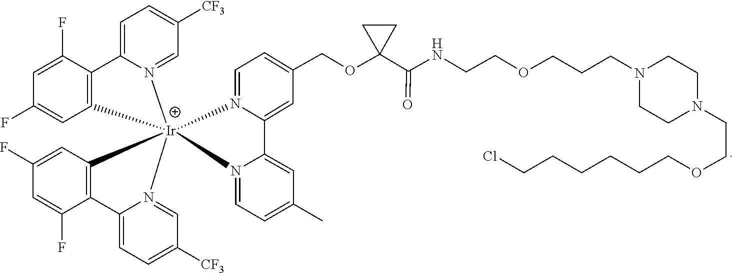

116. The protein complex of claim 109, wherein Cat has the structure:

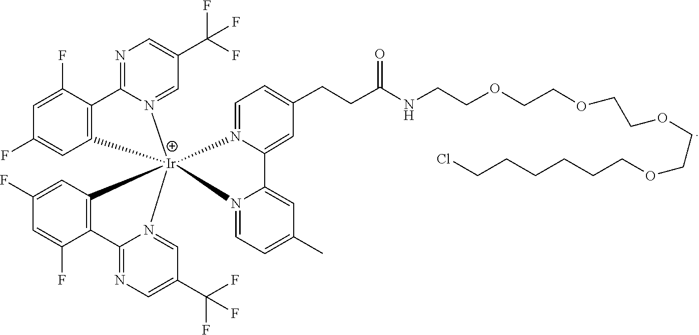

117. The protein complex of claim 109, wherein Cat has the structure:

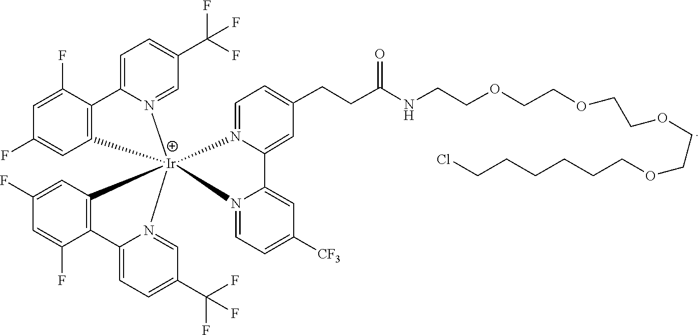

118. The protein complex of claim 109, wherein Cat has the structure:

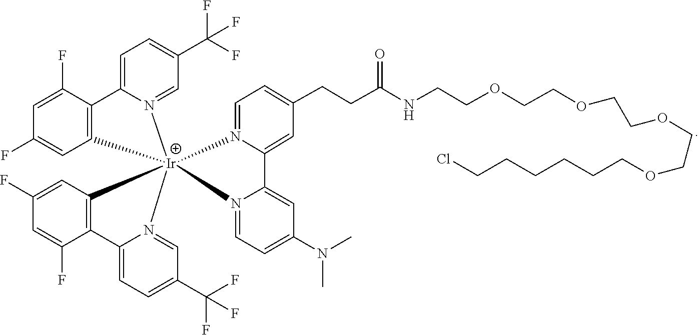

119. The protein complex of claim 109, wherein Cat has the structure:

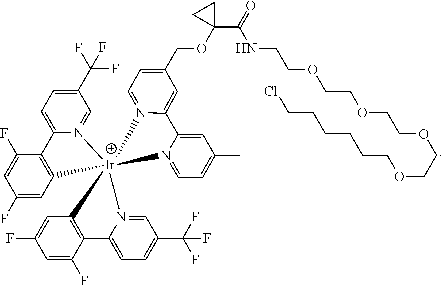

120. The protein complex of claim 109, wherein is present 0-4 times on the ring to which it is attached and each A3 is independently selected from CH₃, CF₃, F, Cl, N(CH₃)₂, and OR₁.

121. The protein complex of claim 109, wherein A5 is selected from CONH, NHCO, SONH, SO₂NH, NHSO, NHSO₂, NH, OCONH, and NHCOO.

122. The protein complex of claim 109, wherein A6 is (PEG)a₁(CH₂)a₂C₁, wherein a₁ is an integer from 0-10 and a₂ is an integer from 6-10.

123. The protein complex of claim 109, wherein A13 is CH.

124. The protein complex of claim 109, wherein the 5-6 membered heterocyclyl of A6 is piperazine or pyrrolidine.

125. A cell, comprising:
the protein complex of any one of claims 109-119; and
a labeling agent having the structure of Formula (III-a): 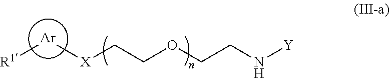
wherein:
R¹′ is selected from an azide, a methyl diazirine, a trifluoromethyl diazirine, and a phenyl diazirine;
Ring Ar is selected from phenyl, pyridyl, pyrimidyl, pyrazinyl, pyridizynyl, naphthyl, and quinolinyl, optionally substituted with one or more —OH, —OMe, —OEt, —OCF₃, —OCF₂H, —NHMe, —NMe₂, —F, —Cl, —Br, -Me, or -Et;
X is selected from O, NH, NR², CH₂NHCO, CONH, CONR², SO₂NH, and SO₂NR²;
R² is selected from H, OMe, Me, and Et;
n is 0, 1, 2, 3, 4, 5, or 6; and
Y is a biotin-linked amide or an amide-linked fluorescent dye.

126. A cell, comprising a nucleotide sequence expressing a fusion protein comprising a ubiquitin ligase and a haloalkane dehalogenase.

127. A photocatalyst having the structure of Formula (I): 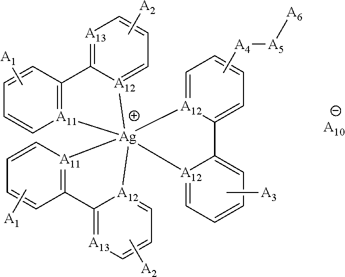
wherein:
A1 is present 0-4 times on the ring to which it is attached and each A1 is independently selected from CH₃, CF₃, F, Cl, N(CH₃)₂, and OR₁;
each R₁ is independently selected from H, a linear or branched alkyl group having 1-12 carbons, CHF₂, and CF₃;
A2 is present 0-4 times on the ring to which it is attached and each A2 is independently selected from CH₃, CF₃, F, Cl, N(CH₃)₂, and OR₁;
A3 is present 0-4 times on the ring to which it is attached and each A3 is independently selected from CH₃, CF₃, F, Cl, N(CH₃)₂, and OR₁;
A4 is selected from null, linear or branched alkyl group having 1-12 carbons, C₆₋₁₀ aryl, C₃₋₈ cycloalkyl, 4-10 membered heterocyclyl, and 5-10 membered heteroaryl, wherein said alkyl, aryl, cycloalkyl, heterocyclyl, and heteroaryl is optionally substituted with one or more C₁₋₆ alkyl, C₁₋₆ haloalkyl, halo, hydroxy, C₁₋₆ alkoxy, or amino;
A5 is selected from CONH, NHCO, SONH, SO₂NH, NHSO, NHSO₂, NH, OCONH, NHCOO, C₁₋₆ alkoxy, and 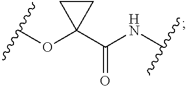 [SMILES: *NC(=O)C1(O*)CC1]
A6 is independently (PEG)a₁(CH₂)a₂C₁ or (PEG)a₁(CH₂)a₂(5-6 membered heterocyclyl)(PEG)a₁(CH₂)a₂Cl, wherein each a₁ is independently an integer from 0-10 and each a₂ is independently an integer from 6-10;
A9 is a D9 metal selected from copper, vanadium, chromium, scandium, titanium, manganese, iron, cobalt, nickel, copper, zinc, yttrium, zirconium, niobium, molybdenum, technetium, ruthenium, rhodium, palladium, silver, cadmium lanthanum, hafnium, tantalum, tungsten, rhenium, osmium, iridium, platinum, gold, and mercury;
A11 and A12 are independently a C or N coordinated to A9; and
each A13 is independently CH or N,
wherein the photocatalyst is not

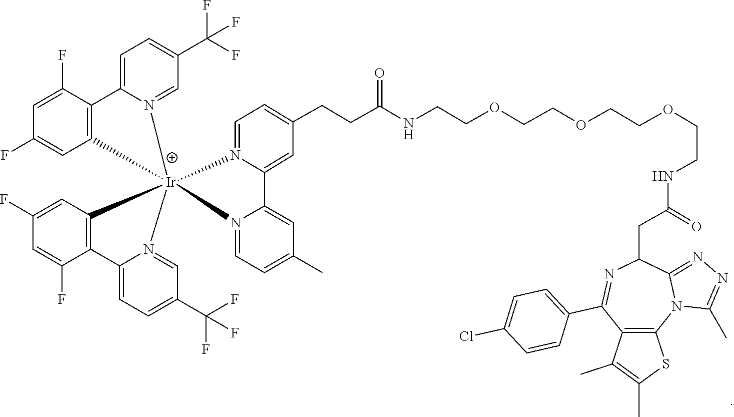

128. The photocatalyst of claim 127, wherein A3 is present 0-4 times on the ring to which it is attached and each A3 is independently selected from CH₃, CF₃, F, Cl, N(CH₃)₂ and OR₁.

129. The photocatalyst of claim 127, wherein A5 is selected from CONH, NHCO, SONH, SO₂NH, NHSO, NHSO₂, NH, OCONH, and NHCOO.

130. The photocatalyst of claim 127, wherein A6 is (PEG)a₁(CH₂)a₂Cl, wherein a₁ is an integer from 0-10 and a₂ is an integer from 6-10.

131. The photocatalyst of claim 127, wherein A13 is CH.

132. The photocatalyst of claim 127, wherein the 5-6 membered heterocyclyl of A6 is piperazine or pyrrolidine.

133. The photocatalyst of claim 127, wherein the photocatalyst has the structure:

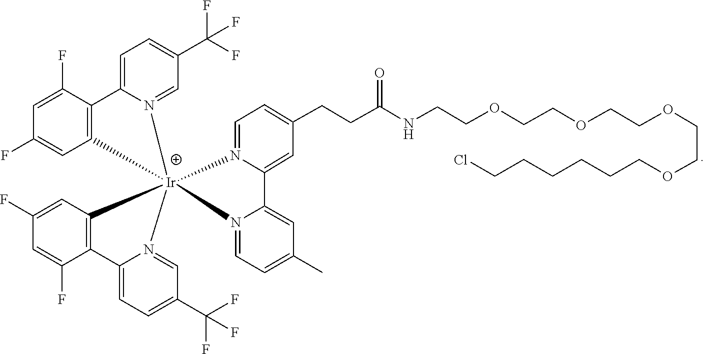

134. The photocatalyst of claim 127, wherein the photocatalyst has the structure:

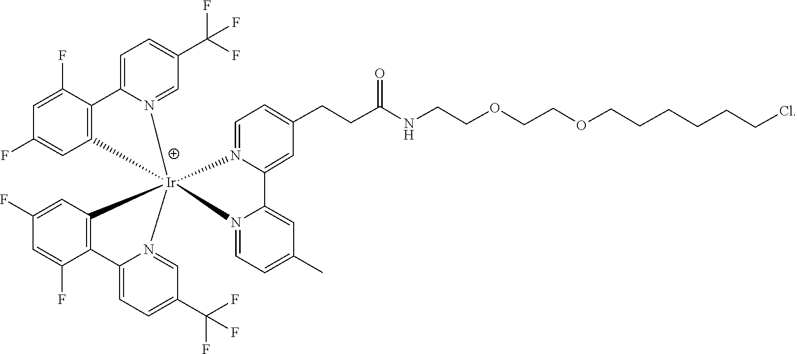

135. The photocatalyst of claim 127, wherein the photocatalyst has the structure:

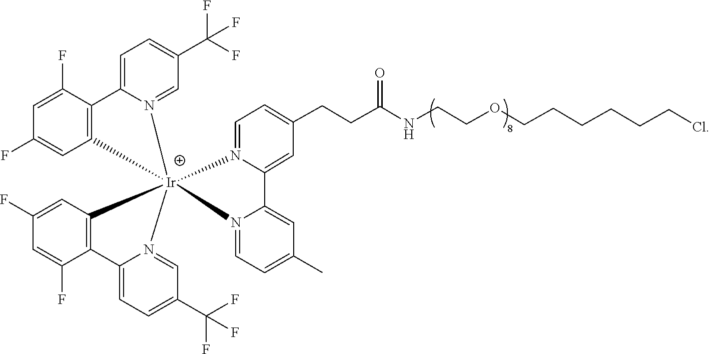

136. The photocatalyst of claim 127, wherein the photocatalyst has the structure:

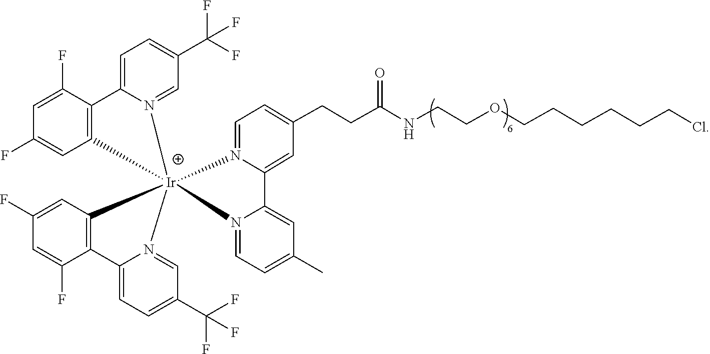

137. The photocatalyst of claim 127, wherein the photocatalyst has the structure:

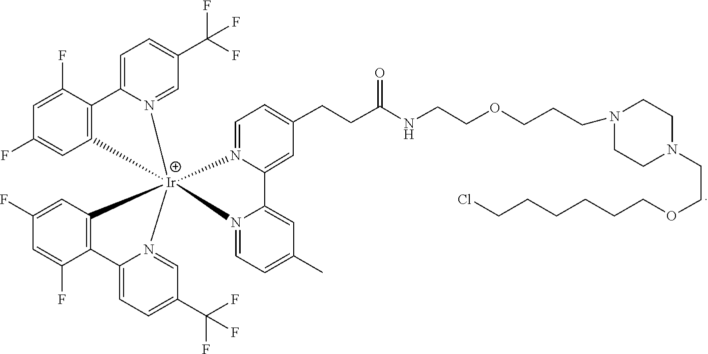

138. The photocatalyst of claim 127, wherein the photocatalyst has the structure:

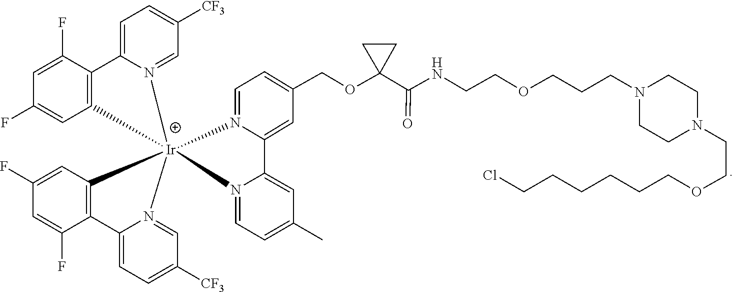

139. The photocatalyst of claim 127, wherein the photocatalyst has the structure:

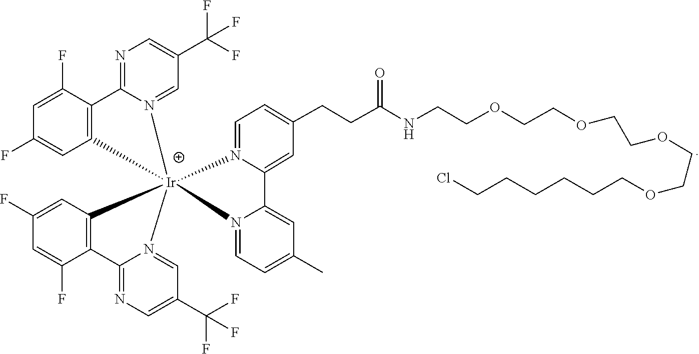

140. The photocatalyst of claim 127, wherein the photocatalyst has the structure:

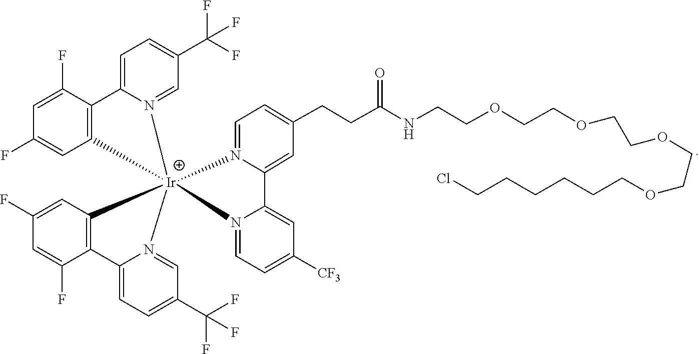

141. The photocatalyst of claim 127, wherein the photocatalyst has the structure:

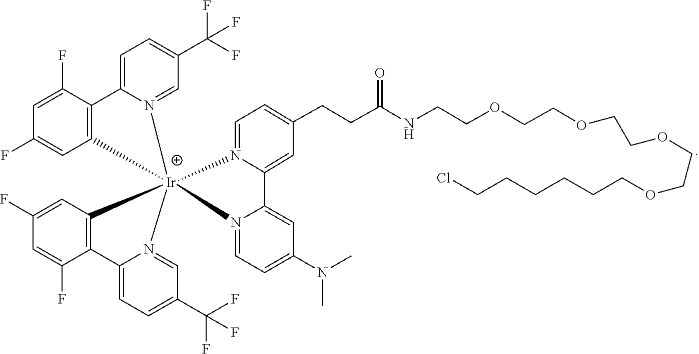

142. The photocatalyst of claim 127, wherein the photocatalyst has the structure: [chemical structure]
